SEQUENCE LISTING (121 sequences)

SEQ ID NO: 1 - DNA, 20 bp, Artificial Sequence
  Primer
tgctggatcacaaatcctca

SEQ ID NO: 2 - DNA, 20 bp, Artificial Sequence
  Primer
atagcgcctggactcctttt

SEQ ID NO: 3 - DNA, 20 bp, Artificial Sequence
  Primer
cagttcagcgagttcaagca

SEQ ID NO: 4 - DNA, 20 bp, Artificial Sequence
  Primer
tcacgcagtagagcaccatc

SEQ ID NO: 5 - DNA, 20 bp, Artificial Sequence
  Primer
gccaaggagatatgggacat

SEQ ID NO: 6 - DNA, 20 bp, Artificial Sequence
  Primer
agcaccgtgggtcattattc

SEQ ID NO: 7 - DNA, 20 bp, Artificial Sequence
  Primer
ttgtctggtccatcctcctc

SEQ ID NO: 8 - DNA, 20 bp, Artificial Sequence
  Primer
tttcccatctagcctcctca

SEQ ID NO: 9 - DNA, 20 bp, Artificial Sequence
  Primer
cctgaagcaaaacaacgtca

SEQ ID NO: 10 - DNA, 20 bp, Artificial Sequence
  Primer
gggttccggtagaacatgaa

SEQ ID NO: 11 - DNA, 20 bp, Artificial Sequence
  Primer
accgaaggctctgaagtcac

SEQ ID NO: 12 - DNA, 20 bp, Artificial Sequence
  Primer
ggggatggattcagtgaaga

SEQ ID NO: 13 - DNA, 20 bp, Artificial Sequence
  Primer
cttttccctgggtttccttc

SEQ ID NO: 14 - DNA, 20 bp, Artificial Sequence
  Primer
tccctctcaaccgactcaac

SEQ ID NO: 15 - DNA, 20 bp, Artificial Sequence
  Primer
tgactgcaatattgggcaaa

SEQ ID NO: 16 - DNA, 20 bp, Artificial Sequence
  Primer
aactttctgttcggctcacc

SEQ ID NO: 17 - DNA, 20 bp, Artificial Sequence
  Primer
gccatgggtgcttaccatag

SEQ ID NO: 18 - DNA, 20 bp, Artificial Sequence
  Primer
ccaagcctcgttttggttat

SEQ ID NO: 19 - DNA, 20 bp, Artificial Sequence
  Primer
cgatcttcccaaatgctgat

SEQ ID NO: 20 - DNA, 20 bp, Artificial Sequence
  Primer
gtccaggtcagctaggaacg

SEQ ID NO: 21 - DNA, 20 bp, Artificial Sequence
  Primer
gcgtgagctagagggagatg

SEQ ID NO: 22 - DNA, 20 bp, Artificial Sequence
  Primer
cagccagcgaacaaacacta

SEQ ID NO: 23 - DNA, 20 bp, Artificial Sequence
  Primer
tgcatgtacagccccattta

SEQ ID NO: 24 - DNA, 20 bp, Artificial Sequence
  Primer
gcagaacaggacgtgaaaca

SEQ ID NO: 25 - DNA, 20 bp, Artificial Sequence
  Primer
aggcctgtctgaacaccaat

SEQ ID NO: 26 - DNA, 20 bp, Artificial Sequence
  Primer
catgggcacagttgtagtgg

SEQ ID NO: 27 - DNA, 20 bp, Artificial Sequence
  Primer
cactcatcattctcggctca

SEQ ID NO: 28 - DNA, 20 bp, Artificial Sequence
  Primer
cacactatggactccgctca

SEQ ID NO: 29 - DNA, 30 bp, Artificial Sequence
  Primer
gtaatatactgacgccaaaagaggcggatt

SEQ ID NO: 30 - DNA, 26 bp, Artificial Sequence
  Primer
tcaactgctgttgtcgaggacattgg

SEQ ID NO: 31 - DNA, 31 bp, Artificial Sequence
  Primer
tgtaatatactgacgccaaaagaggcgactt

SEQ ID NO: 32 - DNA, 26 bp, Artificial Sequence
  Primer
tcaactgctgttgtcgaggacattgg

SEQ ID NO: 33 - DNA, 24 bp, Artificial Sequence
  Primer
caatcctgctccccaatccagacc

SEQ ID NO: 34 - DNA, 29 bp, Artificial Sequence
  Primer
gattacgagatcagcggtctggaaagaaa

SEQ ID NO: 35 - DNA, 25 bp, Artificial Sequence
  Primer
gcaatcctgctccccaatccagact

SEQ ID NO: 36 - DNA, 29 bp, Artificial Sequence
  Primer
gattacgagatcagcggtctggaaagaaa

SEQ ID NO: 37 - DNA, 26 bp, Artificial Sequence
  Primer
tggagcaatacgagggctactccaaa

SEQ ID NO: 38 - DNA, 28 bp, Artificial Sequence
  Primer
aatcttcagaaacgctccatttgtgctg

SEQ ID NO: 39 - DNA, 26 bp, Artificial Sequence
  Primer
tggagcaatacgagggctactccatg

SEQ ID NO: 40 - DNA, 28 bp, Artificial Sequence
  Primer
aatcttcagaaacgctccatttgtgctg

SEQ ID NO: 41 - DNA, 29 bp, Artificial Sequence
  Primer
tgtggtacaggtacacaagcgagaacatg

SEQ ID NO: 42 - DNA, 36 bp, Artificial Sequence
  Primer
ccttacaggcataacgagtatgagagattcataaca

SEQ ID NO: 43 - DNA, 35 bp, Artificial Sequence
  Primer
cttatttgtggtacaggtacacaagcgagaataaa

SEQ ID NO: 44 - DNA, 36 bp, Artificial Sequence; identical to SEQ ID NO: 42
  Primer

SEQ ID NO: 45 - DNA, 32 bp, Artificial Sequence
  Primer
gaagcattctttccgatacaatatggcctatc

SEQ ID NO: 46 - DNA, 32 bp, Artificial Sequence
  Primer
ttcgattaaaggattgttgatgaaactagggg

SEQ ID NO: 47 - DNA, 32 bp, Artificial Sequence
  Primer
gaagcattctttccgatacaatatggcctact

SEQ ID NO: 48 - DNA, 32 bp, Artificial Sequence; identical to SEQ ID NO: 46
  Primer

SEQ ID NO: 49 - DNA, 30 bp, Artificial Sequence
  Primer
ccataaatgtcattgtggagacatccgttc

SEQ ID NO: 50 - DNA, 25 bp, Artificial Sequence
  Primer
tggaacgtcaaaacattgaccggaa

SEQ ID NO: 51 - DNA, 26 bp, Artificial Sequence
  Primer
aaatgtcattgtggagacatccgggt

SEQ ID NO: 52 - DNA, 25 bp, Artificial Sequence
  Primer
tggaacgtcaaaacattgaccggaa

SEQ ID NO: 53 - DNA, 25 bp, Artificial Sequence
  Primer
ggtcatcggtgatggtggagaacct

SEQ ID NO: 54 - DNA, 27 bp, Artificial Sequence
  Primer
gggaattcgattatgtccatcacaccc

SEQ ID NO: 55 - DNA, 26 bp, Artificial Sequence
  Primer
aggtcatcggtgatggtggagatctg

SEQ ID NO: 56 - DNA, 27 bp, Artificial Sequence
  Primer
gggaattcgattatgtccatcacaccc

SEQ ID NO: 57 - DNA, 32 bp, Artificial Sequence
  Primer
cgaacccaacaacctgtaacaataagcactac

SEQ ID NO: 58 - DNA, 29 bp, Artificial Sequence
  Primer
ggaattcgattatctcggggctcatctac

SEQ ID NO: 59 - DNA, 31 bp, Artificial Sequence
  Primer
gaacccaacaacctgtaacaataagcagaaa

SEQ ID NO: 60 - DNA, 29 bp, Artificial Sequence
  Primer
ggaattcgattatctcggggctcatctac

SEQ ID NO: 61 - DNA, 24 bp, Artificial Sequence
  Primer
cacaagcgacttgaaactgcgctg

SEQ ID NO: 62 - DNA, 26 bp, Artificial Sequence
  Primer
ggcttgacaactgcttcaacctctgc

SEQ ID NO: 63 - DNA, 24 bp, Artificial Sequence
  Primer
cacaagcgacttgaaactgcaccc

SEQ ID NO: 64 - DNA, 26 bp, Artificial Sequence
  Primer
ggcttgacaactgcttcaacctctgc

SEQ ID NO: 65 - DNA, 29 bp, Artificial Sequence
  Primer
cagttctccaatcctttcctctgtggtct

SEQ ID NO: 66 - DNA, 25 bp, Artificial Sequence
  Primer
gtgagaagcgtgggatgctcatcag

SEQ ID NO: 67 - DNA, 27 bp, Artificial Sequence
  Primer
gttctccaatcctttcctctgtggtcg

SEQ ID NO: 68 - DNA, 25 bp, Artificial Sequence
  Primer
gtgagaagcgtgggatgctcatcag

SEQ ID NO: 69 - DNA, 27 bp, Artificial Sequence
  Primer
cagaggaagcccttacacagatccgac

SEQ ID NO: 70 - DNA, 25 bp, Artificial Sequence
  Primer
tacccacaggtctggaaagggcaag

SEQ ID NO: 71 - DNA, 27 bp, Artificial Sequence
  Primer
cagaggaagcccttacacagatccgat

SEQ ID NO: 72 - DNA, 25 bp, Artificial Sequence
  Primer
tacccacaggtctggaaagggcaag

SEQ ID NO: 73 - DNA, 25 bp, Artificial Sequence
  Primer
gcagtggaaggacatcattgcccat

SEQ ID NO: 74 - DNA, 21 bp, Artificial Sequence
  Primer
ctcttccgggacgcgacgttc

SEQ ID NO: 75 - DNA, 24 bp, Artificial Sequence
  Primer
cagtggaaggacatcattgccgtc

SEQ ID NO: 76 - DNA, 21 bp, Artificial Sequence
  Primer
ctcttccgggacgcgacgttc

SEQ ID NO: 77 - DNA, 21 bp, Artificial Sequence
  Primer
gcagcaccgtcaccggcacta

SEQ ID NO: 78 - DNA, 25 bp, Artificial Sequence
  Primer
gaggctcaatcaagatcgtctgccc

SEQ ID NO: 79 - DNA, 20 bp, Artificial Sequence
  Primer
cagcaccgtcaccggcatcg

SEQ ID NO: 80 - DNA, 25 bp, Artificial Sequence
  Primer
gaggctcaatcaagatcgtctgccc

SEQ ID NO: 81 - DNA, 31 bp, Artificial Sequence
  Primer
ctactctgagatcatcaacgagagcgtgaac

SEQ ID NO: 82 - DNA, 22 bp, Artificial Sequence
  Primer
cctagatcccaggcgagccgtc

SEQ ID NO: 83 - DNA, 31 bp, Artificial Sequence
  Primer
ctactctgagatcatcaacgagagcgtgttt

SEQ ID NO: 84 - DNA, 22 bp, Artificial Sequence
  Primer
cctagatcccaggcgagccgtc

SEQ ID NO: 85 - DNA, 25 bp, Artificial Sequence
  Primer
tcgagagcgatgccttctgacattg

SEQ ID NO: 86 - DNA, 29 bp, Artificial Sequence
  Primer
ccatatctccagccatcttcaatgttgtg

SEQ ID NO: 87 - DNA, 24 bp, Artificial Sequence
  Primer
cgagagcgatgccttctgacagca

SEQ ID NO: 88 - DNA, 29 bp, Artificial Sequence
  Primer
ccatatctccagccatcttcaatgttgtg

SEQ ID NO: 89 - DNA, 28 bp, Artificial Sequence
  Primer
atagaaggcagaatgaacgctggaaagc

SEQ ID NO: 90 - DNA, 22 bp, Artificial Sequence
  Primer
gggcaagcaggcctggaacttc

SEQ ID NO: 91 - DNA, 26 bp, Artificial Sequence
  Primer
agaaggcagaatgaacgctggactga

SEQ ID NO: 92 - DNA, 22 bp, Artificial Sequence
  Primer
gggcaagcaggcctggaacttc

SEQ ID NO: 93 - DNA, 31 bp, Artificial Sequence
  Primer
gaactacagacatgcacaaggatagcaggtt

SEQ ID NO: 94 - DNA, 24 bp, Artificial Sequence
  Primer
attgcattcaggaagctcgctcga

SEQ ID NO: 95 - DNA, 31 bp, Artificial Sequence
  Primer
gaactacagacatgcacaaggatagcagagc

SEQ ID NO: 96 - DNA, 24 bp, Artificial Sequence
  Primer
attgcattcaggaagctcgctcga

SEQ ID NO: 97 - DNA, 25 bp, Artificial Sequence
  Primer
cttcaatccgacaaccaagtcgctg

SEQ ID NO: 98 - DNA, 22 bp, Artificial Sequence
  Primer
ctggaactgcaatgcggccatt

SEQ ID NO: 99 - DNA, 26 bp, Artificial Sequence
  Primer
gcttcaatccgacaaccaagtcgcta

SEQ ID NO: 100 - DNA, 22 bp, Artificial Sequence
  Primer
ctggaactgcaatgcggccatt

SEQ ID NO: 101 - DNA, 28 bp, Artificial Sequence
  Primer
ttacggtgtccaatnnnnnnnnnnnnnt

SEQ ID NO: 102 - DNA, 28 bp, Artificial Sequence
  Primer
ttacggtgtccaaccgttcgtgccttct

SEQ ID NO: 103 - DNA, 28 bp, Artificial Sequence
  Primer
nnnnnnnnnnnnnccgttcgtgccttct

SEQ ID NO: 104 - DNA, 25 bp, Artificial Sequence
  Primer
gtcaccggcaccaaagaagctaata

SEQ ID NO: 105 - DNA, 25 bp, Artificial Sequence
  Primer
gtcaccggcaccgaagaagctaata

SEQ ID NO: 106 - DNA, 25 bp, Artificial Sequence
  Primer
agnnnnntnnnngnnnnnnnnnnnn

SEQ ID NO: 107 - DNA, 24 bp, Artificial Sequence
  Primer
cgagaatatgaagaaagcacccat

SEQ ID NO: 108 - DNA, 24 bp, Artificial Sequence
  Primer
cgagaatataaagaaagcacccat

SEQ ID NO: 109 - DNA, 24 bp, Artificial Sequence
  Primer
nnnnnnnnngnnnnnnnnnnnnnn

SEQ ID NO: 110 - DNA, 120 bp, Artificial Sequence
  Primer
cgccgccgtcgtgtcgtaagttgctcatcgataccgccacagcgcagcctgcgcgctgccagtttcttaggtcaactgaattctgaaaaacttctccgtctctaacctcagaatgaagga

SEQ ID NO: 111 - DNA, 20 bp, Artificial Sequence
  Primer
agggctactccagaatgtga

SEQ ID NO: 112 - DNA, 20 bp, Artificial Sequence
  Primer
agggctactccaggatgtga

SEQ ID NO: 113 - DNA, 20 bp, Artificial Sequence
  Primer
nnnnnnnnnnnnngnnnnnn

SEQ ID NO: 114 - DNA, 41 bp, Artificial Sequence
  Primer
attcatggagcaatacgagggctactccagaatgtgaacaa

SEQ ID NO: 115 - DNA, 41 bp, Artificial Sequence
  Primer
attcatggagcaatacgagggctactccaggatgtgaacaa

SEQ ID NO: 116 - DNA, 100 bp, Artificial Sequence
  Primer
catcggccaactaagcatgtggtttgacatatactataccaataacatttaaccagcatattacagcaacggatagaacaaaatgtggcccctccattta

SEQ ID NO: 117 - DNA, 100 bp, Artificial Sequence
  Primer
catcggccaactaagcatttggtttgacatatactataccaataacatnnaaccagcatattacagcaacggacagaacaaaatgtggcccctcgattta

SEQ ID NO: 118 - DNA, 100 bp, Artificial Sequence
  Primer
ccatcggtacgaccagattacaccagattaacaccaaaaaatactaattaatacatgagtataaaaagtaagcaaccatcatccataagttcaggattat

SEQ ID NO: 119 - DNA, 100 bp, Artificial Sequence
  Primer
ccatcggtacgaccagattacacaagatgaacaccaaaaaatactaattaatacatgagtataaaaagtaagcaaccatcatccataagttcaggattat

SEQ ID NO: 120 - DNA, 35 bp, Artificial Sequence
  Primer
taaaaccaagttccgatgtccaaaaattgggctct

SEQ ID NO: 121 - DNA, 35 bp, Artificial Sequence
  Primer
taaaaccaagttccgatgtccaaaatttgggctct

(19) United States
(12) Patent Application Publication
(10) Pub. No.: US 2015/0218571 A1
(43) Pub. Date: Aug. 6, 2015

(54) Compositions and Methods for Biofuel Crops

(71) Applicant: RUTGERS, THE STATE UNIVERSITY OF NEW JERSEY, New Brunswick, NJ (US)

(72) Inventors: Joachim Messing, Somerset, NJ (US); Calvino Torterolo, Highland Park, NJ (US); Remy Bruggman, New York, NY (US)

(21) Appl. No.: 14/565,269

(22) Filed: Dec. 9, 2014

Related U.S. Application Data
(63) Continuation of application No. 13/003,465, filed on Apr. 6, 2011, now abandoned.
(60) Provisional application No. 61/079,949, filed on Jul. 11, 2008.

(51) Int. Cl. C12N 15/82; C12Q 1/68

(52) U.S. Cl. CPC C12N 15/827; C12N 15/8245; C12N 15/8261; C12Q 1/6895

(57) ABSTRACT

Using the natural variation of sweet and grain sorghum to uncover genes that are conserved in rice, sorghum, and sugarcane, but differently expressed in sweet versus grain sorghum by using a microarray platform and the syntenous alignment of rice and sorghum genomic regions containing these genes. Indeed, enzymes involved in carbohydrate accumulation and those that reduce lignocellulose can be identified. Interestingly, C4 photosynthesis is enhanced as well. Furthermore, genetic analysis has shown that a specific microRNA is linked to flowering time and high sugar content in stems.

### CROSS-REFERENCE TO RELATED APPLICATIONS

This application is a continuation of U.S. patent application Ser. No. 13/003,465 filed Apr. 6, 2011, now abandoned, which is a §371 application of PCT/US09/50421 filed Jul. 13, 2009 which in turn claims priority to U.S. Provisional Patent Application No. 61/079,949 filed on Jul. 11, 2008, the disclosure of which is hereby incorporated by reference in its entirety.

### FIELD OF THE INVENTION

The present invention relates to compositions and methods to increase the sugar content and/or decrease the lignocellulose content in plants such as corn, rice, sorghum, Brachypodium, Miscanthus and switchgrass. The invention involves identifying genes responsible for sugar and lignocellulose production and genetically altering the plants to produce biofuels in non-food plants as well as the non-food portions of food crop plants to use as biofuel.

### BACKGROUND OF THE INVENTION

Energy from biomass has become attractive because of increased oil prices. However, current sources of biofuel have served as food and there are supply issues between these conflicting uses for these materials. Comparisons of genetic maps and sequences of several grass species have shown that there is global conservation of gene content and order (Gale and Devos, 1998). Therefore, grasses have been considered as a “single genetic system” (Bennetzen and Freeling, 1993). The practical aspect of such a concept is of great importance for agronomical purposes because a useful trait in one species could be transferred to another. A relevant example could be carbohydrate partitioning and allocation. In cereals such as wheat, corn, sorghum, and rice, the process of grain filling demands carbon from photosynthesis assimilation as well as the remobilization of pre-stored carbohydrates in the stem before and after anthesis (Yang and Zhang, 2006). It has been estimated that about 30% of the total yield in rice depends on the carbohydrate content accumulated in the stem before heading (Ishimaru et al., 2007). For these reasons, characterization of genes involved in carbohydrate metabolism and accumulation can lead to the development of improved crops.

In recent years there has been an increasing demand on biomass for the production of ethanol as a renewable resource for fuel. The biggest producers of ethanol in the world are Brazil and the United States (Ragauskas et al., 2006). In Brazil it is derived from sugarcane, while in the United States ethanol is derived from the grain of corn. Because of the use of the entire plant as a source for fermentable sugars, carbohydrate accumulation and partitioning has been extensively studied in sugarcane, probably more than in any other species (Ming et al., 2001). However, genes involved in these processes cannot easily be identified because of the complex genome of sugarcane, with several cultivars differing greatly in their ploidy levels from 2n=100 to 2n=130 chromosomes (D'Hont et al., 1996; Grivet and Arruda, 2002). Even if one could make further improvements to sugarcane, it has the disadvantage of being a crop restricted to tropical growing areas.

On the other hand, the use of corn grain for ethanol production poses a major conflict because of its dual use as food and fuel. Therefore, it has been proposed to use grain solely for food and only the stover as a source for ethanol. A major impediment to this approach is that in contrast to sugarcane, corn stover consists mainly of lignocellulose, which is more costly to process than fermentable sugars (Chapple and Carpita, 1998). Therefore, it would be attractive to identify corn varieties with reduced lignocellulose. Interestingly, there is extensive natural intra-species variation for sugar content in sorghum with cultivars that do not accumulate sugars (referred to as grain sorghums) in contrast to those that accumulate large amounts of sugars in their stems (Hoffman-Thoma et al., 1996). Such intra-species variation can serve as a platform to identify genes linked to increased sugar content and reduced lignocellulose. Moreover, if these genes are conserved by ancestry in related species, one could envision the introduction of such a trait by the import of specific regulatory regions. Conservation of gene order between closely related species permits the alignment of orthologous chromosomal segments. Non-collinear genes would constitute paralogous copies (Messing and Bennetzen, 2008). To facilitate such alignments, the use of rice with one of the smallest cereal genomes that has been sequenced (International Rice Genome Sequencing, 2005) increasingly becomes the anchor genome for other grasses (Messing and Llaca, 1998). In this sense, we can use rice as a reference genome for biofuel crops such as sugarcane and sorghum.

While rice offers an excellent reference as a compact genome from an evolutionary point of view, it is less suitable as a reference for a phenotype of reduced lignocellulose. Moreover, rice is a bambusoid C3 cereal plant and sorghum and sugarcane are panicoid C4 cereal plants, which branched out 50 mya (Kellogg, 2001). Sorghum and sugarcane belong to the Saccharinae clade and diverged from each other only 8-9 mya (Guimaraes et al., 1997; Jannoo et al., 2007). Therefore, sugarcane and its reduced lignocellulose can serve as a trait reference for sorghum varieties that differ in the cellulose content of their stems.

### SUMMARY OF THE INVENTION

The present invention is drawn to compositions and methods for adapting non-food plants as well as the non-food portions of current food crop plants to use as biofuel.

We have used microarray technology to compare genes expressed in the stem of sweet and grain sorghum. We have discovered 154 genes that were either up or down regulated in sweet sorghum. Computational analysis has shown that the differentially expressed genes are involved in starch and sucrose metabolism, sugar binding, enhanced C4 photosynthesis, and cell wall-related functions including cellulose fiber and lignin deposition. The regulation of these genes could be used to engineer crops or future crop species like switchgrass to have reduced lignocellulose. Reduction of lignocellulose in biofuel crops reduces the cost of extracting carbon from biomass for biofuel production as has been demonstrated with sugarcane in Brazil. However, sugarcane is a tropical C4 plant that cannot be grown in other climates like the US.

Currently, biofuel is derived from the grain of corn because grain is readily converted into bioethanol. Unlike sugarcane, the stem or stover of corn is high in lignocellulose rather than fermentable sugar. Therefore, corn stover remains untapped for bioethanol conversion. Introducing the trait from sweet sorghum in corn would facilitate the use of corn stover for bioethanol conversion without requiring increased production acreage.

Although sorghum like maize grain is used for the production of animal feed, it has a lower yield than maize. However, sorghum has a higher tolerance to drought and disease and could grow on rather marginal land. Therefore, sorghum itself has become an attractive biofuel crop. Because of the sweet sorghum cultivars that already exist, sweet sorghum could rival biofuel yields of sugarcane. Furthermore, identification of biofuel traits in sorghum could also be used to further enhance biofuel production from sorghum itself.

Key to the identification of and their regulatory elements the master regulators of the genes that we have discovered is a segregating population of sweet and grain sorghum. Such mapped sorghum sequences can be transferred in their original or modified form into maize or any other cereal genome by standard DNA transformation techniques (Frame, Bronwyn R, Shou, Huixia, Chikwamba, Rachel K, Zhang, Zhanyuan, Xiang, Chengbin, Fonger, Tina M, Pegg, Sue E, Li, Baochun, Nettleton, Dan S, Pei, Deqing, Wang, Kan. Agrobacterium tumefaciens-mediated transformation of maize embryos using a standard binary vector system. Plant Physiol. 2002 vol. 129 (1) pp. 13-22) (Wang, Kan, Frame, Bronwyn. Biolistic gun-mediated maize genetic transformation. Methods Mol Biol 2009 vol. 526 pp. 29-45) (and references therein) and the sugar content measured in modified plants using standard techniques described below.

The invention is described more fully herein. All references cited are hereby incorporated by reference in their entirety herein.

It is an object of the present invention to provide a genetically engineered plant comprising a selection of genes and their regulatory elements selected from the group consisting of: one or more genes differentially expressed between grain sorghum and sweet sorghum as provided in table 1, one or more genes in table 2, one or more genes in supplemental table 1, and one or more genes in supplemental table 2, that does not have the selection in nature, such that the genetically engineered plant provides for improved yield of biofuel production compared to a plant of the same species occurring in nature, and such that the genetically engineered plant (i) provides for increased sugar production as compared to the naturally occurring plant; or (ii) decreased lignocellulose production; or (iii) both (i) and (ii). In certain other embodiments, the selection of one or more genes is responsible for modifying starch and sucrose metabolism by effecting one or more enzymes selected from the group consisting of Hexokinase-8, carbohydrate phosphorylase, sucrose synthase 2, fructokinase-2 and sorbitol dehydrogenase. In certain other embodiments, the selection of one or more genes is responsible for modifying sugar binding by effecting D-mannose binding lectin. In certain other embodiments, the selection of one or more genes is responsible for carbon dioxide assimilation by effecting one or more NADP dependent malic enzymes.

In accordance with the above object, the invention is further directed to a genetically engineered plant wherein the selection of one or more genes is responsible for modifying cell wall properties by effecting one or more processes selected from the group consisting of LysM, cellulose synthase-7, cellulose synthase-1, cellulose synthase-9, cellulose synthase catalytic subunit 12, alpha-galactosidase precursor, beta-galactosidase 3 precursor, cinnamoyl CoA reductase, laccase, 4-Coumarate coenzyme A ligase, fasciclin domain, fasciclin-like protein FLA15, caffeoyl-CoA-methyltransferase 2, caffeoyl-CoA-methyltransferase, and caffeoyl-CoA O-methyltransferase. In certain other embodiments, the selection of one or more genes is responsible for modifying cell wall properties by effecting one or more processes selected from the group consisting of cinnamyl alcohol dehydrogenase, dolichyl-diphospho-oligosaccharide, xyloglucan endo-transglycosylase/hydrolase, putative xylanase inhibitor, glycosidase hydrolase family 1, phenylalanine ammonia-lyase, histadine ammonia-lyase, peroxidase and a process similar to Saposin type B protein. In still other embodiments, the biphosphate aldolase gene is used to increase sugar accumulation in the stem. In certain other embodiments, microRNA 172 (mi172) is used to increase sugar accumulation in the stem.

In accordance with any of the above objects, the invention is further directed to a genetically engineered plant wherein the selection of one or more genes has an orthologous copy in a syntenic position in rice.

In accordance with any of the above objects, the invention is further directed to a genetically engineered plant wherein the selection of one or more genes has a paralogous copy either in tandem or unlinked position relative to its orthologous donor copy.

In certain other embodiments, the amount of one or more soluble sugars selected from the group consisting of sucrose, glucose and fructose, is higher in the stem of the plant relative to a plant of the same species that does not that have the selection of one or more genes. In certain other embodiments, the plant provides for increased sugar production as compared to the naturally occurring plant.

In certain other embodiments, the plant provides for decreased lignocellulose production as compared to the naturally occurring plant.

In certain other embodiments, the plant provides for increased sugar production as compared to the naturally occurring plant and decreased lignocellulose production as compared to the naturally occurring plant.

In certain embodiments, the plant is selected from the group consisting of grain sorghum, sweet sorghum, maize, rice, Brachypodium, Miscanthus and switchgrass.

In certain embodiments, the invention is also directed to a method of developing plant cultivars to improve sugar content of a plant cultivar in geographic areas where there are short days comprising genetically engineering a plant cultivar with a short flowering time by including a selection of one ore more genes one or more genes differentially expressed between grain sorghum and sweet sorghum as provided in table 1, one or more genes in table 2, one or more genes in supplemental table 1, and one or more genes in supplemental table 2, wherein the plant cultivar does not have the selection in nature.

The invention is also directed to a method of developing plant cultivars adapted to different geographic areas by manipulating the flowering time to improve sugar content by including a selection of one ore more genes as set forth in any of the above embodiments.

The invention is also directed to a method of selecting a plant species having a sugar content above average comprising the correlation of the sugar content to the flowering time, determining the sugar content in late flowering plants is higher compared to early flowering plants, and selection and cultivation of late flowering plants. In certain other embodiments, the cultivar is grain sorghum. In certain other embodiments, the cultivar is sweet sorghum. In certain embodiments, the cultivar is a hybridized cultivar of grain sorghum and sweet sorghum. In certain embodiments, the cultivar is an F2 hybridized cultivar of grain sorghum and sweet sorghum.

In certain embodiments, in accordance with any of the above methods, the plant is Brachypodium.

In certain embodiments, in accordance with any of the above methods, the plant is Miscanthus.

In certain embodiments, in accordance with any of the above methods, the plant is switchgrass.

In certain embodiments, in accordance with any of the above methods, the plant is maize.

The invention is also directed to a method of increasing the sugar to lignocellulose ratio in a genetically engineered plant comprising a selection of genes and their regulatory elements selected from the group consisting of one or more genes differentially expressed between grain sorghum and sweet sorghum as provided in table 1, one or more genes in table 2, one or more genes in supplemental table 1, and one or more genes in supplemental table 2, that does not have the selection in nature, such that the genetically engineered plant provides for improved yield of biofuel production compared to a plant of the same species occurring in nature, and such that the genetically engineered plant (i) provides for increased sugar production as compared to the naturally occurring plant; or (ii) decreased lignocellulose production; or (iii) both (i) and (ii). In certain other embodiments, the invention is directed to a plant produced according to any of the methods set forth herein.

The invention is also directed to a genetically engineered plant comprising a selection of genes and their regulatory elements selected from the group consisting of: one or more genes differentially expressed between grain sorghum and sweet sorghum as provided in table 1, one or more genes in table 2, one or more genes in supplemental table 1, and one or more genes in supplemental table 2, that does not have the selection in nature, such that the genetically engineered plant provides for improved yield of biofuel production compared to a plant of the same species occurring in nature, and such that the genetically engineered plant (i) provides for increased sugar production as compared to the naturally occurring plant; or (ii) decreased lignocellulose production; or (iii) both (i) and (ii), wherein the regulatory elements comprise mi172. In certain other embodiments, the mi172 is mi172a. In certain other embodiments, the mi172 is mi172c. In certain other embodiments, the mi172 comprises mi172a and mi172c.

In certain other embodiments, the invention is directed to a method of increasing the sugar to lignocellulose ratio in a genetically engineered plant comprising a selection of genes and their regulatory elements selected from the group consisting of one or more genes differentially expressed between grain sorghum and sweet sorghum as provided in table 1, one or more genes in table 2, one or more genes in supplemental table 1, and one or more genes in supplemental table 2, that does not have the selection in nature, such that the genetically engineered plant provides for improved yield of biofuel production compared to a plant of the same species occurring in nature, and such that the genetically engineered plant (i) provides for increased sugar production as compared to the naturally occurring plant; or (ii) decreased lignocellulose production; or (iii) both (i) and (ii); wherein the regulatory elements comprise mi172. In certain other embodiments, the mi172 is mi172a. The method of claim 30, wherein the mi172 is mi172c. In certain other embodiments, the mi172 is mi172c. In certain other embodiments, the mi172 comprises mi172a and mi172c. In certain other embodiments, the invention is directed to a plant produced according to any of the above methods.

For purposes of the invention, the term “short days” means days having 10 hours of light and 14 hours of dark. The term “long days” means days having 16 hours of light and 8 hours of dark.

### BRIEF DESCRIPTION OF THE DRAWINGS

FIGS. 1A and 1B are graphical depictions of the variation in flowering time and Brix degree. FIG. 1A: Comparison of flowering time between grain sorghum Btx623 and six sweet sorghum genotypes. Time to flowering was measured as days required reaching 50% anthesis. FIG. 1B: Comparison of Brix degree along the main stem between grain sorghum Btx623 and 6 sweet sorghum genotypes. The Brix degree was measured for each internode and the average of a triplicate experiment was plotted.

FIGS. 2A, 2B, and 2C are graphical depictions of the validation of microarray data by semi-quantitative RT-PCR. FIG. 2A: The expression of Saposin type B, Starch phosphorylase, Beta-galactosidase 3 precursor, Sucrose synthase 2 and Cellulose synthase catalytic subunit 12 genes was analyzed by RT-PCR and agarose gel stained with ethidium bromide. The expression of Actin was used as a control. The results of three independent experiments for both BTx623 and Rio are shown. FIG. 2B: Quantification of the expression data shown in FIG. 2A. Results are presented as a proportion of the highest expression value for each gene between grain and sweet sorghum after standardization relative to Actin. FIG. 2C: RT-PCR comparing the expression of Saposin type B in BTx623 and two sweet sorghum lines Della and Dale.

FIG. 3 is a graphical depiction of the localization of differentially expressed genes on the physical map of sorghum. Each sugarcane probe set representing a differentially expressed gene between Btx623 and Rio with a fold change of 2 or higher was mapped to the sorghum genome and plotted on the physical map. Up-regulated genes are in red and down-regulated genes are in green.

FIG. 4 is a histogram showing the Brix degree at flowering time in BTx623, Rio and the F2 plants derived from the cross of these two cultivars. On the Y-axis is the number of plants and on the X-axis is the average Brix degree for three internodes of the main stem at flowering.

FIG. 5 is a histogram showing the flowering time, measured in numbers of leaves at the main stem, in BTx623, Rio and the F2 plants derived from the cross of these two cultivars. On the Y-axis is the number of plants and on the X-axis is the number of leaves at flowering.

FIG. 6 is a histogram showing the relationship between flowering time and Brix degree in BTx623, Rio and the F2 plants derived from the cross of these two cultivars. In the top graph, the Y-axis represents the Brix degree and the X-axis represents the number of leaves at flowering. In the bottom graph, the number of F2 plants with 9, 15 or 16 leaves at flowering are represented on the Y-axis whereas the average Brix degree for each F2 plants with 9, 15 and 16 leaves is represented on the X-axis.

FIG. 7 represents a set of histograms showing the average Brix degree of F2 plants differing in leaf number at the time of flowering.

FIG. 8 is a histogram showing the proportion of ELPs and SFPs between BTx623 and Rio for each sorghum chromosome. The number of genes with ELPs previously reported by Calviño et al. 2008 were plotted for each chromosome along with the number of SFPs found in this study. Only SFPs with t-values equal or greater than seven were considered.

FIGS. 9A and 9B are graphs showing the SFP discovery rate (SDR) of GeSNP is dependent on the t-value. The percentage of SFPs in sorghum genes that were validated through sequencing (and thus represented true single nucleotide polymorphisms (SNPs) between BTx623 and Rio) was plotted against their respective t-values (FIG. 9A). For the validated SFPs, we calculated the frequency distribution of their respective t-values (FIG. 9B).

FIGS. 10A-10D are graphical depictions of GeSNP prediction of SFPs in sorghum genes related to biofuel traits. The hybridization intensity between the perfect match (PM) and the mismatch (MM) oligonucleotides was averaged and scaled (GeSNP software output) and plotted against each sugarcane probe pair. Graphs are shown for four genes related to biofuel traits that have SFPs with t-values of seven or greater and that were previously reported to be differentially expressed between grain sorghum BTx623 and sweet sorghum Rio (FIG. 10A). The SFP present in lysM identified a 13 bp indel, whereas the SFPs present in cellulose synthase 1 and dolichyl-disphospho-oligosaccharide identified an A/G and G/A SNP between BTx623 and Rio respectively (FIG. 10B). Sequences in FIG. 10B are SEQ ID NOs: 101-109, from top to bottom. In Rio, the third intron of the gene 4-coumarate coenzyme A ligase is mis-spliced and detected in the sugarcane prope pair #2 (FIG. 10C). Sequence in FIG. 10C is SEQ ID NO: 110. Molecular markers for the genes lysM, cellulose synthase 1 and dolichyl-diphospho-oligosaccharide were generated based on allele-specific PCR. In the case of lysM, a primer spanning the 13 bp deletion in BTx623 was used to selectively amplify the allele from Rio. In the case of cellulose synthase 1 and dolichyl-diphospho-oligosaccharide, primer pairs specific for the SNP in question were generated by the WebSNAPER software and tested empirically (FIG. 10D).

FIGS. 11A and 11B are graphical depictions of SNP density per sorghum chromosomes. The number of SNPs per Kb of sequence was calculated based on the number of genes sequenced belonging to a given chromosome. Only those chromosomes with 5 or more genes sequenced are represented (FIG. 11A). Frequency distribution along sorghum chromosomes of sugarcane probe pairs with t-values between 22 and 25 (FIG. 11B).

FIGS. 12A, 12B, and 12C are graphical depictions of development of a molecular marker for alanine aminotransferase based on SFP discovery and the SNAP technique. The SFP detected by the probe pair #5 in the sugarcane probe set Sof.1326.1.S1_a_at was validated through sequencing (FIG. 12A). Sequences are SEQ ID NOs: 111-113, from top to bottom. Specific primers for either A or G nucleotides were designed with WebSNAPER (FIG. 12B) and tested through PCR in 10 sorghum lines (FIG. 12C). Sequences in FIG. 12B are SEQ ID NOs: 113-115, from top to bottom.

FIG. 13 is a graphical depiction of SFP validation for fructose bisphosphate aldolase. A fragment from the gene fructose bisphosphate aldolase was cloned and sequenced from both BTx623 and Rio and SNPs predicted by the probe pairs #8, 9 and 11 were validated. The blue lines represent the sugarcane probe pairs that are identical to either the Rio sequence (probe pairs #8 and #9) or identical to the BTx623 sequence (probe pair #11). Sequences are SEQ ID NOs: 116-121, from top to bottom.

FIG. 14 is a graphical depiction of the position of the SNP along the 25mer in the probe pair influences the SFP validation. The position of the SNP from the edge of the sugarcane probe pair was scored for each validated SFP. Most of the SNPs locate within positions 6 and 13 along the 25mer. If two or more SNPs were located on a single probe pair, their positions along the 25mer were not counted and thus not included in the graphs.

### DETAILED DESCRIPTION OF THE INVENTION

One objective of the present invention is to change the ratio of lignocellulose to sugar in feedstock using translational genomics, which would double the bioethanol output in grass species like Miscanthus and switchgrass. Miscanthus and switchgrass are low-input species that grow on non-arable land. If we were to replace the equivalent of arable land with non-arable land to grow improved Miscanthus and switchgrass, we could produce at least 16% of our current total transportation fuel at 42 cents per gallon with a greenhouse emission reduction of 50% over the use of gasoline only. To reach this goal, we would like to increase the fermentable sugar in suitable grass species to levels found in sugarcane (some cultivars up to 20 Brix degrees) by modifying the expression of key genes indentified in sweet sorghum through genetic engineering of target species. Because of its complex genome sugarcane is not suitable for identifying genes that control the ratio of sugar to lignocellulose. Moreover, there is no sugarcane variety available with low sugar and high lignocellulose content, which is necessary to use genetic linkage analysis to identify regulatory elements associated with our trait of interest in its genome. On the other hand, sorghum is closely related to sugarcane, has cultivars with high sugar content (sweet sorghum; 17-19 Brix degrees) and low sugar content (grain sorghum; 6-8 Brix degrees), and has a small completely sequenced genome.

As an example of how translational genomics could be implemented, one could use a three-tier approach. Sorghum would be the first tier model for identifying the genes that control sugar content. One could take advantage of our segregating population of sweet and grain sorghum to identify genes linked to high-sugar content and reduced lignocellulose content in the stem by positional cloning. Such an effort would also yield physically linked molecular markers (single nucleotide polymorphisms, SNPs) to these traits. Because interspecies crosses could be performed between sorghum and Miscanthus, these markers would also be used for introgression of sweet sorghum chromosomal intervals containing these genes into Miscanthus. The second tier could involve functional analysis of the candidate genes identified in sorghum in a model system like the grass Brachypodium, whose genome has also been sequenced. Due to its small size, rapid generation time, and highly efficient transformation one could rapidly evaluate many candidate genes, including small RNAs as potential key regulators, in Brachypodium. The third tier could be testing a subset of promising genes from the Brachypodium work in switchgrass. Therefore, one could 1) identify SNPs to develop molecular markers linked to high sugar content in the stem of sweet sorghum, 2) use this markers for the introgression of sorghum chromosomal intervals into Miscanthus 3) positionally clone genes linked to high sugar content in the stem of sorghum, 4) transform Brachypodium with candidate sorghum genes and measure sugar and lignin content in transgenic stems, and 5) increase the sugar content of switchgrass stems using the genes that maximized sugar content in Brachypodium stems.

Major challenges have arisen from the call to use biomass for the production of biofuels. Most carbohydrates accumulate in form of lignocellulose, which due to its recalcitrance to degradation is difficult to convert into liquid fuel. Therefore, sugarcane, which has a high percentage of fermentable sugar throughout the plant, and maize seeds, which are composed largely of starch, are the dominant feedstocks for biofuel production today. However, sugarcane is a tropical crop and does not grow in temperate climates and cornstarch is a major source for food, feed, and fiber products. Furthermore, they are high-input cultivated crops. Therefore, alternate species (e.g. switchgrass, Miscanthus) have been proposed as biofuel crops for temperate areas. The focus on using lignocellulosic biomass as feedstocks has created the need for developing less costly processes for breaking down lignocellulose in sugar monomers that can be fermented into biofuels. Considering such a need, one could incorporate the properties of sugarcane into biomass crops suited to temperate regions.

Alternatively, the same methods can be used to further improve sorghum as a biofuel crop. As shown below, sweet sorghum cultivars vary in stem sugar measured in Brix degree significantly, indicating that stem sugar in sweet sorghum could be further improved. Comparative analysis of sweet sorghum cultivars could be used to identify regulatory elements that lead to incremental higher levels of stem sugar in sweet sorghum cultivars with superior yield and other desirable traits like draught resistance and nitrogen efficiency use. Such an approach of combining desirable traits within the same species by DNA transformation techniques and conventional breeding is also referred to as “stacking.”

### Technical Approach/Work Plan

One approach would be the identification of genes that are expressed or repressed during sugarcane stem development, in order to design genetic modifications of target temperate species. There are two major problems of using sugarcane for these studies. First, sugarcane does not have a well-characterized variant high in lignocellulose (low in soluble sugars) that could serve as a reference. The second problem is that the complex sugar-cane genome has undergone several rounds of whole genome duplications in recent times and therefore not been sequenced. A more suitable system is the closely related species Sorghum bicolor, whose genome is much simpler than that of sugarcane. According to the common scientific consensus progenitors of sorghum and sugarcane split 8-9 million years ago (mya). Moreover, there are sorghum cultivars with high-sugar content in their stems (sweet sorghum) and low sugar content in their stems (grain sorghum). Sweet sorghum reaches Brix degrees of 17-19, although some sugarcane cultivars can reach a Brix degree of 20.

Furthermore, with DOE JGI support and in collaboration with the University of Georgia we have recently sequenced and annotated the genome of sorghum. DOE selected our project because of the potential of sorghum to serve as a model for biofuel crops. We also conducted microarray expression profiles between grain and sweet sorghum using a sugarcane array and discovered that sweet sorghum differentially expresses many of the genes previously reported to be involved in sugarcane stem growth. Actually, it appeared that the comparison between sorghum genotypes differing in sugar content was more sensitive to the discovery of differentially expressed genes than expression profiling of the same sugarcane genotype throughout different stages of stem development. Interestingly, when we mapped the sugarcane probe sets that feature differential expression to the grain sorghum genome sequence, we found that out of 154 genes 123 were collinear between sorghum and rice, indicating that these genes have been conserved over 50 million years. Because this time span predates the radiation of the grass family (60-70 mya), we assume that, in principle, the metabolic pathways are conserved at the DNA sequence level within all grasses and that translational genomics to introduce high-sugar stem traits has a high probability of succeeding. We seek to characterize the regulatory circuits that give rise to high sugar content in sweet sorghum so that a rational design could be used in other grass species to optimize their utilization as biofuel sources. Another useful feature of sorghum is the use of interspecific hybrids. For instance, marker selected introgressions using hybrids between sorghum and Miscanthus could be used to lower the lignocellulose content of Miscanthus in favor of fermentable sugars without any transgenic methods. Therefore, we are convinced that sorghum would be an excellent model system to study the genetic basis of sugar accumulation in the stem.

Another useful feature of interspecific hybrids between sorghum and Miscanthus could be the improvement of sorghum as a biofuel crop. Miscanthus is a perennial crop that is reproduced by cuttings and vegetative reproduction. Because its root system is thereby saved, it has adapted to high “nitrogen efficiency use.” On the other hand sorghum requires fertilizer for optimal production. If one could introduce genetic loci from Miscanthus controlling high “nitrogen efficiency use” into sorghum using molecular marker-assisted breeding, input and environmental cost of fertilizer use for growing sorghum as a biofuel crop could be reduced. Therefore, interspecific hybrids can be used for both species. In Miscanthus, one can lower lignocellulose in the stem and in sorghum one can lower production costs and reduce chemical run-offs to preserve water quality in production areas.

Although we found genes belonging to several metabolic pathways such as the starch and sucrose pathway together with cell wall-related and osmotic stress pathways that were differentially expressed in stems of sweet sorghum versus grain sorghum, we do not know the molecular basis of the regulatory circuits underlying the change in gene expression of such a diverse set of genes and networks. Answering such questions requires a genetic approach, where we test for the co-segregation of molecular markers in candidate genes related to high sugar content in a segregating population. We have already created F₂ mapping population derived from grain (Btx623) and sweet sorghum (Rio) and by applying the concept of bulk segregant analysis (BSA), we isolated those F2 plants differing in the sugar content of their stems (measured as Brix degree) by at least two fold. At the same time, we have been developing molecular markers based on SNPs for those genes differentially expressed between sweet and grain sorghum. Our preliminary data suggests that on average there is one SNP every 264 bp of sequence between BTx623 and Rio. Assuming that Rio has the same genome size as BTx623 (730 Mbp), this would give a minimum number of 2,766,199 SNPs between BTx623 and Rio genomes (only SNPs in exons or 3′UTRs were considered). Molecular markers could then be used for two applications: marker-assisted introgressions of sweet sorghum intervals into Miscanthus by regular breeding and the cloning of candidate genes by chromosomal positions using the genomic sequence of sorghum.

To obtain these molecular markers, we will apply SOLiD sequencing of the Rio genome and F₂ plants selected with bulk segregant analysis (BSA) to perform a genome wide screen of SNPs that co-segregate with high sugar content. We also plan SOLiD sequencing of the genomes of the sweet sorghum lines Simon, Top 76-6, M81-E, Della, and Dale, which differ in their Brix degrees in stem tissue and flowering times. Natural variations have the potential to uncover different quantitative traits. As discussed above, regulatory elements that provide incremental levels of stem sugar could be modified to further increase the stem sugar also in sweet sorghum. Because we already have the sequence of the Btx623 line available at high accuracy, we can resequence the sweet sorghum lines using our new SOLiD version 3 next generation sequencing system and map these sequences back to the sequenced reference genome. A crucial point in this process is the use of mate pairs by sequencing the ends of sheared libraries created with different but uniform sequence lengths. These mate pair reads allow us to anchor sequences by physical linkage and distance within a genome containing repeat sequences. Currently, we sequence 20 Gb per run, but we expect a two-fold higher throughput at the same price with the recent upgrade. At this stage, for $10,000, we could produce 57-fold sequence coverage per cultivar (two insert sizes and paired reads of 50 bp), providing sufficient sequence information to reliably determine SNPs for the identification of candidate genes for sugar content through BSA.

We also plan to expand our current expression database using the SOLiD system. We would perform expression profiling by sequencing cDNAs from grain and sweet sorghum. Furthermore, we already constructed small RNA libraries to add to our inventory of differentially expressed RNAs. The combination of genomics-based BSA and expression profiling will be used to identify candidate elements capable of regulating the carbohydrate-related metabolic pathways in sweet sorghum. To test their presumptive function, we could introduce candidate sequences into Brachypodium, which is also considered as a model for biofuel crops. There are technical and scientific reasons to use a heterologous system rather than sorghum for this part of the project. From a technical standpoint, Brachypodium offers tremendous advantages in terms of transformation efficiency (44% efficiency on average), the time required to create transgenics (we can generate transgenic lines in as little as 12 weeks). In addition, its small size and rapid generation time (8 weeks) will greatly accelerate downstream analysis of transgenic lines. For these reasons we would be able to test many genes and gene combinations using a transgenic approach. The Brachypodium genome is completely sequenced, which will greatly facilitate the evaluation of the role of endogenous genes that will presumably be required to synthesize sugars in stems. A Brachypodium microarray will be available shortly (Todd Mockler pers. comm.) and this will be particularly useful in determining the effects of regulatory genes on global gene expression. From a scientific perspective, it makes sense to use a heterologous system because our ultimate goal is to introduce high sugar stem traits into other biomass crops like switchgrass and Miscanthus. Thus, if we can develop an effective approach to increase stem sugar content in Brachypodium, it is likely that that approach will work with other grasses.

Once regulatory elements linked to the high stem sugar in sweet sorghum have been identified, one can also modify those elements in sorghum to further enhance sugar accumulation in sweet sorghum. Clearly there is natural variation among sweet sorghum lines in respect to Brix degrees in their stems as shown by our analysis. Although conventional breeding is used to increase sugar accumulation in sweet sorghum cultivars, the identification of regulatory elements required for high Brix degrees and their introduction into sorghum cultivars by genetic engineering (Gurel, Songul, Gurel, Ekrem, Kaur, Rajvinder, Wong, Joshua, Meng, Ling, Tan, Han-Qi Q, Lemaux, Peggy G. Efficient, reproducible Agrobacterium-mediated transformation of sorghum using heat treatment of immature embryos. Plant Cell Rep 2009 vol. 28 (3) pp. 429-44) could further optimize sorghum as a biofuel crop.

### Energy Efficiency/Displacement, Rural Economic Development, and Environmental Benefits

The US currently imports 55% of its petroleum, which accounts for 45% of the total trade deficit. Decreasing our dependence on petroleum imports by developing new and existing sources of renewable energy will stimulate the economy, increase energy security, improve air quality through the use of ethanol as a fuel additive and decrease the quantity of CO2 and other greenhouse gases released into the atmosphere. According to Wikipedia, estimated greenhouse gas emission reduction because of the use of bioethanol as a fuel in Brazil is 86-90% and in the US only 10-30%. Therefore, biomass represents an underutilized renewable energy source with the potential to supply a significant portion of our fuel needs and a huge environmental benefit. Although sugarcane is hailed as the most efficient source of bioethanol, seven-times better than corn, it also, like corn, is a relatively high-input crop. Because of the low input of switchgrass we could improve this input/output by a factor of two, greatly boosting greenhouse gas emission reduction. Currently, Brazil's cost for a gallon of bioethanol is 84 cents (US $1.33, the difference is equalized with tariffs and subsidies). With lower input cost, we could reduce the cost to 42 cents per gallon. However, switchgrass has higher downstream costs because it consists mostly of lignocellulose. The differential output between sugarcane and corn is due to the fact that the stem of corn has mostly lignocellulose. Therefore, it appears that a factor of 7 for reduced lignocellulose and increased sugar in the stem could facilitate a greater yield of bioethanol per acre of switchgrass. Brazil produces currently 800 gallons of bioethanol/acre. If we could achieve such an amount with switchgrass with 42 cents a gallon, we could raise energy efficiency and environmental benefits simultaneously. Associated environmental benefits of switchgrass cultivation also derive from its large root mass that increases soil organic matter, prevents soil erosion, and acts as a carbon sink further reducing greenhouse gases. Switchgrass is planted in either pure stands or as a component of a mixture on a significant amount of the CRP land in the Great Plains and Midwest and is currently utilized as a pasture and range grass in mid-latitude states on land that is less suitable for cultivation of crops for human consumption. Last year, the U.S. used about 50 million acres or 6% of arable land for corn bioethanol, which provides about 13 billion gallons of ethanol or 8.2% of total fuel. Just by using the equivalent non-arable, much less valuable land for switchgrass, we could double our output on bioethanol to 16% of total fuel for a lower price of 42 cents on land in rural areas where no other economic opportunity exists.

### Results

Sugar Accumulation in the Stem of Grain and Sweet Sorghum Cultivars

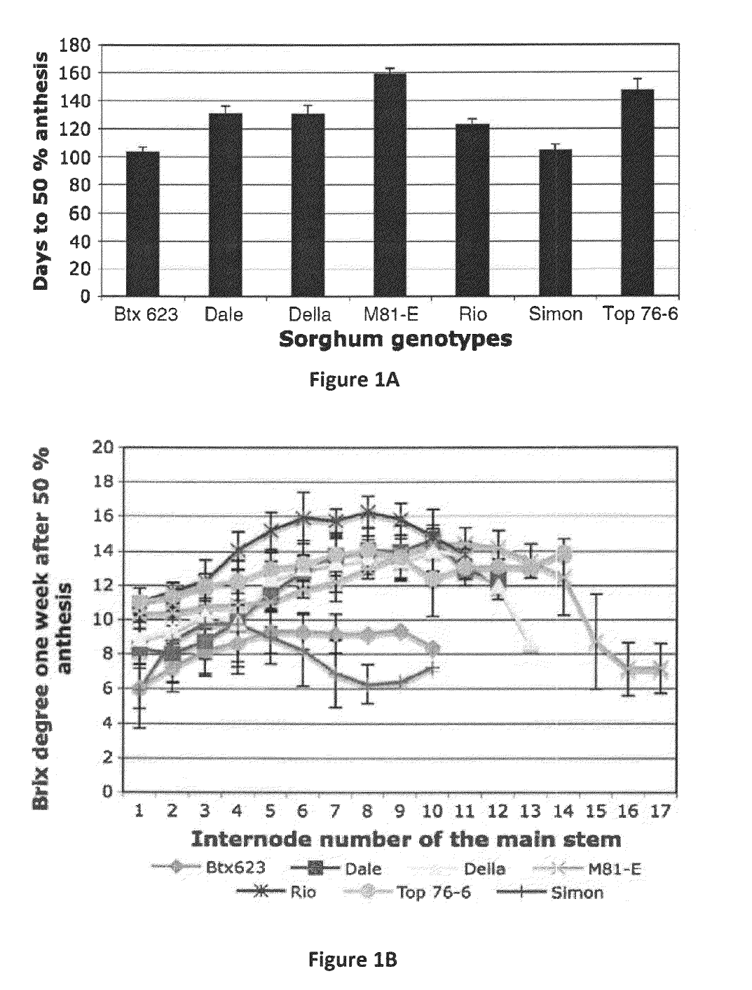

Previous reports have indicated that in sorghum stems, sugars start to accumulate at flowering stage (Lingle, 1987; Hoffman-Thoma et al., 1996). We compared the accumulation of sugars in the stem between six sweet sorghum lines (Dale, Della, M81-E, Rio, Top76-6 and Simon) and one line from grain sorghum (BTx623). As an estimation of the total amount of sugars present in the juice of sorghum stems, we measured the Brix degree of each internode along the main stem at the time of flowering. We found great variation in flowering time as well as in Brix degree between the sweet sorghum lines when compared to grain sorghum BTx623 (FIGS. 1A and B). In general, the Brix degree was lower in the mature and immature internodes of the stem, in contrast to maturing internodes. These findings are in agreement with previous studies (Lingle, 1987; Hoffman-Thoma et al., 1996). Consistent with the inability of grain sorghum to accumulate significant levels of sugars in the stem, the Brix degree in BTx623 was low and remained fairly constant for all the internodes along the stem. Among the sweet sorghum cultivars Rio had the highest Brix degree and Simon the lowest. Furthermore, the difference in flowering time between BTx623 and Rio was smaller than in the rest of sweet sorghum lines with high Brix degrees. For this reason, we decided to perform a comparative analysis of transcripts in the stem of the Rio and BTx623 sorghum lines.

Microarray Analysis of Transcripts from Sorghum Stem Tissues

In order to identify genes expressed in the stem with a potential role in sugar accumulation and reduced lignocellulose, we compared transcript profiles between grain (BTx623) and sweet sorghum (Rio). Such a genome-wide analysis became possible because of the recently designed GeneChip of sugarcane (Casu et al., 2007). This array was specifically developed with sequences that were obtained from several cDNA libraries representing distinct tissue types including stem, from 15 sugarcane varieties. The use of this GeneChip permitted us to directly compare gene expression data of two different sorghum cultivars with the previously generated data from sugarcane. Three independent plants for each BTx623 and Rio were grown until anthesis and RNA was extracted from the same maturing internode for all six plants. These RNAs were used to prepare biotylinated cRNAs for hybridization, each sample separately hybridized to one array.

The sugarcane array comprised a probe set of 8,224 oligonucleotides, of which more than 70% (5,900) gave a positive signal with sorghum RNA samples. When a two-fold cut-off value was applied as criterion to distinguish differentially expressed transcripts between grain and sweet sorghum, a total of 195 transcripts were identified, with 132 transcripts being up-regulated and 63 transcripts down-regulated in Rio, respectively (Supplemental table 1 and 2). Based on the annotation of the sorghum genes, we were able to infer the possible function for most of the differentially expressed transcripts.

Among the transcripts that were up regulated in Rio, a Saposin-like type B gene displayed the highest differential expression. SAPOSINS are involved in the degradation of sphingolipids (Munford et al., 1995). Other transcripts encoding stress related proteins such as HEAT SHOCK PROTEIN 70 (HSP70) and HSP90 were up regulated, consistent with an osmotic stress imposed by high concentration of sugars (Supplemental table 1 and 2). Our results show that in Rio, down-regulated genes outnumber those that are up regulated by a factor of 2. The most reduced transcript has a fasciclin domain. This domain has been shown to be involved in cell adhesion (Table 1) (Kawamoto et al., 1998; Faik et al., 2006).

Genes with Altered Expression in Carbohydrate Metabolism in Sweet Sorghum

Based on Gene Ontology (GO) terms (www.geneontology.org/), the sucrose and starch metabolic pathway from the Kyoto Encyclopedia of Genes and Genomes (KEGG) (www.genome.jp/kegg/), and the Carbohydrate-Active enzymes (CAZy) database (www.cazy.org/), we found that almost 16% of the transcripts that were differentially expressed between BTx623 and Rio, corresponded to transcripts affecting carbohydrate metabolism (Table 1 and 2). Within these, transcripts that were up regulated include hexokinase 8 and carbohydrate phosphorylase (starch and sucrose metabolism), NADP malic enzyme (C4 photosynthesis), a D-mannose binding lectin (sugar binding) and a LysM (Lysin Motif) domain protein possibly involved in cell wall degradation. Transcripts that were down regulated included sucrose synthase 2 and fructokinase 2 (starch and sucrose metabolism), alpha-galactosidase and beta-galactosidase (hydrolysis of glycosidic bonds) and cellulose synthase 1, 7, and 9 together with cellulose synthase catalytic subunit 12 (cell wall metabolism). In addition, several others transcripts with a cell wall-related role that were down-regulated included cinnamoyl CoA reductase, cinnamyl alcohol dehydrogenase, 4-coumarate coenzyme A ligase, caffeoyl-CoA O-methyltransferase, xyloglucan endo-transglycosylase/hydrolase, peroxidase and phenylalanine and histidine ammonia-lyase.

Validation of Microarray Data by RT-PCR

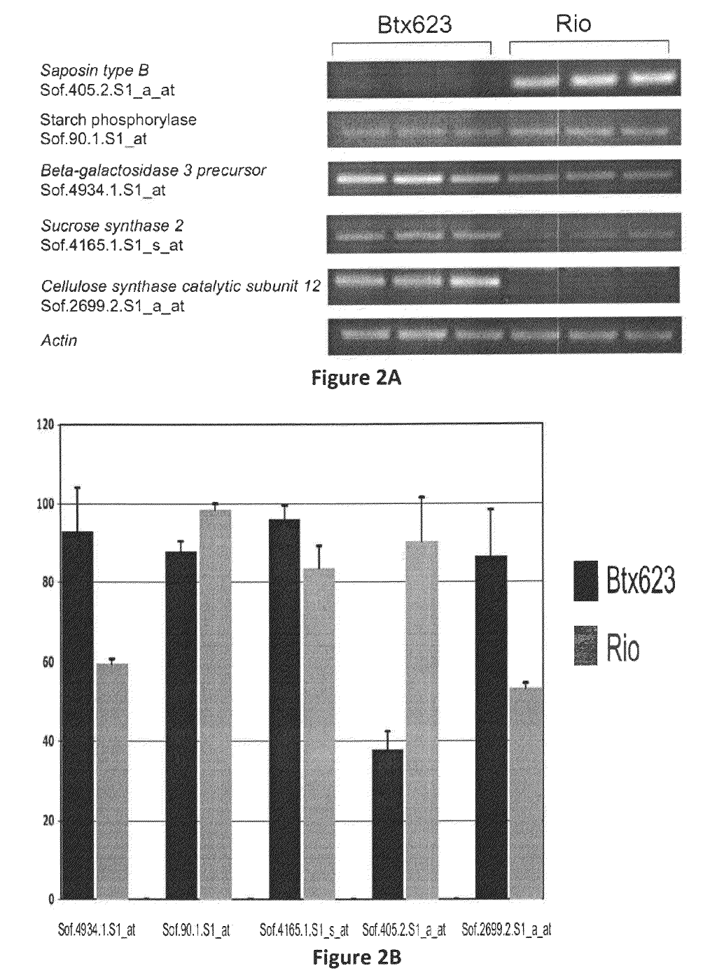

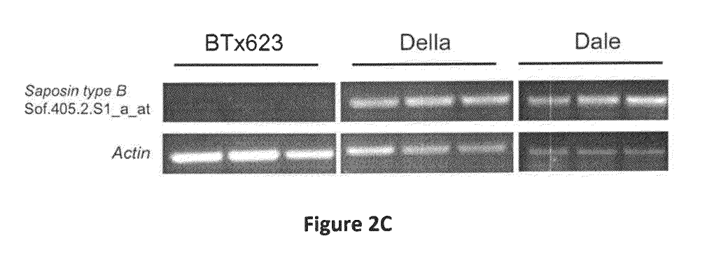

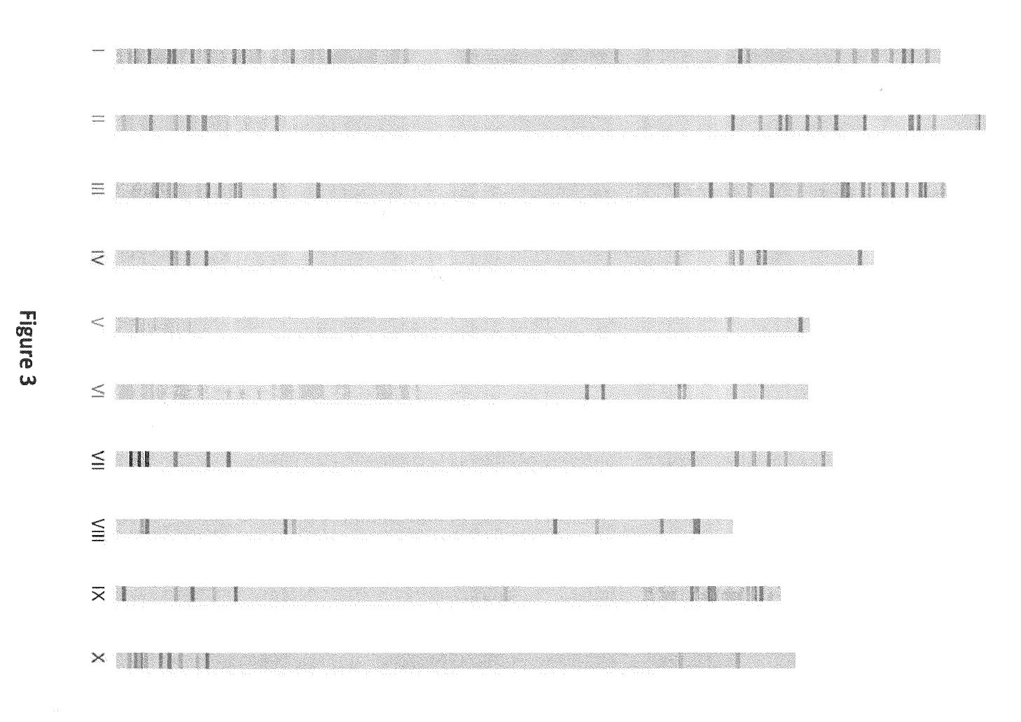

To validate the data obtained by microarray analysis, we selected five genes and compared their expression levels in both Rio and BTx623 by performing semi quantitative RT-PCR (FIGS. 2A and B). In Rio, the expression of Saposin and Carbohydrate Phosphorylase is up regulated in comparison with their expression in Btx623. In contrast, the expression of Beta-galactosidase 3, Sucrose Synthase 2 and Cellulose Synthase catalytic subunit 12 were down regulated in Rio. Thus, we can validate the microarray analysis with a different method. In order to see if the expression difference between BTx623 and Rio for the transcript encoding a SAPOSIN-type B protein also extended to other sweet sorghum lines, we extracted RNA from maturing stems of BTx623, Dale and Della at flowering and measured the expression of Saposin by RT-PCR. We found that this gene is also highly expressed in Dale and Della when compared to grain sorghum (FIG. 2C).

Genomic Location of Differentially Expressed Genes

In order to see if some of the genes that were differentially expressed between grain and sweet sorghum cluster together in a particular region of the sorghum genome, we generated a “transcriptome map” (FIG. 3). We mapped the sequences of all up and down regulated sugarcane probes to the recently sequenced Sorghum genome (BTx623) (www.phytozome.net/cgi-bin/gbrowse/sorghum/) using GenomeThreader (Gremme et al., 2005). From a total of 195 probe sets, 176 of them could be mapped to the sorghum genome based on their overlap with a sorghum gene (Materials and Methods). In addition, 6 probe sets could be mapped to the genome but do not overlap with the current sorghum gene annotation and for another 13 probe sets we were not able to map them to the sorghum genome. Genes that were differentially expressed between grain and sweet sorghum do not appear to cluster in any particular region of the genome but rather reflect random distribution (FIG. 3).

Trait-Specific Syntenic Gene Pairs Between Rice and Sorghum

It can be considered that important gene functions have been conserved by ancestry and that divergence is mainly due to changes in regulatory control regions of genes. To determine the ancestry of genes, however, requires the alignment of syntenic regions. Because we know now the positions of the sorghum genes in their respective chromosomes we can align them with the rice genome as a reference (International Rice Genome Sequencing, 2005) and determine whether the aligned regions are collinear between rice and sorghum. Indeed, we found that from a total of 158 sorghum genes, 123 have an orthologous copy in syntenic positions in rice. Interestingly, we found that sucrose synthase 2 is duplicated in rice but not in grain sorghum. So the question arose whether gene copy number would make a difference in expression levels between grain and sweet sorghum. Because we have only the sequence of grain sorghum, we performed a Southern blot analysis of genomic DNA of sweet sorghum. When genomic DNA from BTx623 and Rio are compared, both possess a single copy of sucrose synthase 2 (data not shown).

### Discussion

Translational Genomics

The non-renewable nature of fossil oil imposes an increasing pressure to develop alternatives energies in order to support and secure social and economic growth in the near future (Ragauskas et al., 2006). Currently, there is a worldwide interest to develop biofuel crops, the best example being sugarcane, used in Brazil since 1970s. Besides sugarcane, other grasses such as Brachypodium distachyon, Miscanthus, maize, rice, sweet sorghum and switchgrass are considered as crops for biofuel research and production. However, the challenge of combining multigenic traits of one species with the traits of another if traditional crosses are restricted to each species exists. Recently, the entire gene cluster of 10 sorghum kafirin genes contained within a chromosomal segment of 45 kb was intact and stably inserted into the maize genome. Expression analysis then has shown that kafirins accumulated in maize endosperm in a developmental and tissue specific manner (Song et al., 2004). Such transfer of genomic DNA between species that cannot be crossed could then be used in advanced breeding techniques to introduce desirable traits from one species to another. Here, we integrate the traits of sugar accumulation and lignocellulose content with genomic and expression data of the three species, sugarcane, sorghum, and rice. We used the recently developed Affymetrix sugarcane genome array (Casu et al., 2007) as a tool for the identification of genes differentially expressed in maturing stems of grain and sweet sorghum. The intra-species variation for sugar content in sorghum is more pronounced than between sugarcane varieties, making sorghum a more suitable model to study this trait. On the other hand, because we can map sorghum genes to their chromosomal positions, we can use rice as a reference genome to identify genes by their ancestry.

Cross-Referencing Tissue-Specific Transcripts

Sorghum and sugarcane belong to the Saccharinae clade and diverged from each other only 8 to 9 mya (Janoo et al. 2007), while rice is a more distant relative and separated from this clade 50 mya (Kellogg, 2001). Because sorghum and sugarcane belong to the same clade, we reasoned that by hybridizing RNA from grain and sweet sorghum onto the sugarcane GeneChip we could correlate changes in transcript levels with traits from sweet sorghum such as sugar content and reduced lignocellulose. Given the tissue-specificity and the rather small gene set of the sugarcane GeneChip, the positive hybridization of stem-derived RNAs from sorghum to 5,900 sugarcane probes of a GeneChip comprising 8,224 probe sets in total is a good indication of such cross-referencing. By applying a two-fold cut off value as a parameter to filter out differentially expressed transcripts, a total of 195 probe sets were identified, of which 63 corresponded to transcripts that were up regulated and 132 corresponded to transcripts that were down regulated in the sweet sorghum Rio line, respectively. Each differentially expressed sorghum transcript was classified based on the Pfam domains of their encoded proteins and their GO term (Materials and Methods).

Based on the sucrose and starch metabolic pathway from the Kyoto Encyclopedia of Genes and Genomes (KEGG) (www.genome.jp/kegg/) and the Carbohydrate-Active enzymes (CAZy) database (www.cazy.org/), we found that almost 16% of the transcripts involved in sucrose and starch metabolism and in cell wall related processes were differentially expressed between BTx623 and Rio. This is particularly interesting because a previous study with cDNAs from immature and maturing stem of sugarcane identified only 2.4% of the transcripts related to carbohydrate metabolism (Casu et al., 2003). Furthermore, because sorghum stems are fully elongated at the anthesis stage, tissue samples from maturing internodes were also more suitable in profiling changes in gene expression associated with carbohydrate metabolism. The implication is that screening of differentially expressed genes can greatly be enhanced by genetic variability and selection of tissue.

Function of Genes with Elevated Expression in Sweet Sorghum

The highest elevated transcript identified in our study encodes a Saposin-like type B domain. Increased expression has also been validated and tested in other sweet sorghum lines by RT-PCR. We also found a higher expression in Dale and Della compared to that in BTx623 (FIG. 2C). SAPOSINS are water soluble proteins that interact with the lysosomal membrane and are involved in the catabolism of glycosphingolipids in animals (Munford et al., 1995; Stokeley et al., 2007). Their role in sugar accumulation could be the removal of sugars from glycosphingolipids in the membrane, constituting an early step in carbohydrate partitioning. Additional transcripts that were increased in sweet sorghum included Hexokinase 8, Sorbitol Dehydrogenase and Carbohydrate Phosphorylase (starch phosphorylase). HEXOKINASE has a role not only in glycolysis but also as a glucose sensor that controls gene expression (Jang et al., 1997). SORBITOL DEHYDROGENASE is an enzyme involved in carbohydrate metabolism that converts the sugar alcohol form of glucose (sorbitol) into fructose (Zhou et al., 2006). Increased transcript levels of Carbohydrate Phosphorylase suggest that enhanced starch degradation in Rio may contribute to sugar accumulation. Another increased transcript encodes a NADP-malic enzyme suggesting that carbon fixation is enhanced in the stems of sweet versus grain sorghum. Indeed, the activity of enzymes involved in photosynthesis and the expression of their transcripts are modulated by sink strength. In sugarcane, the accumulation of sucrose in the maturing and mature internodes of the stem contribute greatly to sink strength (McCormick et al., 2006). Kinetic models have been proposed to explain sucrose accumulation in sugarcane (Rohwer and Botha, 2001; Uys et al., 2007). These models support the notion that sucrose accumulates in the vacuole against a concentration gradient. Indeed, we found that a transcript encoding a vacuolar ATP synthase catalytic subunit A had an increased expression in sweet sorghum, consistent with the role of this ATP synthase in the generation of an electrochemical gradient across the vacuolar membrane to propel the transport of sucrose.

The only cell wall-related transcript that was up regulated in sweet sorghum encodes a lysine motif (LysM) containing protein. The LysM domain is widespread in bacterial proteins that degrade cell walls but is also present in eukaryotes. They are assumed to have a general role in peptidoglycan binding (Bateman and Bycroft, 2000).

Mobilization of Sugars in the Stems of Sweet Sorghum

Interestingly, genes with reduced transcript levels outpaced those with increased levels by a 2:1 margin. Down regulated transcripts involved in the starch and sucrose metabolic pathway found in our study included Alpha-galactosidase, Beta-galactosidase, Sucrose Synthase 2 and Fructokinase 2. ALPHA and BETA-GALACTOSIDASE enzymes are O-glycosyl hydrolases that hydrolyse the glycosidic bond between two or more carbohydrates or between a carbohydrate and a non-carbohydrate moiety (Henrissat et al., 1996). SUCROSE SYNTHASE is involved in the reversible conversion of sucrose to UDP-glucose and fructose (Koch, 2004). UDP-glucose can then be used as a substrate for starch and cell wall synthesis. Fructose instead is converted into fructose-6-phosphate by FRUCTOKINASE and further metabolized through glycolysis (Pego and Smeekens, 2000). Our findings are in agreement with previous reports showing that the onset of sucrose accumulation in Rio was accompanied by a decrease in sucrose synthase activity in stem tissue (Lingle, 1987). Similarly, Tarpley et al. (1994) proposed that a decline in the levels of sucrose synthase may be necessary for sucrose accumulation at stem maturity in sorghum (Tarpley et al., 1994). Consistent with our findings, Xue et al. (2007) have recently reported the down-regulation in the expression of both Sucrose Synthase and Fructokinase genes in the stems of wheat genotypes with high water-soluble carbohydrates (Xue et al., 2008).

Reduced Expression of Cellulose and Lignocellulose-Related Genes in Sweet Sorghum Stems

Several transcripts involved in cell wall-related processes were identified as down regulated in sweet sorghum. These included cellulose synthase 1, 7, and 9 as well as cellulose synthase catalytic subunit 12 in cellulose synthesis. In the case of lignin biosynthesis we found transcripts such as phenylalanine and histidine ammonia-lyase, cinnamoyl CoA reductase, 4-coumarate coenzyme A ligase and caffeoyl-CoA O-methyltransferases. Interestingly, the expression of two transcripts encoding for xylanase inhibitors were also down regulated in sweet sorghum. Xylanase inhibitors proteins belong to the group of protein inhibitors of cell wall degrading enzymes (CWDEs). Xylan is the major hemicellulose polymer in cereals and is degraded by plant endoxylanases (Juge et al., 2006). This suggests that in sweet sorghum the degradation of hemicellulose is promoted by suppressing the expression of xylanases inhibitors.

In other cases, a decrease in the expression of cellulose synthase genes in wheat genotypes with high water-soluble carbohydrate content has also been observed (Xue et al., 2008). In addition, Casu et al (2007) have recently characterized the expression of several Cellulose synthase and Cellulose synthase-like genes in sugarcane stem and found that their expression is highly variable depending on internode maturity (Casu et al., 2007).

Reduced Higher-Order Components in Sweet Sorghum Stems

In addition to cellulose synthesis, the geometric deposition of cellulose fibrils generally perpendicular to the axis of cell elongation is a critical step in cell wall formation. There is evidence that the orientation of cellulose deposition is somehow assisted by microtubules (Somerville et al., 2004). An example of this is the fiber fragile mutant fra1 encoding a kinesin-like protein. In this mutant, cellulose deposition displayed an abnormal orientation (Burk and Ye, 2002). Consistent with these observations, the expression of two transcripts encoding tubulin alpha-2/alpha-4 chain and tubulin folding cofactor A, in conjunction with a transcript encoding a protein with kinesin motor domain were all down regulated in sweet sorghum.

Less clear, but also related to cell wall formation is Fasciclin. Interestingly, the most strongly down-regulated transcript in sweet sorghum encodes a protein with a Fasciclin domain. Fasciclin domains are found in animal arabinogalactan proteins that have a role in cell adhesion and communication (Kawamoto et al., 1998). These proteins are structural components that mediate the interaction between the plasma membrane and the cell wall. However, their specific role in plants is still unknown (Faik et al., 2006). A loss-of-function mutant in the Arabidopsis gene Fasciclin-like Arabinogalactan 4 (AtFLA4) displayed thinner cell walls and increased sensitivity to salinity (Yang et al., 2007).

Reduced Cross-Linking in Sweet Sorghum Stems

Other transcripts that were also down regulated encode a peroxidase and a laccase. It has been shown that peroxidases have an important role in cell wall modification (Passardi et al., 2004). By controlling the abundance of H₂O₂ in the cell wall, a necessary step for the cross linking of phenolic compounds, peroxidases act to inhibit cell elongation, and in conjunction with laccases, are assumed to be involved in monolingol unit oxidation, a reaction necessary for lignin assembly. Furthermore, it is known that peroxidase activity can be controlled by ascorbate. Indeed, the expression of a transcript encoding a protein similar to GDP-mannose 3, 5-epimerase was increased in sweet sorghum. This protein catalyzes the reversible conversion of GDP-mannose either into GDP-L-galactose or a novel intermediate, GDP-gulose, a step necessary for the biosynthesis of vitamin C in plants (Wolucka and Van Montagu, 2003). In addition, GDP-mannose is used to incorporate mannose residues into cell wall polymers (Lukowitz et al., 2001). For these reasons, it is considered that GDP-mannose 3,5 epimerase could modulate the carbon flux into the vitamin C pathway as well as the demand for GDP-mannose into the cell wall biosynthesis (Wolucka and Van Montagu, 2003). Indeed, it is known that the stem of high-sucrose-accumulating genotypes of sugarcane are high in moisture content and low in fiber whereas the stem of low-sucrose-accumulating genotypes are low in moisture content, thin and fibrous (Bull and Glasziou, 1963).

Compensation of Osmotic Shock in Sweet Sorghum Stems

Consistent with the idea that high concentration of sugars imposes osmotic stress to the cell, we found increased transcripts encoding heat shock proteins HSP70 and HSP90. Additionally, a transcript encoding a Poly ADP-ribose polymerase 2 (PARP 2) was significantly down regulated in sweet sorghum. This is in agreement with a recent report in which Arabidopsis and Brassica napus transgenic plants with reduced levels of PARP 2 displayed resistance to various abiotic stresses (Vanderauwera et al., 2007). Poly ADP-ribosylation involves the tagging of proteins with long-branched poly ADP-ribose polymers and is mediated by PARP enzymes (Schreiber et al., 2006). Poly ADP-ribosylation has important roles in the cellular response to genotoxic stress, influence DNA synthesis and repair, and is also involved in chromatin structure and transcription.

Mapping Genes Linked to Sugar Content and Cell Wall Metabolism in Sorghum and Rice

Although sugarcane has not been sequenced yet, we can use the sequenced genome of sorghum to construct a “transcriptome map” with the genes found in our study. Assuming that gene order has been largely conserved between these closely related species, the “transcriptome map” of sorghum serves as a valuable reference for sugarcane. We could not find any particular clustering of these genes but did observe that most of the genes are located towards the telomeres and only a few of them near the centromeres. We also could not find any of these genes in the telomeric region on the long arm of chromosome six.

Comparing this map with the rice genome demonstrated that out of 163 differentially expressed genes, 123 were in syntenic positions. With respect to the subset of genes involved in the accumulation of fermentable sugars and reduced lignocellulose, 21 genes were also found in syntenic regions whereas 10 genes appeared to be paralogous copies.

Outlook

Given the synteny of these genes between rice and sorghum, one can assume that they are allelic between different sorghum cultivars. Therefore, future genetic mapping experiments should provide a direct link of allelic variation and the sweet sorghum trait. Most likely, such allelic variations extend to the control regions of these genes because of their differential expression. Transgenic experiments can then be used to verify such functional aspects for biofuel properties. Moreover, gain of function experiments could be used to import desirable traits such as accumulation of fermentable sugars from sweet sorghum into maize. The generation of “sweet sorghum-like transgenic corn” will alleviate in part the increasing pressure of growing corn either for food or for biofuel since it would then be possible to use the grain for food and at the same time to extract fermentable sugars from the stem to use in ethanol production.

Genetic Transformation

One of ordinary skill in the art will appreciate the procedure utilized to perform the genetic transformation in accordance with practicing the present invention. In certain embodiments of the invention, genetic transformation in plants can be achieved by two methods: Agrobacterium-mediated transformation, particle bombardment and direct gene transfer into protoplasts. There are three basic requirements for the production of transgenic plants: 1) the availability of target tissues competent for plant regeneration, 2) a suitable method to introduce DNA into cells that can regenerate, and 3) a procedure to select and regenerate transformed plants with a reasonable frequency. While a decade ago it was difficult to transform grass species, it has now become a routine to adept existing methods to new grass species and even sorghum has been transformed recently (Gurel, Songul, Gurel, Ekrem, Kaur, Rajvinder, Wong, Joshua, Meng, Ling, Tan, Han-Qi Q, Lemaux, Peggy G. Efficient, reproducible Agrobacterium-mediated transformation of sorghum using heat treatment of immature embryos. Plant Cell Rep 2009 vol. 28 (3) pp. 429-44). Our experience has been with maize transformation (U.S. Pat. No. 6,849,779 B1), generally using the protocols published by the Center for Plant Transformation of Iowa State University (Frame, Bronwyn R, Shou, Huixia, Chikwamba, Rachel K, Zhang, Zhanyuan, Xiang, Chengbin, Fonger, Tina M, Pegg, Sue E, Li, Baochun, Nettleton, Dan S, Pei, Deqing, Wang, Kan. Agrobacterium tumefaciens-mediated transformation of maize embryos using a standard binary vector system. Plant Physiol. 2002 vol. 129 (1) pp. 13-22) (Wang, Kan, Frame, Bronwyn. Biolistic gun-mediated maize genetic transformation. Methods Mol Biol 2009 vol. 526 pp. 29-45) (and references cited therein).

### Demonstration of Gene Discovery Regulating High Sugar Content by Genetic Linkage Analysis

We have used next generation sequencing (ABI's SOLiD platform) to analyze small RNAs of stem tissue of Btx, Rio as well as of two pools of F2 plants, which exhibit high and low Brix degree (sugar content), respectively. We constructed small RNA libraries and sequenced the barcoded libraries. We then mapped the obtained sequences to the Btx623 genomic sequence and compared it to known miRNAs. For the miRNA172 we could show that the relative expression level of miRNA172a and miRNA172c is twice as high in Btx623 and low Brix F2 plants as compared to Rio and high Brix F2 plants, respectively. It also correlates with flowering time: high Brix degree is correlated with late flowering (resembling Btx parent phenotype) and low Brix is correlated with early flowering (resembling Rio parent phenotype). Remarkably, miRNA172a and miRNA172c are extremely abundant as they make up 0.7-2.6% of all small RNAs mapped to the sorghum genome.

We found that the expression level of two micro-RNA genes termed microRNAs 172a and c (miR172a and miR172c) co-segregate with sugar content in F2 plants. Particularly, we found that the expression level of miR172a and miR172c in Btx623 is twice as high to that in Rio. When the expression of these two microRNA genes was analyzed in F2 plants displaying low Brix and early flowering (resembling the Btx623 parent phenotype) and in F2 plants with high Brix and late flowering (resembling the Rio parent) we found that miR172a and miR172c expression level is twice as high in the low Brix and early flowering F2s compared to that in the high Brix and late flowering F2 plants. This means that the expression level difference in miR172a and miR172c between BTx623 and Rio is inherited in the F2 generation.

Previous work done with the model plant Arabidopsis thaliana demonstrated the role of mir172 in flowering time: over-expression of miR172 promotes early flowering. Interestingly, mir172 downregulates a subfamily of APETALA2 transcription factors (Aukerman, Milo J, Sakai, Hajime. Regulation of flowering time and floral organ identity by a MicroRNA and its APETALA2-like target genes. Plant Cell 2003 vol. 15 (11) pp. 2730-41). However, there is no report on the function of miR172 genes in sorghum and their possible link to influence sugar accumulation. Certainly, our finding is the first case demonstrating that.

This finding means that miR172a and miR172c (and the target genes they regulate), could be used to manipulate the flowering time, sugar content and biomass of sorghum to produce plants fully adapted to different geographic regions in where biofuel production may be required. One can envision increasing the expression of sorghum microRNA in sweet sorghum cultivars by standard genetic engineering techniques with the goal to increase stem sugar to higher levels of Brix degrees than achieved by conventional breeding.

### DETAILED DESCRIPTION OF PREFERRED EMBODIMENTS

### EXAMPLE 1

### Gene Identification

Plant Materials and Growth Conditions

Seeds from both grain and sweet sorghum (Sorghum bicolor (L.) Moench) were sown in pro-mix soil (Premiere Horticulture Inc., USA) and grown in our greenhouse with a day length of 15 hrs light: 9 hrs dark at constant temperature of 23° C. The genotype representing grain sorghum in our study was BTx623 whereas the genotypes representing sweet sorghum were Dale, Della, M81-E, Rio, Simon and Top76-6. The seeds from sweet sorghum were kindly provided by Dr. William L. Rooney of Texas A&M, College Station, Tex.

Measurement of “Brix Degree” from Sorghum Stem's Juice

The juice from internodes of the main stem in both grain and sweet sorghum was harvested at the time of anthesis. A section of approximately 6 cm long was dissected from the middle of each internode and 300 μl of juice was extracted by pressing each internode with a garlic squeezer. The concentration of total soluble sugars in the juice was measured with a pocket refractometer (Atago Inc., Japan).

Isolation of Total RNA from Stem Tissue

Both grain sorghum BTx623 and sweet sorghum Rio were grown until anthesis and total RNA from internode 8 for each genotype (internodes were numbered from the base towards the apex of the stem) was extracted using the RNeasy Plant Mini Kit (QIAGEN Inc., USA).

GeneChip Sugarcane Genome Array Hybridization

Sorghum RNA from internode 8 was hybridized to the Affymetrix GeneChip Sugarcane Genome Array (Affymetrix Inc., USA). Probe set information can be found at NetAffx Analysis Center's web page (www.affymetrix.com/analysis/index.affx). The One-Cycle Eukaryotic Target Labeling Assay protocol was used. The labeling, hybridization and data collection were done at the Transcription Profiling Facility, Cancer Institute of New Jersey (CINJ), Department of Pediatrics, Robert Wood Johnson Medical School (RWJMS).

Data Analysis

Probe sets that were absent in all chips were eliminated. About 5900 out of the original 8300 probe sets passed this test. Next, a t-test was applied to BTx623 and Rio groups (three replicates for each) with an alpha value of 0.001 and the Benjamini-Hochberg multiple-testing correction was applied. From the probe sets that passed the criteria, only those with a fold change of at least 2 were considered.

Validation of Microarray Data Through Semi-Quantitative RT-PCR

cDNA synthesis was performed from 500 ng of total RNA using the SuperScript III First-Strand Synthesis System for RT-PCR (Invitrogen). Oligo (dT) was used as primer for cDNA synthesis. Then 1 μl of cDNA was used for gene amplification. The PCR condition used was: 94° C. 2 minutes; 94° C. 30 seconds; 55° C. 30 seconds; 72° C. 30 seconds; 72° C. 5 minutes. The primers sequences for each gene as well as the PCR cycle used are listed in Supplemental table 3.

Physical Location of Differentially Expressed Transcripts in the Sorghum Genome

The sugarcane probe sets that were up and down regulated in Sorghum, respectively, were mapped to the genome by using GenomeThreader (Gremme et al., 2005). Spliced alignments were only considered if 75% (score>0.75) or more bases could be aligned between the genomic sequence and a probe set. If a probe could be mapped to the genome and if it also overlapped with a sorghum gene, we assigned the annotation of the sorghum gene to the probe.

The disclosures of each reference provided herein are hereby incorporated by reference in their entireties.

TABLE 1
List of “trait-specific” genes that are syntenic with rice.

| Gene¹ | Rice | Sorghum | Expression² |
|---|---|---|---|
| Starch and sucrose metabolism | | | |
| Hexokinase 8 | Os01g0190400 | Sb03g003190.1* | 2.3 |
| Hexokinase 8 | Os05g0187100 | Sb09g005840.1 | |
| Carbohydrate phosphorylase | Os01g0851700 | Sb03g040060.1* | 1.2 |
| Sucrose synthase 2\hat{ } | Os03g0401300 | Sb01g033060.1* | −1.3 |
| Sucrose synthase 2 | Os07g0616800 | | |
| Fructokinase-2 | Os08g0113100 | Sb07g001320.1* | −1.7 |
| Sorbitol dehydrogenase | Os08g0545200 | Sb07g025220.1* | 1.6 |
| Sugar binding | | | |
| D-mannose binding lectin | Os06g0165200 | Sb10g022730.1* | 2 |
| CO2 assimilation | | | |
| NADP dependent malic enzyme\hat{ } | Os01g0723400 | Sb03g033250.1* | 2 |
| Cell wall related | | | |
| LysM domain protein/cell wal | Os03g0110600 | Sb01g049890.1* | 1.2 |
| catabolism | | | |
| Cellulose synthase-7\hat{ } | Os03g0837100 | Sb01g002050.1* | −1 |
| Cellulose synthase-1\hat{ } | Os05g0176100 | Sb09g005280.1* | −1.1 |
| Cellulose synthase-9\hat{ } | Os07g0208500 | Sb02g006290.1* | −1.1 |
| Cellulose synthase-9 | Os03g0808100 | Sb01g004210.1 | |
| Cellulose synthase catalytic subunit 12 | Os09g0422500 | Sb02g025020.1* | −4.7 |
| Alpha-galactosidase precursor | Os10g0493600 | Sb01g018400.1* | −1.8 |
| Beta-galactosidase 3 precursor | Os01g0875500 | Sb03g041450.1* | −2.4 |
| Beta-galactosidase 3 precursor | Os05g0428100 | Sb03g041450.1 | |
| Cinnamoyl CoA reductase | Os08g0441500 | Sb07g021680.1* | −2.9 |
| Cinnamoyl CoA reductase | Os09g0419200 | Sb10g005700.1 | |
| Laccase | Os01g0842400 | Sb03g039520.1 | −3.5 |
| 4-Coumarate coenzyme A ligase | Os02g0177600 | Sb04g005210.1* | −3.7 |
| 4-Coumarate coenzyme A ligase | Os06g0656500 | Sb10g026130.1 | |
| Fasciclin domain | Os03g0788600 | Sb01g005770.1* | −1.75 |
| Fasciclin domain | Os07g0160600 | Sb02g003410.1 | |
| Fasciclin-like protein FLA15 | Os05g0563600 | Sb09g028490.1* | −6.5 |
| Caffeoyl-CoA O-methyltransferase 2\hat{ } | Os06g0165800 | Sb10g004540.1* | −2.15 |
| Caffeoyl-CoA O-methyltransferase\hat{ } | Os08g0498100 | Sb07g028530.1* | −5.3 |
| Caffeoyl-CoA O-methyltransferase | Os09g0481400 | Sb02g027930.1 | |

¹Paralogs in italics
²Mean Log2 Ratio of sweet versus grain sorghum
*Sorghum gene to which a sugarcane probe set was mapped.
\hat{ }Sorghum genes that correspond to sugarcane probe set IDs previously reported by Casu et al. 2007

TABLE 2
List of “trait-specific” genes that are not syntenic with rice.

| Gene | Sorghum | Expression^{a} |
|---|---|---|
| Cell wall related | | |
| Alcohol dehydrogenase\hat{ } | Sb10g006290 | 1 |
| Cinnamyl alcohol dehydrogenase | Sb04g011550 | −1.5 |
| Dolichyl-diphospho-oligosaccharide | Sb02g006330 | −1.4 |
| Xyloglucan endo-transglycosylase/ | Sb06g015880 | −1.1 |
| hydrolase | | |
| Putative Xylanase inhibitor | Sb05g027350 | −1.5 |
| Putative Xylanase inhibitor | Sb02g004660 | −1.5 |
| Glycoside hydrolase family 1\hat{ } | Sb02g029640 | −1.1 |
| Phenylalanine and histidine ammonia- | Sb04g026520 | −2 |
| lyase | | |
| Peroxidase | Sb02g037840 | −1.5 |
| Similar to Saposin type B protein | Sb09g013990 | 5.7 |

\hat{ }Sorghum genes that correspond to sugarcane probe set IDs previously reported by Casu et al. 2007
^{a}Mean Log2 Ratio of sweet versus grain sorghum

SUPPLEMENTAL TABLE 1
List of differentially expressed genes between grain and sweet sorghum that have an orthologous copy in a syntenic position in rice.

| Sugarcane Probe Set ID | Expression | Sb Gene ID | OsRAP2 Gene ID | Sb Function | PFAM | Description | GO-Term |
|---|---|---|---|---|---|---|---|
| Up-regulated | | | | | | | |
| Starch and sucrose metabolism | | | | | | | |
| SOF.4315.1.S1_AT | 2.3 | Sb03g003190.1 | Os01g0190400 | similar to Hexokinase-8 | PF03727 | Hexokinase | GO:0006096 |
| | | | | | PF00349 | Hexokinase | GO:0006096 |
| SOF.90.1.S1_AT | 1.2 | Sb03g040060.1 | Os01g0851700 | similar to Phosphorylase | PF00343 | Carbohydrate | GO:0005975 |
| | | | | | | phosphorylase | |
| Sugar binding | | | | | | | |
| SOF.1513.1.A1_AT | 2 | Sb10g022730.1 | Os06g0165200 | similar to Putative | PF01453 | D-mannose | GO:0005529 |
| | | | | uncharacterized protein | | binding lectin | |
| | | | | | PF00024 | PAN domain | N/A |
| Cell wall catabolism | | | | | | | |
| SOF.3731.1.A1_AT | 1.2 | Sb01g049890.1 | Os03g0110600 | similar to LysM domain | PF01476 | LysM domain | GO:0016998 |
| | | | | containing protein, | | | |
| | | | | expressed | | | |
| Transcription factor | | | | | | | |
| SOFAFFX.287.1.S1_AT | 2.2 | Sb10g007380.1 | Os06g0217300 | similar to M21 protein | PF01486 | K-box region | GO:0005634 |
| | | | | | PF00319 | SRF-type | GO:0005634 |
| | | | | | | transcription | |
| | | | | | | factor (DNA- | |
| | | | | | | binding and | |
| | | | | | | dimerization | |
| | | | | | | domain) | |
| SOF.2682.1.S1_AT | 2 | Sb01g013710.1 | Os12g0612700 | similar to Class III HD-Zip | PF00046 | Homeobox | GO:0005634 |
| | | | | protein 4, putative, | | domain | |
| | | | | expressed | | | |
| | | | | | PF01852 | START domain | N/A |
| SOFAFFX.142.1.S1_AT | 1.6 | Sb04g005620.1 | Os06g0646600 | similar to KNOX family | PF03791 | KNOX2 domain | GO:0005634 |
| | | | | class 2 homeodomain | | | |
| | | | | protein | | | |
| | | | | | PF03789 | ELK domain | GO:0005634 |
| SOF.3290.1.S1_AT | 1.1 | Sb08g016240.1 | Os12g0507300 | similar to Os12g0507300 | PF03106 | WRKY DNA - | GO:0005634 |
| | | | | protein | | binding domain | |
| Zinc-ion binding | | | | | | | |
| SOFAFFX.1438.1.A1_S_AT | 2 | Sb09g006050.1 | Os01g0192000 | similar to Putative | PF00642 | Zinc finger C- | GO:0008270 |
| | | | | uncharacterized protein | | x8-C-x5-C-x3- | |
| | | | | | | H type (and | |
| | | | | | | similar) | |
| SOF.603.1.A1_A_AT | 1.6 | Sb07g025220.1 | Os08g0545200 | similar to Sorbitol | PF08240 | N/A | N/A |
| | | | | dehydrogenase | | | |
| | | | | | PF00107 | Zinc-binding | N/A |
| | | | | | | dehydrogenase | |
| SOF.4452.1.A1_AT | 1.3 | Sb04g021610.1 | Os02g0530300 | similar to Zinc finger A20 | PF01754 | A20-like zinc | GO:0008270 |
| | | | | and AN1 domain-containing | | finger | |
| | | | | stress-associated protein 5 | | | |
| | | | | | PF01428 | AN1-like Zinc | GO:0008270 |
| | | | | | | finger | |
| SOF.1992.2.S1_AT | 1.2 | Sb02g039390.1 | Os07g0618600 | similar to Os07g0618600 | PF01363 | FYVE zinc | GO:0008270 |
| | | | | protein | | finger | |
| | | | | | PF00023 | Ankyrin repeat | N/A |
| Oxidoreductase activity | | | | | | | |
| SOF.1594.1.S1_AT\hat{ } | 2 | Sb03g033250.1 | Os01g0723400 | similar to NADP dependent | PF00390 | Malic enzyme, | GO:0016616 |
| | | | | malic enzyme | | N-terminal | |
| | | | | | | domain | |
| | | | | | PF03949 | Malic enzyme, | GO:0051287 |
| | | | | | | NAD binding | |
| | | | | | | domain | |
| SOF.398.1.A1_AT | 1.4 | Sb02g043370.1 | Os07g0685800 | similar to Carbonyl | PF00106 | short chain | GO:0008152 |
| | | | | reductase-like protein | | dehydrogenase | |
| Carboxy-lyase activity | | | | | | | |
| SOF.3466.1.A1_AT | 1.6 | Sb07g022670.2 | Os08g0465800 | similar to GAD1 | PF00282 | Pyridoxal- | GO:0019752 |
| | | | | | | dependent | |
| | | | | | | decarboxylase | |
| | | | | | | conserved | |
| | | | | | | domain | |
| Translation initiation | | | | | | | |
| SOF.3301.1.S1_AT | 1.4 | Sb03g047210.1 | Os01g0970400 | similar to Eukaryotic | PF01652 | Eukaryotic | GO:0005737 |
| | | | | translation initiation factor | | initiation factor | |
| | | | | 4E-1 | | 4E | |
| Protein binding | | | | | | | |
| SOF.2770.2.S1_X_AT | 1.4 | Sb03g041770.1 | Os01g0881900 | similar to Putative | PF00646 | F-box domain | N/A |
| | | | | uncharacterized protein | | | |
| | | | | | PF00560 | Leucine Rich | GO:0005515 |
| | | | | | | Repeat | |
| Protein catabolism | | | | | | | |
| SOFAFFX.1586.1.S1_AT | 1.3 | Sb03g025180.1 | Os01g0550100 | similar to Ubiquitin | PF00240 | Ubiquitin | GO:0006464 |
| | | | | carboxyl-terminal hydrolase | | family | |
| | | | | | PF00443 | Ubiquitin | GO:0006511 |
| | | | | | | carboxyl- | |
| | | | | | | terminal | |
| | | | | | | hydrolase | |
| SOF.1683.1.S1_AT | 1.2 | Sb01g043060.1 | Os03g0212700 | similar to Mitochondrial | PF00675 | Insulinase | GO:0006508 |
| | | | | processing peptidase beta | | (Peptidase | |
| | | | | subunit | | family M16) | |
| | | | | | PF05193 | Peptidase M16 | GO:0006508 |
| | | | | | | inactive domain | |
| Electron transport | | | | | | | |
| SOFAFFX.1192.1.S1_AT | 1.3 | Sb03g027710.1 | Os01g0612200 | similar to Cytochrome c | PF01215 | Cytochrome c | GO:0005740 |
| | | | | oxidase subunit Vb | | oxidase subunit | |
| | | | | | | Vb | |
| SOF.2692.1.S1_AT | 1 | Sb08g002250.1 | Os12g0119000 | similar to Cytochrome P45051 | PF00067 | Cytochrome | GO:0006118 |
| | | | | | | P450 | |
| Membrane associated protein | | | | | | | |
| SOF.4998.1.S1_AT | 1.3 | Sb10g002420.1 | Os06g0136000 | similar to Hypersensitive- | PF01145 | SPFH domain/ | N/A |
| | | | | induced reaction protein 4 | | Band 7 family | |
| Alternative splicing | | | | | | | |
| SOF.3633.1.S1_AT | 1.3 | Sb01g046550.3 | Os03g0158500 | similar to YT521-B-like | PF04146 | YT521-B-like | N/A |
| | | | | family protein, expressed | | family | |
| Chaperonin activity | | | | | | | |
| SOF.3437.1.S1_AT | 1.3 | Sb09g022580.1 | Os01g0840100 | similar to Heat shock | PF00012 | Hsp70 protein | N/A |
| | | | | cognate 70 kDa protein | | | |
| Kinase activity | | | | | | | |
| SOFAFFX.494.1.S1_S_AT | 1.2 | Sb10g001310.1 | Os06g0116100 | similar to Putative GAMYB- | PF00069 | Protein kinase | GO:0006468 |
| | | | | binding protein | | domain | |
| | | | | | PF07714 | Protein tyrosine | GO:0006468 |
| | | | | | | kinase | |
| Transferase activity | | | | | | | |
| SOF.1326.1.S1_A_AT | 1.2 | Sb02g000780.1 | Os07g0108300 | similar to Alanine | PF00155 | Aminotransferase | GO:0009058 |
| | | | | aminotransferase | | class I and II | |
| Proton transport | | | | | | | |
| SOF.3139.1.S1_AT\hat{ } | 1.1 | Sb10g026440.1 | Os02g0175400 | similar to Vacuolar ATP | PF02874 | ATP synthase | GO:0016469 |
| | | | | synthase catalytic subunit A | | alpha/beta | |
| | | | | | | family, beta- | |
| | | | | | | barrel domain | |
| | | | | | PF00006 | ATP synthase | GO:0016469 |
| | | | | | | alpha/beta | |
| | | | | | | family, | |
| | | | | | | nucleotide- | |
| | | | | | | binding domain | |
| SOFAFFX.1600.2.A1_AT | 1 | Sb09g027790.1 | Os01g0685800 | similar to ATP synthase | PF02874 | ATP synthase | GO:0016469 |
| | | | | subunit beta, mitochondrial | | alpha/beta | |
| | | | | precursor | | family, beta- | |
| | | | | | | barrel domain | |
| | | | | | PF00006 | ATP synthase | GO:0016469 |
| | | | | | | alpha/beta | |
| | | | | | | family, | |
| | | | | | | nucleotide- | |
| | | | | | | binding domain | |
| Arginine biosynthesis | | | | | | | |
| SOFAFFX.1412.1.A1_S_AT | 1 | Sb08g008320.1 | Os12g0235800 | similar to Argininosuccinate | PF00764 | Arginosuccinate | GO:0006526 |
| | | | | synthase | | synthase | |
| Metabolic process | | | | | | | |
| SOF.4917.1.S1_AT | 1 | Sb03g004390.1 | Os05g0171000 | similar to Phospholipase D | PF00168 | C2 domain | N/A |
| | | | | alpha 1 | | | |
| | | | | | PF00614 | Phospholipase | GO:0008152 |
| | | | | | | D Active site | |
| | | | | | | motif | |
| DNA methylamino | | | | | | | |
| SOF.3784.1.A1_AT | 1 | Sb02g004680.1 | Os07g0182900 | similar to Cytosine-specific | PF01426 | BAH domain | GO:0003677 |
| | | | | methyltransferase | | | |
| Response to stress | | | | | | | |
| SOF.2151.1.S1_AT | 1 | Sb09g004470.1 | Os05g0157200 | similar to Putative | PF00582 | Universal stress | GO:0006950 |
| | | | | uncharacterized protein | | protein family | |
| | | | | P0676G05.12 | | | |
| Vitamin C Synthesis | | | | | | | |
| SOFAFFX.630.1.S1_AT | 1.1 | Sb05g022890.1 | Os11g0591100 | similar to GDP-mannose | Pfam:N/A | Func:N/A | GO:N/A |
| | | | | 3,5-epimerase 1 | | | |
| Unknown function | | | | | | | |
| SOF.1282.2.S1_A_AT | 1.4 | Sb02g023980.1 | Os09g0386600 | similar to Putative | Pfam:N/A | Func:N/A | GO:N/A |
| | | | | uncharacterized protein | | | |
| SOF.2601.1.S1_AT | 1.3 | Sb08g016302.1 | Os12g0508200 | similar to Expressed protein | Pfam:N/A | Func:N/A | GO:N/A |
| SOF.3798.1.S1_AT | 1.2 | Sb02g025720.1 | Os09g0439200 | similar to Putative | PF06200 | ZIM motif | N/A |
| | | | | uncharacterized protein | | | |
| SOF.366.1.S1_S_AT\|SOF.366.2.S1_S_AT | 1.1\|1.3 | Sb01g002220.1 | Os03g0835150 | similar to Expressed protein | Pfam:N/A | Func:N/A | GO:N/A |
| SOF.2346.1.S1_AT | 1.1 | Sb03g028860.1 | Os01g0633200 | similar to X1 | PF03469 | XH domain | N/A |
| | | | | | PF03468 | XS domain | N/A |
| SOF.32.1.S1_AT | 1 | Sb01g045110.1 | Os03g0182400 | similar to SacIy domain | PF02383 | SacI homology | N/A |
| | | | | containing protein, | | domain | |
| | | | | expressed | | | |
| Down-regulated | | | | | | | |
| Sucrose metabolism | | | | | | | |
| SOF.4165.1.S1_S_AT\hat{ } | −1.3 | Sb01g033060.1 | Os03g0401300 | similar to Sucrose synthase 2 | PF00862 | Sucrose | GO:0005985 |
| | | | | | | synthase | |
| | | | | | PF00534 | Glycosyl | GO:0009058 |
| | | | | | | transferases | |
| | | | | | | group 1 | |
| SOF.3644.2.S1_A_AT | −1.7 | Sb07g001320.1 | Os08g0113100 | similar to Fructokinase-2 | PF00294 | pfkB family | N/A |
| | | | | | | carbohydrate | |
| | | | | | | kinase | |
| Cell wall related | | | | | | | |
| SOF.1587.3.A1_A_AT\hat{ } | −1 | Sb01g002050.1 | Os03g0837100 | similar to Cellulose | PF03552 | Cellulose | GO:0016020 |
| | | | | synthase-7 | | synthase | |
| SOF.5033.1.S1_AT\hat{ } | −1.1 | Sb09g005280.1 | Os05g0176100 | similar to Cellulose | PF03552 | Cellulose | GO:0016020 |
| | | | | synthase-1 | | synthase | |
| SOF.4824.2.S1_A_AT\|SOFAFFX.1961.1.S1_S_AT\hat{ } | −1\|−1.2 | Sb02g006290.1 | Os03g0808100 | similar to Cellulose | PF03552 | Cellulose | GO:0016020 |
| | | | | synthase-9 | | synthase | |
| SOF.2699.2.S1_A_AT | −4.7 | Sb02g025020.1 | Os09g0422500 | similar to Cellulose synthase | PF03552 | Cellulose | GO:0016020 |
| | | | | catalytic subunit 12 | | synthase | |
| SOF.3244.1.S1_A_AT | −1.8 | Sb01g018400.1 | Os10g0493600 | similar to Alpha- | PF02065 | Melibiase | GO:0005975 |
| | | | | galactosidase precursor | | | |
| SOF.4934.1.S1_AT | −2.4 | Sb03g041450.1 | Os05g0428100 | similar to Beta-galactosidase | PF02140 | Galactose | GO:0005529 |
| | | | | 3 precursor | | binding lectin | |
| | | | | | | domain | |
| | | | | | PF02837 | Glycosyl | GO:0005975 |
| | | | | | | hydrolases | |
| | | | | | | family 2, sugar | |
| | | | | | | binding domain | |
| SOF.3629.1.S1_AT | −2.9 | Sb07g021680.1 | Os09g0419200 | similar to Cinnamoyl CoA | PF05368 | NmrA-like | GO:0006808 |
| | | | | reductase | | family | |
| | | | | | PF01073 | 3-beta | GO:0006694 |
| | | | | | | hydroxysteroid | |
| | | | | | | dehydrogenase/ | |
| | | | | | | isomerase family | |
| SOFAFFX.292.1.S1_AT\|SOF.5198.2.S1_A_AT\hat{ } | −1.4\|−2.9 | Sb10g004540.1 | Os06g0165800 | similar to Caffeoyl-CoA O- | PF01596 | O- | GO:0008171 |
| | | | | methyltransferase 2 | | methyltransferase | |
| SOF.1122.2.S1_A_AT\hat{ } | −5.3 | Sb07g028530.1 | Os09g0481400 | similar to Caffeoyl-CoA O- | PF01596 | O- | GO:0008171 |
| | | | | methyltransferase | | methyltransferase | |
| SOF.1021.1.A1_AT | −3.5 | Sb03g039520.1 | Os01g0842400 | similar to Putative laccase | PF00394 | Multicopper | GO:0016491 |
| | | | | | | oxidase | |
| | | | | | PF07731 | Multicopper | GO:0016491 |
| | | | | | | oxidase | |
| SOF.4734.1.S1_AT | −3.7 | Sb04g005210.1 | Os02g0177600 | similar to 4-coumarate | PF00501 | AMP-binding | GO:0008152 |
| | | | | coenzyme A ligase | | enzyme | |
| Cell adhesion | | | | | | | |
| SOFAFFX.1406.1.S1_AT\|SOF.4464.1.A1_AT | −1.9\|−1.6 | Sb01g005770.1 | Os03g0788600 | similar to Expressed protein | PF02469 | Fasciclin | GO:0007155 |
| | | | | | | domain | |
| SOF.3590.1.S1_AT | −6.5 | Sb09g028490.1 | Os05g0563600 | similar to Fasciclin-like | PF02469 | Fasciclin | GO:0007155 |
| | | | | protein FLA15 | | domain | |
| Carbohydrate metabolic process | | | | | | | |
| SOF.4949.1.S1_AT | −1.3 | Sb03g045390.1 | Os01g0939600 | similar to Os01g0939600 | PF01210 | NAD-dependent | GO:0005737 |
| | | | | protein | | glycerol-3- | |
| | | | | | | phosphate | |
| | | | | | | dehydrogenase | |
| | | | | | | N-terminus | |
| | | | | | PF07479 | NAD-dependent | GO:0005975 |
| | | | | | | glycerol-3- | |
| | | | | | | phosphate | |
| | | | | | | dehydrogenase | |
| | | | | | | C-terminus | |
| Water transport | | | | | | | |
| SOF.863.1.S1_S_AT | −1 | Sb10g008090.1 | Os06g0228200 | similar to Aquaporin NIP2-3 | PF00230 | Major intrinsic | GO:0016020 |
| | | | | | | protein | |
| Protein binding | | | | | | | |
| SOF.5088.1.S1_AT | −1 | Sb04g027910.2 | Os02g0748300 | similar to Kelch repeat- | PF07646 | Kelch motif | N/A |
| | | | | containing F-box-like | | | |
| | | | | | PF00646 | F-box domain | N/A |
| SOF.4911.1.S1_AT | −1.5 | Sb01g045010.1 | Os03g0183800 | similar to Leucine-rich | PF00560 | Leucine Rich | GO:0005515 |
| | | | | repeat transmembrane | | Repeat | |
| | | | | protein kinase 2 | | | |
| Mitochondrial envelop/electron transport | | | | | | | |
| SOF.4557.1.S1_AT | −1 | Sb03g037870.1 | Os01g0814900 | similar to Cytochrome b5 | PF00970 | Oxidoreductase | GO:0006118 |
| | | | | reductase | | FAD-binding | |
| | | | | | | domain | |
| | | | | | PF00175 | Oxidoreductase | GO:0006118 |
| | | | | | | NAD-binding | |
| | | | | | | domain | |
| DNA binding/transcription factor | | | | | | | |
| SOF.3143.2.S1_A_AT | −1 | Sb03g043690.1 | Os01g0915600 | similar to Putative | PF00010 | Helix-loop- | GO:0005634 |
| | | | | uncharacterized protein | | helix DNA- | |
| | | | | | | binding domain | |
| SOF.2024.1.S1_AT | −1.4 | Sb07g020090.1 | Os08g0408500 | similar to DRE binding | PF00847 | AP2 domain | GO:0005634 |
| | | | | factor 1 | | | |
| SOFAFFX.1576.1.S1_AT | −3.2 | Sb03g030750.1 | Os01g0672100 | similar to No apical | PF02365 | No apical | GO:0045449 |
| | | | | meristem (NAM) protein- | | meristem | |
| | | | | like | | (NAM) protein | |
| Kinase activity | | | | | | | |
| SOF.1818.1.S1_AT | −1 | Sb02g037070.1 | Os07g0572800 | similar to Mitogen activated | PF00069 | Protein kinase | GO:0006468 |
| | | | | protein kinase kinase | | domain | |
| Transferase activity | | | | | | | |
| SOF.1190.1.S1_AT | −1 | Sb07g005930.1 | Os08g0205900 | similar to Putative | PF00202 | Aminotransferase | GO:0030170 |
| | | | | uncharacterized protein | | class-III | |
| SOF.701.1.S1_AT | −1.3 | Sb03g003390.1 | Os01g0185300 | similar to Putative acyl | PF02458 | Transferase | N/A |
| | | | | transferase 3 | | family | |
| SOF.521.2.S1_AT | −1.1 | Sb10g002230.1 | Os06g0133900 | similar to 3- | PF00275 | EPSP synthase | GO:0016765 |
| | | | | phosphoshikimate 1- | | (3- | |
| | | | | carboxyvinyltransferase | | phosphoshikimate 1- | |
| | | | | | | carboxyvinyltransferase) | |
| SOFAFFX.409.1.S1_AT | −3.8 | Sb06g021640.1 | Os04g0500700 | similar to | PF02458 | Transferase | N/A |
| | | | | OSJNBa0029H02.19 protein | | family | |
| Nucleoside Transport | | | | | | | |
| SOF.3699.1.A1_AT | −1.4 | Sb07g005850.1 | Os08g0205200 | similar to Equilibrative | PF01733 | Nucleoside | GO:0016020 |
| | | | | nucleoside transporter 1 | | transporter | |
| Cation transport | | | | | | | |
| SOF.1478.1.A1_AT | −1.4 | Sb02g005440.1 | Os07g0191200 | similar to Cation- | PF00690 | Cation | GO:0016020 |
| | | | | transporting ATPase | | transporter/ATPase, | |
| | | | | | | N-terminus | |
| Transporter activity | | | | | | | |
| SOF.2138.1.S1_AT | −1.9 | Sb04g028300.1 | Os02g0741800 | similar to Root uracil | PF00860 | Permease | GO:0016020 |
| | | | | permease 1 | | family | |
| Zinc-ion binding | | | | | | | |
| SOF.808.1.S1_AT | −1.1 | Sb09g000820.1 | Os05g0106000 | similar to Putative | PF00096 | Zinc finger, | GO:0005622 |
| | | | | uncharacterized protein | | C2H2 type | |
| Metabolic process | | | | | | | |
| SOF.4186.2.S1_AT | −1.1 | Sb06g015180.1 | Os04g0404800 | similar to H0502B11.5 | PF00501 | AMP-binding | GO:0008152 |
| | | | | protein | | enzyme | |
| Cysteine protease inhibitor activity | | | | | | | |
| SOF.117.1.S1_AT | −1.1 | Sb09g024230.1 | Os05g0494200 | similar to Cystatin | PF00031 | Cystatin domain | GO:0004869 |
| Hydrolase activity | | | | | | | |
| SOF.4601.1.S1_AT | −1.2 | Sb01g041550.1 | Os03g0238600 | similar to Purple acid | PF00149 | Calcineurin-like | GO:0016787 |
| | | | | phosphatase 1, putative, | | phosphoesterase | |
| | | | | expressed | | | |
| Kreb's cycle/transferase activity | | | | | | | |
| SOF.2225.1.S1_AT | −2.2 | Sb04g006440.1 | Os02g0194100 | similar to Citrate synthase | PF00285 | Citrate synthase | GO:0046912 |
| Electron transport | | | | | | | |
| SOF.1998.1.A1_AT | −1.3 | Sb02g036870.1 | Os07g0570550 | similar to Chromosome chr5 | PF02298 | Plastocyanin- | GO:0006118 |
| | | | | scaffold_2, whole genome | | like domain | |
| | | | | shotgun sequence | | | |
| Protein translation | | | | | | | |
| SOF.4846.1.S1_AT\|SOF.4846.2.S1_A_AT | −1.5\|−1.2 | Sb04g007760.1 | Os02g0220600 | similar to Elongation factor | PF00647 | Elongation | GO:0005853 |
| | | | | 1-gamma 1 | | factor 1 gamma, | |
| | | | | | | conserved | |
| | | | | | | domain | |
| | | | | | PF00043 | Glutathione S- | N/A |
| | | | | | | transferase, C- | |
| | | | | | | terminal domain | |
| SOF.3827.1.S1_S_AT | −1.7 | Sb07g002560.1 | Os08g0130500 | similar to 60S acidic | PF00428 | 60s Acidic | GO:0005840 |
| | | | | ribosomal protein P0 | | ribosomal | |
| | | | | | | protein | |
| | | | | | PF00466 | Ribosomal | GO:0005622 |
| | | | | | | protein L10 | |
| SOF.177.2.A1_AT | −1.8 | Sb03g007840.1 | Os01g0120800 | similar to Eukaryotic | PF01399 | PCI domain | N/A |
| | | | | translation initiation factor 3 | | | |
| | | | | subunit 10 | | | |
| SOFAFFX.1035.1.S1_S_AT | −2 | Sb09g023950.1 | Os01g0812800 | similar to 60S ribosomal | PF01248 | Ribosomal | N/A |
| | | | | protein L30 | | protein | |
| | | | | | | L7Ae/L30e/S12e/ | |
| | | | | | | Gadd45 | |
| | | | | | | family | |
| SOF.1902.1.S1_S_AT | −2.6 | Sb05g001680.1 | Os12g0124200 | similar to 40S ribosomal | PF00380 | Ribosomal | GO:0005840 |
| | | | | proteinS 16 | | protein S9/S16 | |
| Trypsin-alpha amylase inhibitor | | | | | | | |
| SOF.3279.1.S1_AT | −1.4 | Sb08g002660.1 | Os12g0115300 | similar to Non-specific lipid- | PF00234 | Protease | N/A |
| | | | | transfer protein | | inhibitor/seed | |
| | | | | | | storage/LTP | |
| | | | | | | family | |
| Methionine metabolism | | | | | | | |
| SOF.3126.1.S1_AT | −1.4 | Sb01g003700.1 | Os03g0815200 | similar to | PF02219 | Methylenetetrahydrofolate | GO:0006555 |
| | | | | Methylenetetrahydrofolate | | reductase | |
| | | | | reductase 1 | | | |
| | | | | | PF00122 | E1-E2 ATPase | GO:0016020 |
| Calcium ion binding | | | | | | | |
| SOFAFFX.1248.1.S1_AT | −1.6 | Sb01g048570.1 | Os03g0128700 | similar to Calcium- | PF00036 | EF hand | GO:0005509 |
| | | | | dependent protein kinase | | | |
| | | | | isoform 11 | | | |
| | | | | | PF00036 | EF hand | GO:0005509 |
| Cytoskeleton | | | | | | | |
| SOF.4093.2.S1_AT | −1.7 | Sb01g009560.2 | Os03g0726100 | similar to Tubulin alpha- | PF00091 | Tubulin/FtsZ | N/A |
| | | | | 2/alpha-4 chain | | family, GTPase | |
| | | | | | | domain | |
| | | | | | PF03953 | Tubulin/FtsZ | GO:0043234 |
| | | | | | | family, C- | |
| | | | | | | terminal domain | |
| SOF.151.1.S1_AT | −1.7 | Sb04g037170.1 | Os02g0816500 | similar to Tubulin folding | PF02970 | Tubulin binding | GO:0005874 |
| | | | | cofactor A | | cofactor A | |
| SOF.110.1.A1_AT | −2 | Sb06g029500.1 | Os04g0629700 | similar to | PF00225 | Kinesin motor | GO:0005875 |
| | | | | OSJNBa0089N06.17 protein | | domain | |
| Regulation of nitrogen utilization | | | | | | | |
| SOF.3747.1.S1_A_AT | −2.2 | Sb03g008760.1 | Os01g0106400 | similar to Isoflavone | PF05368 | NmrA-like | GO:0006808 |
| | | | | reductase homolog IRL | | family | |
| | | | | | PF01073 | 3-beta | GO:0006694 |
| | | | | | | hydroxysteroid | |
| | | | | | | dehydrogenase/ | |
| | | | | | | isomerase family | |
| DNA binding | | | | | | | |
| SOF.4234.1.S1_A_AT | −2.4 | Sb10g002040.1 | Os06g0130900 | similar to Histone H3.3 | PF00125 | Core histone | GO:0003677 |
| | | | | | | H2A/H2B/H3/H4 | |
| SOF.5269.1.S1_AT | −1.7 | Sb02g025440.1 | Os09g0433600 | similar to Histone H4 | Pfam:N/A | Func:N/A | GO:N/A |
| Aromatic aminoacid biosynthesis | | | | | | | |
| SOF.2944.1.S1_AT | −2.8 | Sb01g033590.1 | Os07g0622200 | similar to Phospho-2- | PF01474 | Class-H DAHP | GO:0009073 |
| | | | | dehydro-3-deoxyheptonate | | synthetase | |
| | | | | aldolase 1, chloroplast | | family | |
| | | | | precursor | | | |
| Fatty acid biosynthesis | | | | | | | |
| SOF.2629.3.S1_A_AT | −3 | Sb03g012420.1 | Os01g0300200 | similar to ATP citrate lyase, | PF00549 | CoA-ligase | GO:0008152 |
| | | | | putative | | | |
| Protein ADP-ribosylation | | | | | | | |
| SOF.4942.3.S1_A_AT\|SOF.4942.2.S1_AT\|SOF.4942.1.S1_AT | −2.4\|−3.5\|−3.5 | Sb03g013840.1 | Os01g0351100 | similar to Poly [ADP-ribose] | PF00644 | Poly(ADP- | GO:0005634 |
| | | | | polymerase 2 (EC 2.4.2.30) | | ribose) | |
| | | | | (PARP-2) | | polymerase | |
| | | | | | | catalytic domain | |
| | | | | | PF02877 | Poly(ADP- | GO:0005634 |
| | | | | | | ribose) | |
| | | | | | | polymerase, | |
| | | | | | | regulatory | |
| | | | | | | domain | |
| Signal transduction | | | | | | | |
| SOF.285.1.S1_AT | −3.7 | Sb08g018765.1 | Os12g0570000 | similar to Protein spotted | PF00514 | Armadillo/beta- | N/A |
| | | | | leaf 11 | | catenin-like | |
| | | | | | | repeat | |
| Unknown function | | | | | | | |
| SOF.4866.1.S1_AT | −1.1 | Sb08g020760.1 | Os12g0604800 | similar to Tetratricopeptide | PF00515 | Tetratricopeptide | N/A |
| | | | | repeat protein, putative, | | repeat | |
| | | | | expressed | | | |
| SOF.3234.1.S1_AT | −1.1 | Sb01g011740.1 | Os03g0685500 | similar to Putative | PF06747 | CHCH domain | N/A |
| | | | | uncharacterized protein | | | |
| | | | | OSJNBb0072E24.9 | | | |
| SOF.3225.2.S1_A_AT | −1.1 | Sb02g026990.1 | Os09g0465500 | similar to Os02g0781700 | Pfam:N/A | Func:N/A | GO:N/A |
| | | | | protein | | | |
| SOFAFFX.794.1.S1_S_AT | −1.2 | Sb09g029170.1 | Os01g0652600 | similar to Putative | PF01450 | Acetohydroxy | GO:0009082 |
| | | | | uncharacterized protein | | acid | |
| | | | | | | isomeroreductase, | |
| | | | | | | catalytic | |
| | | | | | | domain | |
| SOF.849.1.A1_AT | −1.2 | Sb09g023620.1 | Os01g0818600 | similar to Unkown protein | PF00560 | Leucine Rich | GO:0005515 |
| | | | | | | Repeat | |
| SOF.5337.2.S1_AT | −1.2 | Sb01g006220.1 | Os07g0142000 | similar to Putative | PF02453 | Reticulon | GO:0005783 |
| | | | | uncharacterized protein | | | |
| SOF.4768.1.A1_AT | −1.2 | Sb01g012470.1 | Os03g0666700 | similar to Expressed protein | PF05967 | Eukaryotic | N/A |
| | | | | | | protein of | |
| | | | | | | unknown | |
| | | | | | | function | |
| | | | | | | (DUF887) | |
| SOF.2335.1.S1_AT | −1.3 | Sb03g026700.1 | Os01g0593200 | similar to Putative | PF04570 | Protein of | N/A |
| | | | | uncharacterized protein | | unknown | |
| | | | | | | function | |
| | | | | | | (DUF581) | |
| SOF.1965.1.S1_AT | −1.3 | Sb09g022110.1 | Os05g0451300 | similar to Putative | Pfam:N/A | Func:N/A | GO:N/A |
| | | | | uncharacterized protein | | | |
| SOF.3739.1.S1_S_AT | −1.4 | Sb06g026710.1 | Os04g0586200 | similar to H0307D04.13 | PF04570 | Protein of | N/A |
| | | | | protein | | unknown | |
| | | | | | | function | |
| | | | | | | (DUF581) | |
| SOF.1054.1.S1_AT | −1.4 | Sb03g042480.1 | Os01g0894700 | similar to Putative | Pfam:N/A | Func:N/A | GO:N/A |
| | | | | uncharacterized protein | | | |
| SOF.466.1.S1_AT | −1.5 | Sb07g001710.1 | Os08g0117900 | similar to Putative glycine- | Pfam:N/A | Func:N/A | GO:N/A |
| | | | | rich protein | | | |
| SOFAFFX.868.1.S1_S_AT | −1.7 | Sb02g009980.1 | Os07g0418200 | similar to Putative | Pfam:N/A | Func:N/A | GO:N/A |
| | | | | uncharacterized protein | | | |
| SOF.2471.1.S1_AT | −1.4 | Sb02g006420.1 | Os07g0211900 | similar to Putative bZIP | PF04783 | Protein of | N/A |
| | | | | protein | | unknown | |
| | | | | | | function | |
| | | | | | | (DUF630) | |
| | | | | | PF04782 | Protein of | N/A |
| | | | | | | unknown | |
| | | | | | | function | |
| | | | | | | (DUF632) | |
| SOF.2465.1.S1_AT | −1.4 | Sb02g032470.1 | Os09g0556700 | similar to Os09g0556700 | PF00856 | SET domain | GO:0005634 |
| | | | | protein | | | |
| SOF.4919.1.S1_AT | −1.5 | Sb02g022510.1 | Os09g0344800 | similar to Membrane | PF01925 | Domain of | GO:0016021 |
| | | | | protein-like | | unknown | |
| | | | | | | function DUF81 | |
| SOF.4946.2.S1_A_AT | −1.6 | Sb03g010350.1 | Os01g0265100 | similar to Putative | PF00025 | ADP- | GO:0005525 |
| | | | | uncharacterized protein | | ribosylation | |
| | | | | | | factor family | |
| | | | | | PF08477 | N/A | GO:0005622 |
| SOF.807.1.S1_AT | −1.7 | Sb02g002940.1 | Os07g0148800 | weakly similar to | PF00560 | Leucine Rich | GO:0005515 |
| | | | | Chromosome chr10 | | Repeat | |
| | | | | scaffold_43 | | | |
| SOF.4652.1.S1_AT | −1.7 | Sb03g037360.2 | Os05g0494500 | similar to | Pfam:N/A | Func:N/A | GO:N/A |
| | | | | Phosphate/phosphoenolpyruvate | | | |
| | | | | translocator protein-like | | | |
| SOF.3249.1.S1_AT | −1.8 | Sb02g043510.1 | Os03g0319300 | similar to Putative | PF03959 | Domain of | N/A |
| | | | | uncharacterized protein | | unknown | |
| | | | | | | function | |
| | | | | | | (DUF341) | |
| | | | | | PF00036 | EF hand | GO:0005509 |
| SOF.3649.1.A1_AT | −2 | Sb01g007870.1 | Os03g0751600 | similar to Expressed protein | Pfam:N/A | Func:N/A | GO:N/A |
| SOF.3476.1.S1_AT | −2.1 | Sb03g009900.1 | Os01g0257100 | similar to Putative | PF05498 | Rapid | N/A |
| | | | | uncharacterized protein | | Alkalinization | |
| | | | | | | Factor (RALF) | |
| SOF.3418.2.S1_AT\|SOF.3418.3.S1_A_AT | −2.2\|−2 | Sb01g009520.1 | Os03g0726500 | similar to Expressed protein | Pfam:N/A | Func:N/A | GO:N/A |
| SOFAFFX.1105.1.S1_AT | −2.4 | Sb06g022030.1 | Os04g0508000 | similar to | PF03005 | Arabidopsis | N/A |
| | | | | OSJNBb0002J11.24 protein | | proteins of | |
| | | | | | | unknown | |
| | | | | | | function | |
| SOF.3624.1.S1_AT | −3 | Sb03g005150.1 | Os01g0249200 | similar to Putative | PF00190 | Cupin | GO:0045735 |
| | | | | uncharacterized protein | | | |
| | | | | | PF07883 | Cupin domain | N/A |
| SOFAFFX.1040.1.S1_AT | −3.2 | Sb03g010380.1 | Os01g0265800 | similar to Putative | PF00076 | RNA | GO:0003676 |
| | | | | uncharacterized protein | | recognition | |
| | | | | | | motif. (a.k.a. | |
| | | | | | | RRM, RBD, or | |
| | | | | | | RNP domain) | |
| | | | | | PF00076 | RNA | GO:0003676 |
| | | | | | | recognition | |
| | | | | | | motif, (a.k.a. | |
| | | | | | | RRM, RED, or | |
| | | | | | | RNP domain) | |
| SOF.848.1.A1_AT | −3.5 | Sb01g016110.1 | Os03g0571900 | similar to Os03g0571900 | PF01554 | MatE | GO:0016020 |
| | | | | protein | | | |
| | | | | | PF01554 | MatE | GO:0016020 |
| SOF.5314.1.A1_AT | −3.6 | Sb04g025760.1 | Os02g0611800 | similar to Putative | PF02458 | Transferase | N/A |
| | | | | uncharacterized protein | | family | |
| SOF.2354.1.S1_A_AT | −3.9 | Sb03g025160.1 | Os01g0550300 | similar to Putative | Pfam:N/A | Func:N/A | GO:N/A |
| | | | | uncharacterized protein | | | |

The function for each gene is based on its Pfam domain and Gene Ontology (GO). The annotation of rice genes is based on RAP2 (Nucleic Acid Res. (2008) 36, D1028-1033). The expression is shown as Log2 mean ratio, with a positive or negative fold change indicating increased or decreased expression in sweet sorghum Rio with respect to grain sorghum BTx623.
\hat{ }Genes previously reported by Casu et al. (2007) are shown in red.

SUPPLEMENTAL TABLE 2
List of differentially expressed genes between grain and sweet
sorghum with no orthologous copy in a syntenic position in rice.

| Sugarcane Probe | | | | | | Gene |
|---|---|---|---|---|---|---|
| Set ID | Expression | S. bicolor ID | Sb function | Pfam | Description | ontology |
| Up-regulated | | | | | | |
| Cell wall related | | | | | | |
| Sof.383.1.S1_at\hat{ } | 1 | Sb10g006290 | Similar to | PF00107 | Zinc-binding | |
| | | | Os11g0622800 | | dehydrogenase | |
| | | | | PF08240 | Alcohol | |
| | | | | | dehydrogenase; | |
| | | | | | GroES-like | |
| | | | | | domain | |
| Chaperonin activity | | | | | | |
| Sof.1066.2.A1_x_at | 1 | Sb07g028270.1 | Similar to Heat | PF00183 | Hsp90 protein | GO:0006457 |
| | | | shock protein 82 | | | |
| | | | | PF02518 | Histidine- | GO:0005524 |
| | | | | | kinase; DNA | |
| | | | | | gyrase | |
| | | | | | B; HSP90-like | |
| | | | | | ATPase | |
| Transcription factor | | | | | | |
| Sof.4567.2.S1_a_at/ | 1.4/1.3 | Sb01g044810 | Similar to | PF00319 | SRF-type | GO:0005634 |
| Sof.4567.1.S1_at | | | putative MADS- | | transcription | |
| | | | domain | | factor (DNA- | |
| | | | transcription | | binding and | |
| | | | factor | | dimerization | |
| | | | | | domain) | |
| | | | | PF01486 | K-box region | GO:0005634 |
| Proteolysis | | | | | | |
| SofAffx.102.1.S1_at | 1 | Sb01g033620 | Similar to | PF00656 | Caspase | GO:0006508 |
| | | | Os03g0388900 | | domain | |
| Nucleic acid binding | | | | | | |
| Sof.3151.2.S1_a_at | 1 | Sb04g025670 | Similar to | PF00076 | RNA | GO:0003676 |
| | | | putative | | recognition | |
| | | | uncharacterized | | motif. (a.k.a. | |
| | | | protein | | RPM, RBD or | |
| | | | | | RNP domain) | |
| Unknown function | | | | | | |
| Sof.405.2.S1_a_at | 5.7 | Sb09g013990 | Similar to | | Similar to | |
| | | | putative | | Saposin type B | |
| | | | uncharacterized | | protein | |
| | | | protein | | | |
| Sof.4787.1.A1_at | 1 | Sb01g026550.1 | Similar to | PF00561 | Alpha/beta | |
| | | | Os10g0135600 | | hydrolase fold | |
| SofAffx.403.1.S1_at | 1.3 | Sb10g002980 | Similar to | | Unknown | |
| | | | putative | | | |
| | | | uncharacterized | | | |
| | | | protein | | | |
| Sof.22.1.S1_at | 3.2 | Sb01g041540 | Similar to Purple | | Unknown | |
| | | | acid phosphatase | | | |
| | | | 1, putative, | | | |
| | | | expressed | | | |
| Sof.4906.1.S1_at | 1 | Sb01g023540 | Similar to | | Unknown | |
| | | | expressed protein | | | |
| Down-regulated | | | | | | |
| Cell wall related | | | | | | |
| Sof.1987.1.S1_at | −1.5 | Sb04g011550 | Putative | PF01073 | 3-beta | G0:0006694 |
| | | | cinnamyl alcohol | | hydroxysteroid | |
| | | | dehydrogenase | | dehydrogenase/ | |
| | | | | | isomerase | |
| | | | | | family | |
| | | | | PF01370 | NAD | GO:0044237 |
| | | | | | dependent | |
| | | | | | epimerase/ | |
| | | | | | dehydratase | |
| | | | | | family | |
| | | | | PF07993 | Male sterility | |
| | | | | | protein | |
| Sof.1519.2.S1_at | −1.4 | Sb02g006330 | Putative | PF03345 | Dolichyl- | GO:0005789 |
| | | | Dolichyl- | | diphospho- | |
| | | | diphospho- | | oligosaccharide- | |
| | | | oligosaccharide- | | protein | |
| | | | protein | | glycosyltransferase | |
| | | | | | 48 kD | |
| | | | | | subunit | |
| Sof.3569.2.S1_at | −1.1 | Sb06g015880 | Xyloglucan | PF00722 | Glycosyl | GO:0005975 |
| | | | endo- | | hydrolases | |
| | | | transglycosylase/ | | family 16 | |
| | | | hydrolase | | | |
| | | | precursor | | | |
| | | | | PF06955 | Xyloglucan | GO:0048046 |
| | | | | | endo- | |
| | | | | | transglycosylase | |
| | | | | | (XET) C- | |
| | | | | | terminus | |
| Sof.4258.2.S1_a_at | −1.5 | Sb05g027350 | Putative | | Unknown | |
| | | | Xylanase | | | |
| | | | inhibitor | | | |
| Sof.4229.2.S1_a_at\hat{ } | −1.1 | Sb02g029640 | Similar to | PF00232 | Glycosyl | GO:0005975 |
| | | | Glycoside | | hydrolase | |
| | | | hydrolase family | | family 1 | |
| | | | 1 protein | | | |
| Sof.478.2.S1_at | −1.5 | Sb02g004660 | Similar to | PF00704 | Glycosyl | GO:0005975 |
| | | | Putative | | hydrolases | |
| | | | Xylanase | | family 18 | |
| | | | inhibitor protein | | | |
| | | | precursor | | | |
| Sof.3100.1.S1_at | −2 | Sb04g026520 | Similar to | PF00221 | Phenylalanine | GO:0009058 |
| | | | Phenylalanine | | and histidine | |
| | | | and histidine | | ammonia-lyase | |
| | | | ammonia-lyase | | | |
| Sof.3641.1.A1_at | −1.5 | Sb02g037840 | Similar to plasma | PF00141 | Peroxidase | GO:0006979 |
| | | | membrane bound | | | |
| | | | peroxidase | | | |
| Acyl CoA binding | | | | | | |
| SofAffx.816.1.S1_at | −2.6 | Sb07g004260 | Similar to Acyl | PF00887 | Acyl CoA | GO:0000062 |
| | | | CoA binding | | binding protein | |
| | | | protein | | | |
| Cystein protease | | | | | | |
| inhibitor activity | | | | | | |
| SofAffx.772.1.S1_s_at | −3.2 | Sb03g037370 | Similar to | PF00031 | Cystatin | GO:0004869 |
| | | | Cystatin | | domain | |
| Tanslation/Ribosome | | | | | | |
| Sof.3035.1.S1_at | −1.3 | Sb08g015010 | Similar to | PF01092 | Ribosomal | GO:0005840 |
| | | | Ribosomal | | protein S6e | |
| | | | protein S6 RPS6- | | | |
| | | | 1 | | | |
| Electron transport | | | | | | |
| Sof.5340.1.S1_at | −1.7 | Sb01g047640 | Similar to | PF00067 | Cytochrome | GO:0006118 |
| | | | Cytochrome | | P450 | |
| | | | P450 family | | | |
| | | | protein, | | | |
| | | | expressed | | | |
| Proteolysis | | | | | | |
| Sof.15.2.S1_a_at | −1.4 | Sb08g020950 | Weakly similar | PF00450 | Serine | GO:0006508 |
| | | | to serine | | carboxypeptidase | |
| | | | carboxypeptidase | | | |
| Unknown function | | | | | | |
| SofAffx.778.1.S1_s_at/ | −1.2 | Sb09g006610 | Putative | PF00069 | Protein kinase | GO:0006468 |
| Sof.258.1.S1_at | | | uncharacterized | | domain | |
| | | | protein | | | |
| | | | | PF07714 | Protein | GO:0006468 |
| | | | | | tyrosine kinase | |
| Sof.3156.2.S1_a_at | −1.5 | Sb09g0200860 | Unknown protein | PF03083 | MtN3/saliva | |
| | | | | | family | |
| Sof.3284.1.S1_at | −2.7 | Sb10g000510 | Putative | PF00234 | Protease | |
| | | | uncharacterized | | inhibitor/seed | |
| | | | protein | | storage/LTP | |
| | | | | | family | |
| Sof.4668.1.S1_at | −1.6 | Sb07g006900 | Similar to | | Unknown | |
| | | | putative | | | |
| | | | uncharacterized | | | |
| | | | protein | | | |
| Sof.498.1.A1_at | −2.9 | Sb02g003020 | Similar to | PF07967 | C3HC zinc | GO:0005634 |
| | | | express protein | | finger-like | |

The function for each gene is based on its Pfam domain and Gene Ontology (GO). The expression is shown as Log2 mean ratio, with a positive or negative fold change indicating increased or decreased expression in sweet sorghum Rio with respect to grain sorghum BTx623. Genes previously reported by Casu et al. (2007) are shown in red.

Supplemental TABLE 3
Primer sequences used in qRT-PCR reactions

| S. bicolor gene ID/Probe Set ID | Forward | Reverse |
|---|---|---|
| Sb09g013990.1/Sof.405.2.S1_a_at | 5′TGCTGGATCACAAATCCTCA3′ | 5′ATAGCGCCTGGACTCCTTTT3′ |
| Sb09g028490.1/Sof.3590.1.S1_at | 5′CAGTTCAGCGAGTTCAAGCA3′ | 5′TCACGCAGTAGAGCACCATC3′ |
| Sb03g040060.1/Sof.90.1.S1_at | 5′GCCAAGGAGATATGGGACAT3′ | 5′AGCACCGTGGGTCATTATTC3′ |
| Sb09g005280.1/Sof.5033.1.S1_at | 5′TTGTCTGGTCCATCCTCCTC3′ | 5′TTTCCCATCTAGCCTCCTCA3′ |
| Sb07g001320.1/Sof.3644.2.S1_a_at | 5′CCTGAAGCAAAACAACGTCA3′ | 5′GGGTTCCGGTAGAACATGAA3′ |
| Sb04g005210.1/Sof.4734.1.S1_at | 5′ACCGAAGGCTCTGAAGTCAC3′ | 5′GGGGATGGATTCAGTGAAGA3′ |
| Sb01g033060.1/Sof.4165.1.S1_s_at | 5′CTTTTCCCTGGGTTTCCTTC3′ | 5′TCCCTCTCAACCGACTCAAC3′ |
| Sb01g002050.1/Sof.1587.3.A1_a_at | 5′TGACTGCAATATTGGGCAAA3′ | 5′AACTTTCTGTTCGGCTCACC3′ |
| Sb03g003190.1/Sof.4315.1.S1_at | 5′GCCATGGGTGCTTACCATAG3′ | 5′CCAAGCCTCGTTTTGGTTAT3′ |
| Sb03g039520.1/Sof.1021.1.A1_at | 5′CGATCTTCCCAAATGCTGAT3′ | 5′GTCCAGGTCAGCTAGGAACG3′ |
| Sb07g021680.1/Sof.3629.1.S1_at | 5′GCGTGAGCTAGAGGGAGATG3′ | 5′CAGCCAGCGAACAAACACTA3′ |
| Sb03g033250.1/Sof.1594.1.S1_at | 5′TGCATGTACAGCCCCATTTA3′ | 5′GCAGAACAGGACGTGAAACA3′ |
| Sb03g041450.1/Sof.4934.1.S1_at | 5′AGGCCTGTCTGAACACCAAT3′ | 5′CATGGGCACAGTTGTAGTGG3′ |
| Sb01g018400.1/Sof.3244.1.S1_a_at | 5′CACTCATCATTCTCGGCTCA3′ | 5′CACACTATGGACTCCGCTCA3′ |

Primers were designed based on the sequence from sorghum genes with homology to sugarcane Probe set IDs. Forward primer sequences are SEQ ID NOs: 1, 3, 5, 7, 9, 11, 13, 15, 17, 19, 21, 23, 25, and 27, respectively, from top to bottom and reverse primer sequences are SEQ ID NOs: 2, 4, 6, 8, 10, 12, 14, 16, 18, 20, 22, 24, 26, and 28, respectively, from top to bottom.

### EXAMPLE 2

### Comparison of Flowering Time to Brix Degree

Sweet sorghum and sugarcane are closely related grass species that accumulate sugars in their stems. These sugars can be fermented to ethanol. Sugar accumulation in both species is maximized at the time of flowering. Sorghum is considered as a short day plant, which means that it flowers earlier under short days (defined as 10 hours of light and 14 hours of dark), than under long days (defined as 16 hours of light and 8 hours of dark). With the introduction of sweet sorghum as a biofuel crop, the development of cultivars fully adapted to different geographic regions varying in day length and climate is needed.

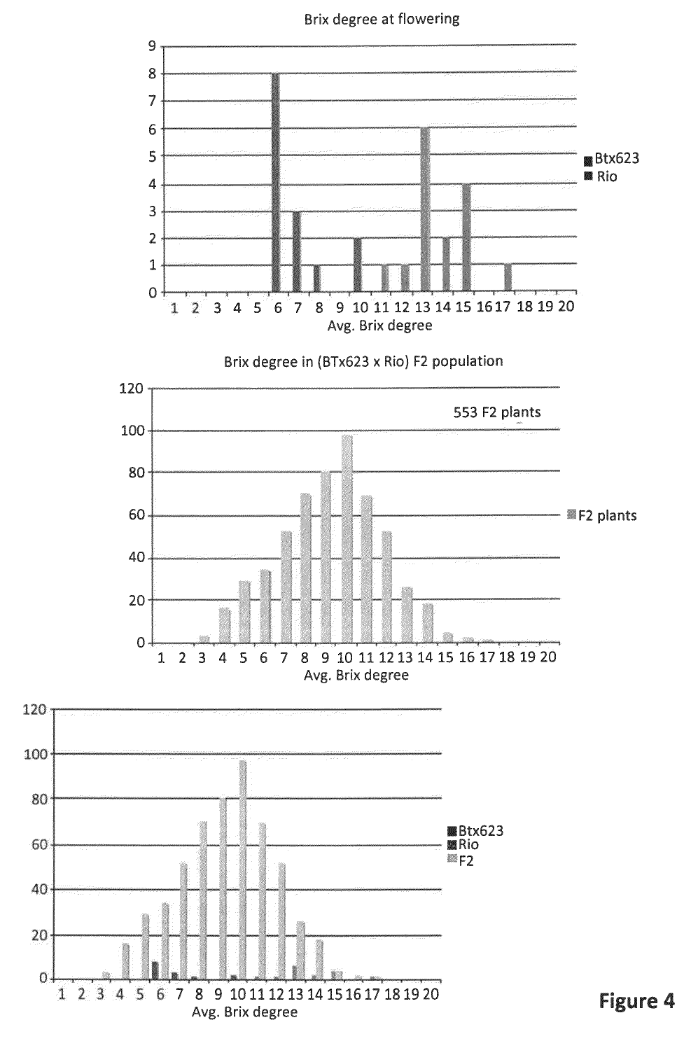

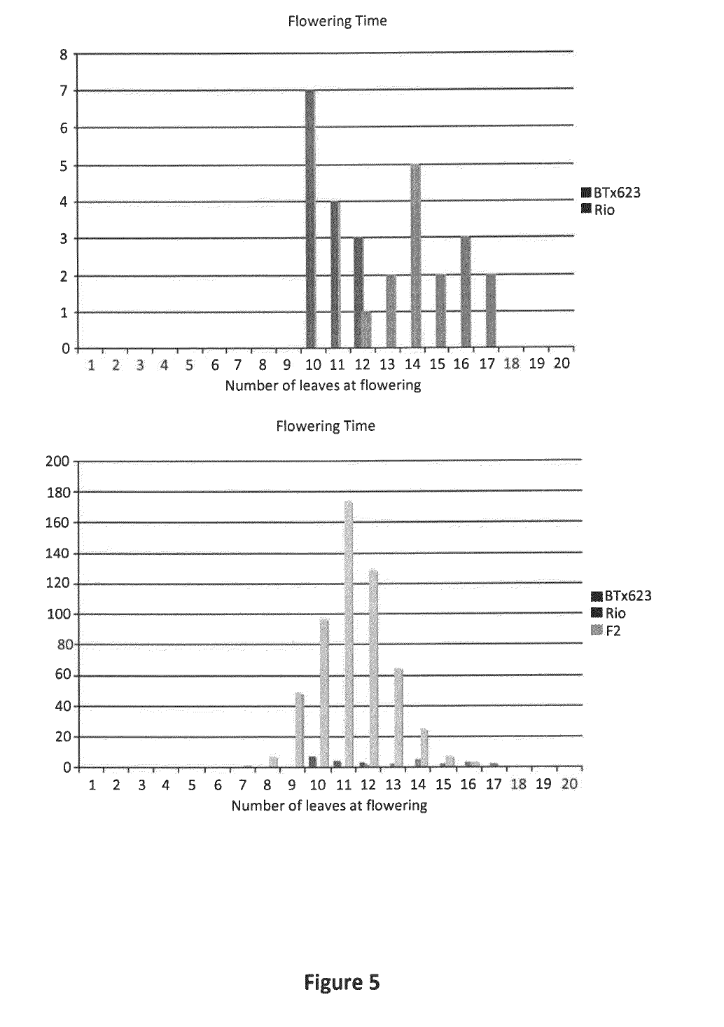

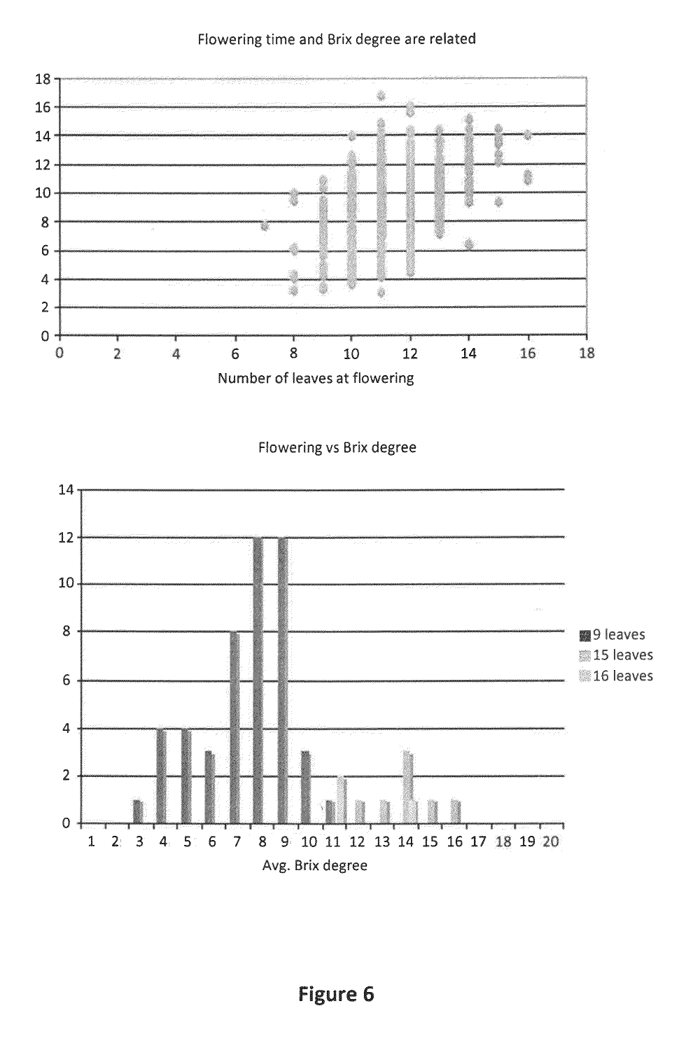

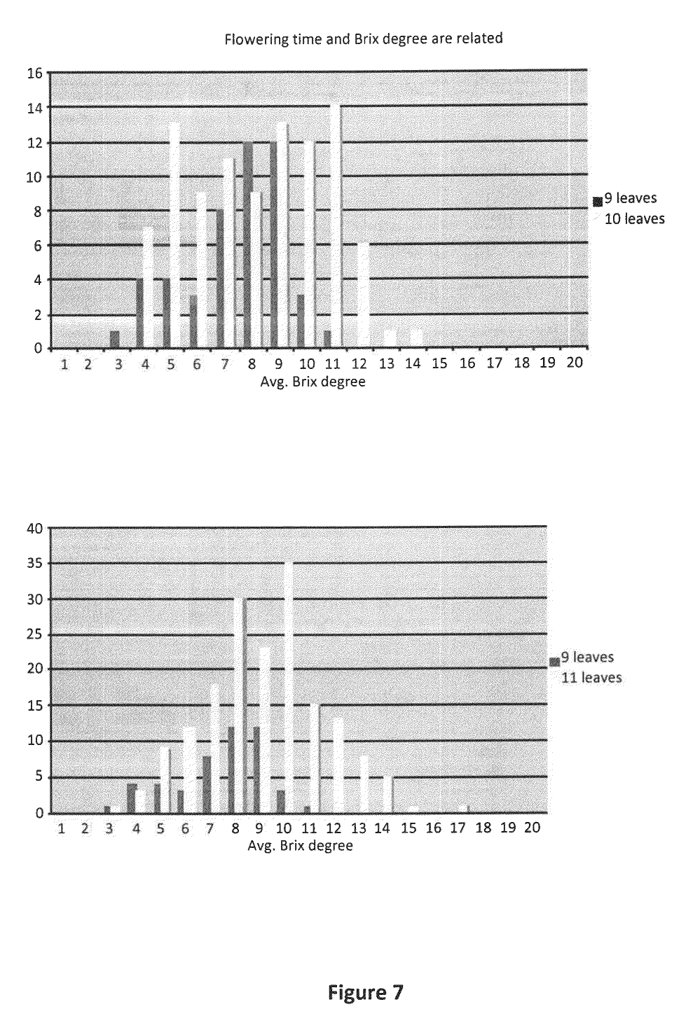

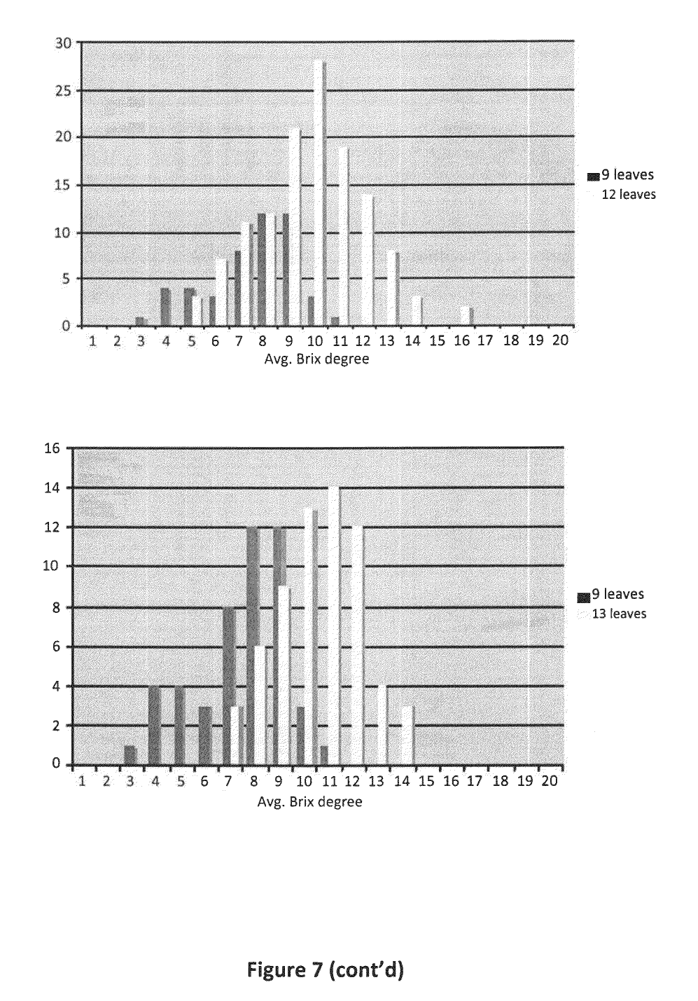

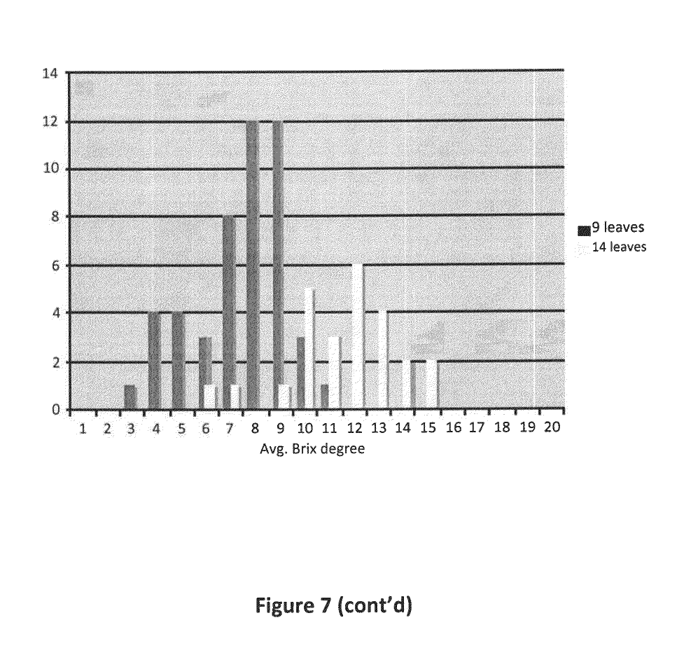

Our preliminary data suggests a link between flowering time and sugar accumulation in sorghum. When sugar accumulation is measured in F2 plants derived from the cross of grain sorghum (low sugar and early flowering) with sweet sorghum (high sugar and late flowering), the stems of late flowering F2 plants displayed higher sugar accumulation than the stems of early flowering F2 plants. The results of this study are set out in FIGS. 4 to 7. For this reason, it is important to investigate the co-segregation of flowering time genes and sugar content in an F2 mapping population.

This is consistent with a recent report by Seth C. Murray, Arun Sharma, William L. Rooney, Patricia E. Klein, John E. Mullet, Sharon E. Mitchell, and Stephen Kresovich. Genetic Improvement of Sorghum as a Biofuel Feedstock: I. QTL for Stem Sugar and Grain Nonstructural Carbohydrates Crop Science. 2008 48: 2165-2179, (“Murray et al. 2008a”), where they also described that a specific genomic region on chromosome 6 (known as Quantitative trait locus or QTLs) influence both flowering time and the amount of sugars in stem juice. In the report from Murray et al. 2008a, the authors used a Recombinant Inbred Line (RIL) derived from the cross of Btx623 and Rio; the same parental lines we used in our study. Although they described a relationship between flowering time and sugar content in sorghum they do not state the potential importance of modifying flowering time to adapt sorghum to specific geographic areas in order to improve the sugar content yield for biofuel production as we do. Furthermore, the F2 mapping population that we have created will allow us to identify genes involved in flowering that may have an impact (direct or indirectly) in sugar content and thus can be used for biofuel applications.

We have found that the expression level of two micro-RNA genes termed microRNAs 172a and c (miR172a and miR172c) co-segregate with sugar content in F2 plants. In other words, we found that the relative expression level of miR172a and miR172c in Btx623 is twice as high as in Rio. When the expression of these two microRNA genes was analyzed in F2 plants displaying low Brix and early flowering (resembling the Btx623 parent phenotype) and in F2 plants with high Brix and late flowering (resembling the Rio parent) we found that miR172a and miR172c expression level is also twice as high in the low Brix and early flowering F2s as compared to high Brix and late flowering F2 plants. This means that the expression level difference in miR172a and miR172c between BTx623 and Rio is inherited in the F2 generation.

This finding means that miR172a and miR172c (and the target genes they regulate), could be used to manipulate the flowering time, sugar content and biomass of sorghum to produce plants fully adapted to different geographic in where biofuel production may be required. A statistical summary for miRNA is set forth below.

| | | Number of | relative num- | Total number of se- |
|---|---|---|---|---|
| miRNA | Library | sequences | ber [%] | quences in library |
| sbi-MIR172a | Btx | 37,769 | 2.643% | 1,429,021 |
| sbi-MIR172a | Rio | 28,459 | 1.229% | 2,315,148 |
| sbi-MIR172a | Low Brix | 124,587 | 1.562% | 7,975,867 |
| sbi-MIR172a | High Brix | 75,185 | 0.741% | 10,139,788 |
| sbi-MIR172c | Btx | 37,173 | 2.601% | 1,429,021 |
| sbi-MIR172c | Rio | 28,113 | 1.214% | 2,315,148 |
| sbi-MIR172c | Low Brix | 12,0975 | 1.517% | 7,975,867 |
| sbi-MIR172c | High Brix | 72,973 | 0.720% | 10,139,788 |

### EXAMPLE 3

### Molecular Markers for Sweet Sorghum Based on Microarray Expression Data, SFP Discovery in Sorghum

In Example 3, using an Affymetrix sugarcane genechip we previously identified 154 genes differentially expressed between grain and sweet sorghum set forth above in Example 1. Although many of these genes have functions related to sugar and cell wall metabolism, dissection of the trait requires genetic analysis. Therefore, it would be advantageous to use microarray data for generation of genetic markers, shown in other species as single feature polymorphisms (SFPs). As a test case, we used the GeSNP software to screen for SFPs between grain and sweet sorghum. Based on this screen, out of 58 candidate genes 30 had SNPs, from which 19 had validated SFPs. The degree of nucleotide polymorphism found between grain and sweet sorghum was in the order of one SNP per 248 base pairs, with chromosome 8 being highly polymorphic. Indeed, molecular markers could be developed for a third of the candidate genes, giving us a high rate of return by this method.

Introduction

The development of molecular markers is essential for marker-assisted selection in plant breeding as well as to understand crop domestication and plant evolution (Varshney et al. 2005). Single nucleotide polymorphisms (SNPs) have become the marker of choice because of their abundance and uniform distribution throughout the genome (Gupta et al. 2008; Varshney et al. 2005; Zhu and Salmeron 2007). Around 90% of the genetic variation in any organism is attributed to SNPs (Varshney et al. 2005; Zhu and Salmeron 2007). They are discovered from genomic or EST sequences available in databases or through sequencing of candidate genes, PCR products or even whole genomes (Varshney et al. 2005; Zhu and Salmeron 2007).

Recent studies have described the use of transcript abundance data from RNA hybridizations to Affymetrix microarrays to discover genetic polymorphisms that can be utilized as markers for genotyping in mapping populations (Borevitz and Chory 2004; Gupta et al. 2008; Hazen and Kay 2003; Shiu and Borevitz 2008; Zhu and Salmeron 2007). In an Affymetrix chip, each gene is represented by 11 different 25-bp oligonucleotides that cover features of the transcribed region of that gene. Each of these features is described as a perfect match (PM) and mismatch (MM) oligonucleotide. The PM exactly matches the sequence of a standard genotype whereas the MM differs from the PM by a single base substitution at the central, 13th position (Borevitz and Chory 2004; Hazen and Kay 2003; Zhu and Salmeron 2007).

A new aspect of this approach is to discover sequence polymorphisms in cultivars or variants of species, where one of them has been sequenced, but where no sequence information is yet available form the other ones. Here, the hybridization data from microarrays not only measure differential gene expression, but also can yield information on sequence variation between two inbred lines. If two genotypes differ only in the amount of mRNA in a particular tissue, this should result in a relatively constant difference in hybridization throughout the eleven features. On the other hand, if the two genotypes contain a genetic polymorphism within a gene that coincides with one of the particular features, this will produce differential hybridization for that single feature. Such differences have been described as single-feature polymorphisms (SFPs) (Borevitz and Chory 2004; Borevitz et al. 2003; Hazen and Kay 2003; Zhu and Salmeron 2007). Thus, expression microarrays hybridized with RNA are able to provide us not only with phenotypic (variation in gene expression) but also with genotypic (marker) data (Zhu and Salmeron 2007). If two genotypes differ in the expression level of a particular gene, we can consider it as an expression level polymorphism or (ELP). Both, ELPs and SFPs are dominant markers and can be mapped as alleles in segregating populations (genetical genomics) and ELPs can be considered as traits to determine expression QTLs or e-QTLs (Coram et al. 2008; Jansen and Nap 2001).

In Arabidopsis, SFPs have been used for several purposes such as mapping clock mutations through bulked segregant analysis (Hazen et al. 2005), the identification of genes for flowering QTLs (Werner et al. 2005), high-density haplotyping of recombinant inbred lines (RILs) (West et al. 2006) and natural variation in genome-wide DNA polymorphism (Borevitz et al. 2007). In plant species of agronomic importance, SFPs have been utilized to identify genome-wide molecular markers in barley and rice (Kumar et al. 2007; Potokina et al. 2008; Rostoks et al. 2005) as well as markers linked to Yr5 stripe rust resistance in wheat (Coram et al. 2008). However, an impediment to SFP discovery in crop plants based on DNA hybridization to Affymetrix expression arrays could be the size of gene families (Borevitz et al. 2003; Varshney et al. 2005; Zhu and Salmeron 2007). Because the coding regions of many gene clusters that arose by tandem gene amplification are quite conserved hybridization-based approaches would not be sufficient to distinguish between allelic and paralogous copies (Xu and Messing 2008). Therefore, one would have to limit this analysis to low-copy genes. On the other hand, this approach does not aim at identifying candidate genes directly, but rather linked genetic markers.

An area where gene discovery has become of general interest is the utilization of biomass for the production of alternative fuels. Because desirable traits for biofuel crops are very complex and involve many genes from different pathways, it becomes necessary to take genetic approaches to identify key genes so that molecular breeding can be employed to make performance improvements. The most successful biofuel crop today is sugarcane. However, it cannot be grown in moderate climate. Maize, which is a major biofuel crop in the US, has a much lower yield of bioethanol per acreage than sugarcane, requires high input costs, and is a major food and feed source. A crop that bridges between the two is the close relative, sorghum. Sorghum tolerates harsher environmental conditions than sugarcane and maize, has a higher disease resistance than maize, and has a high stem-sugar variant, sweet sorghum, which has potential yields of bioethanol like sugarcane. Moreover, sweet sorghum can be crossed with grain sorghum so that genetic analysis could uncover key regulatory factors that would increase sugar and decrease lignocellulose in the biomass. Therefore, sorghum could be used to identify both SFPs and ELPs linked to high sugar content.

We have recently reported the hybridization of RNAs derived from the stems of grain and sweet sorghum onto the sugarcane Affymetrix genechip (Calviño et al. 2008). A previous study demonstrated that cross-species hybridization did not affect the reproducibility of the microarray experiment (Cáceres et al. 2003). Moreover, an Affymetrix soybean genome array has been used to identify SFPs in the closely related species cowpea (Das et al. 2008).

Here, we have asked the question whether we could use the sugarcane chip analysis to extend the cross-species concept in SFP discovery in the grasses. We report the identification of SFPs in 58 sorghum genes by using the recently developed software GeSNP (Greenhall et al. 2007). These genes were described in our previous study to be differentially expressed between grain and sweet sorghum (Calviño et al. 2008). The utility of GeSNP has been successfully tested for SFP discovery in mice, humans and chimpanzees (Greenhall et al. 2007) but there is no report on plants yet. In order to experimentally validate the SFPs identified in sorghum, we sequenced fragments from 58 genes and found SNPs in 30 of them, out of which 19 genes had a validated SFP. Furthermore, we develop molecular markers based on the SNPs found. The high experimental validation rate of SNPs of 50% of the candidate genes shows the potential of this method for the development of molecular markers and in principal the applicability to any trait of interest.

Results

SFP Discovery and Validation from Differentially Expressed Genes in Sorghum

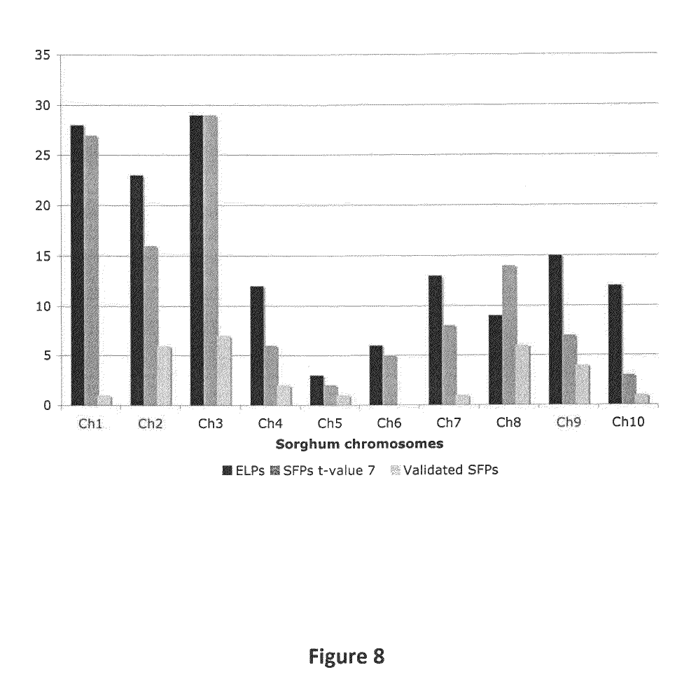

Previously, we reported the use of an Affymetrix genechip from sugarcane to identify differentially expressed genes in the stem of grain and sweet sorghum (Calviño et al. 2008). Such a cross species hybridization (CSH) approach allowed us to identify 154 genes harboring expression level polymorphisms (ELPs) between grain and sweet sorghum. In order to discover single feature polymorphisms (SFPs) within these genes as well, we uploaded the sugarcane Affymetrix CEL files previously obtained into the GeSNP software. Indeed we found that from 154 genes, 57 harbored a SFP with a t-value≧7 (FIG. 8 and Table 4). Based on existing data (Greenhall et al. 2007) we adopted a t-value of seven or higher as a threshold. Chromosomes 1, 2, and 3 had the highest number of genes displaying both ELPs and SFPs, whereas chromosomes 5 and 6 had the lowest number of ELPs and SFPs, respectively (FIG. 8).

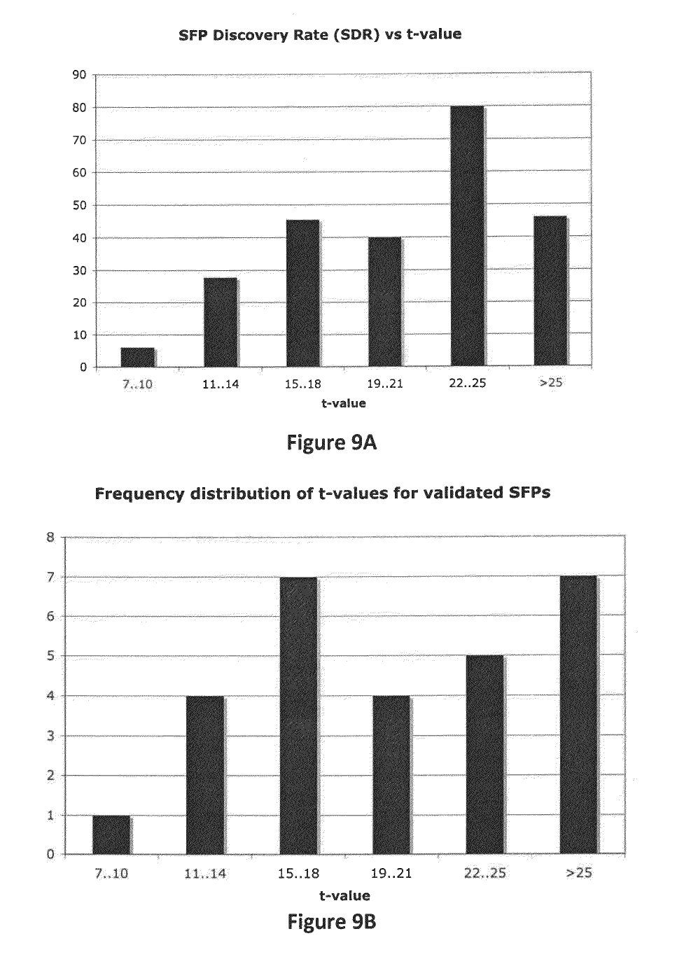

In order to validate the SFPs discovered and calculate the SFP discovery rate (SDR) of the GeSNP software, we cloned and sequenced the 57 genes harboring both ELPs and SFPs in addition to one gene harboring only SFPs (see below) from sweet sorghum Rio, and aligned the sequences against the BTx623 reference genome. The software predicted a total of 125 SFPs (on average ˜2 per gene) and we could experimentally validate 32 of them (Table 4). We calculated the SDR as 25.6% (SDR=[Validated SFPs/Total SFPs]×100). As expected, the SDR was dependent on the t-value, with the lowest SDR (less than 10%) at t-values between 7 and 10, and the highest SDR (80%) with t-values from 22 to 25 respectively (FIG. 9A).

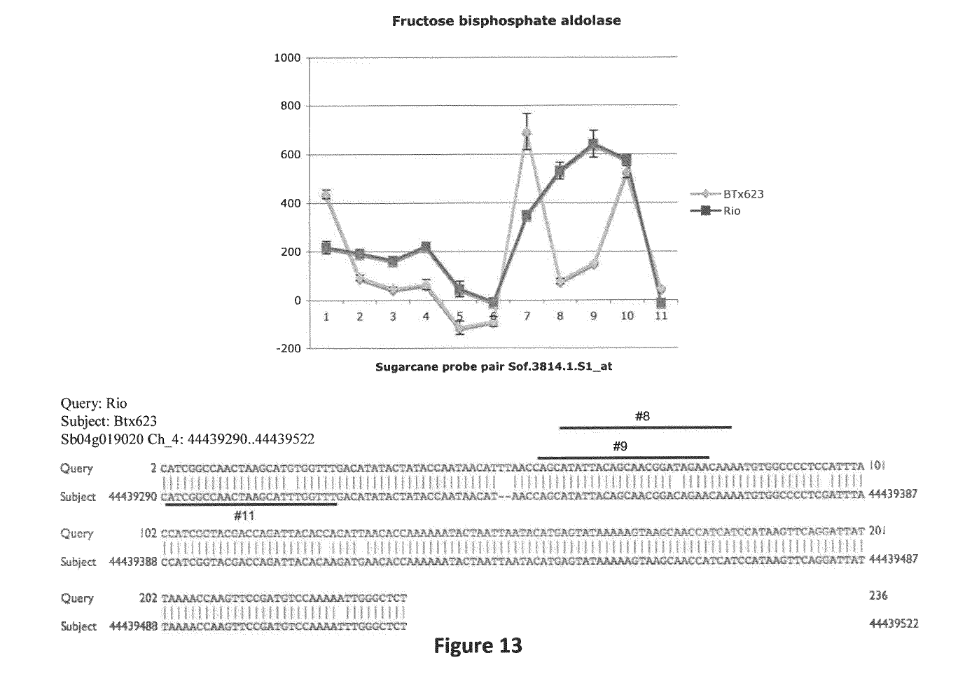

Besides SFPs identified in genes that are differentially expressed, the GeSNP software also detected SFPs in genes that did not show differential expression under our experimental conditions (data not shown). Considering the high success rate of SNPs discovered in genes having both, SFPs and ELPs, we extended our screen to genes that have predicted SFPs with t-values of 22 to 25 but no ELP. This analysis allowed us to identify 37 sugarcane probe pairs that matched the sorghum genome sequence and have a high probability of representing SNPs in genes that have no ELPs between BTx623 and Rio but were expressed in the stem (see Table 5). For example, one of the sugarcane probe pairs (Sof.3814.1.S1_at) matched a sorghum gene coding for fructose bisphospate aldolase. Since the protein product of this gene has a role in the sucrose and starch metabolic pathway (our trait of interest), we cloned and sequenced the fragment containing the SFPs. As it is shown in FIG. 13, we found 6 SNPs, two of which were recognized by three sugarcane probe pairs. This result indicates that our approach is able to efficiently detect SNPs. From the 58 genes that were sequenced, 19 genes (33%) had a validated SFP and 11 genes (19%) harbored SNPs outside the probe pairs, at different location than the one predicted by GeSNP. Therefore, the total SNP detection rate was 52%. A list of genes with validated SFPs as well as the nature of the nucleotide change/s is provided in Table 6.

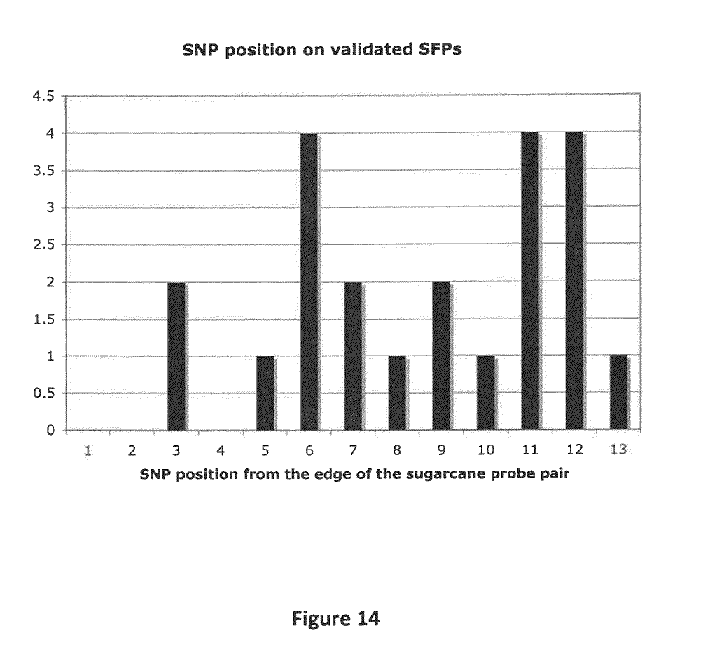

Most of the validated SFPs had probe pairs with t-values from 15 to 18 and greater than 25 (FIG. 9B). Since the SFP validation depends on the SNP position along the probe pair (Rostoks et al. 2005), we analyzed the SNP position from the edge of the sugarcane probe pair for those genes with validated SFPs (FIG. 14). We found that from a total of 22 probe pairs (probes that recognized the same SNP were not counted), 19 of them recognized a SNP between the 6^{th} and the 13^{th} position.

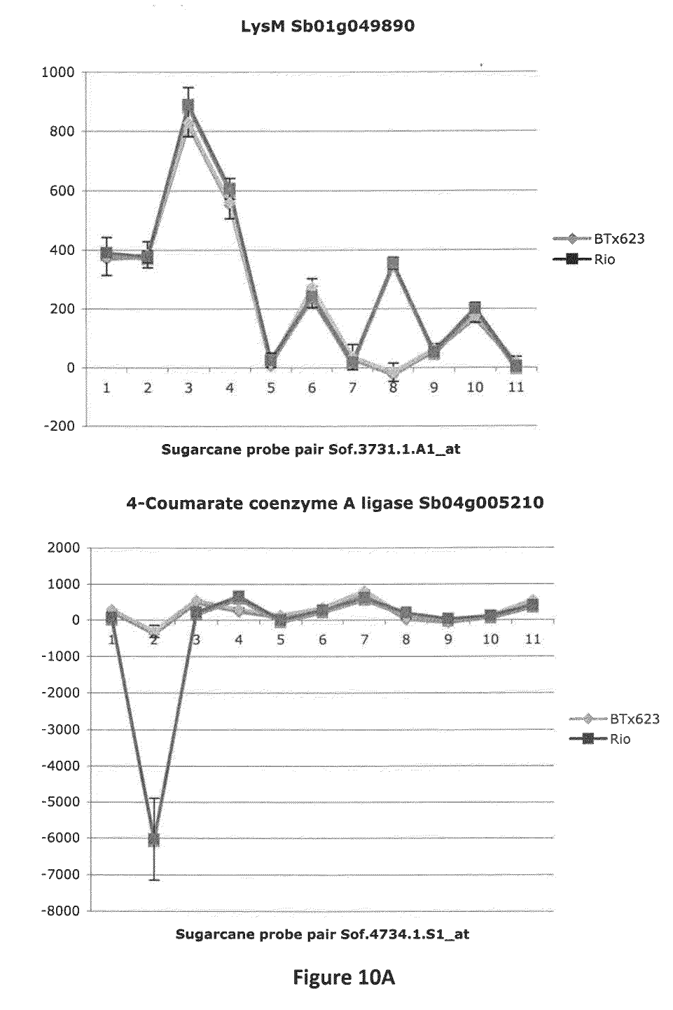

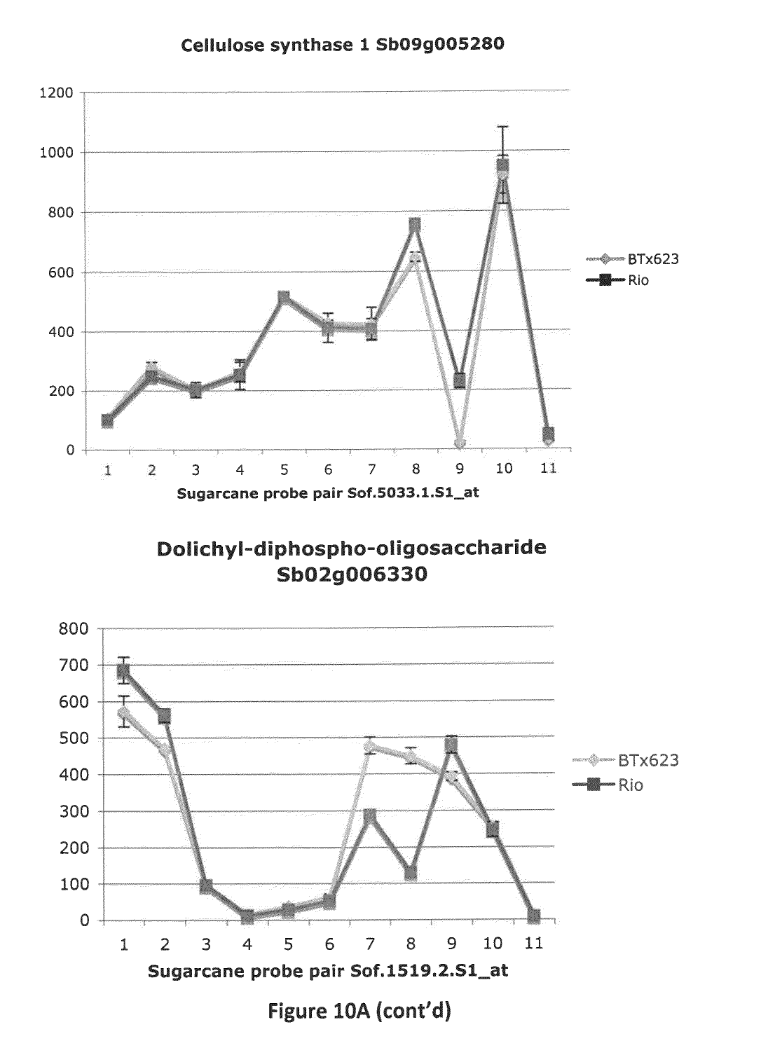

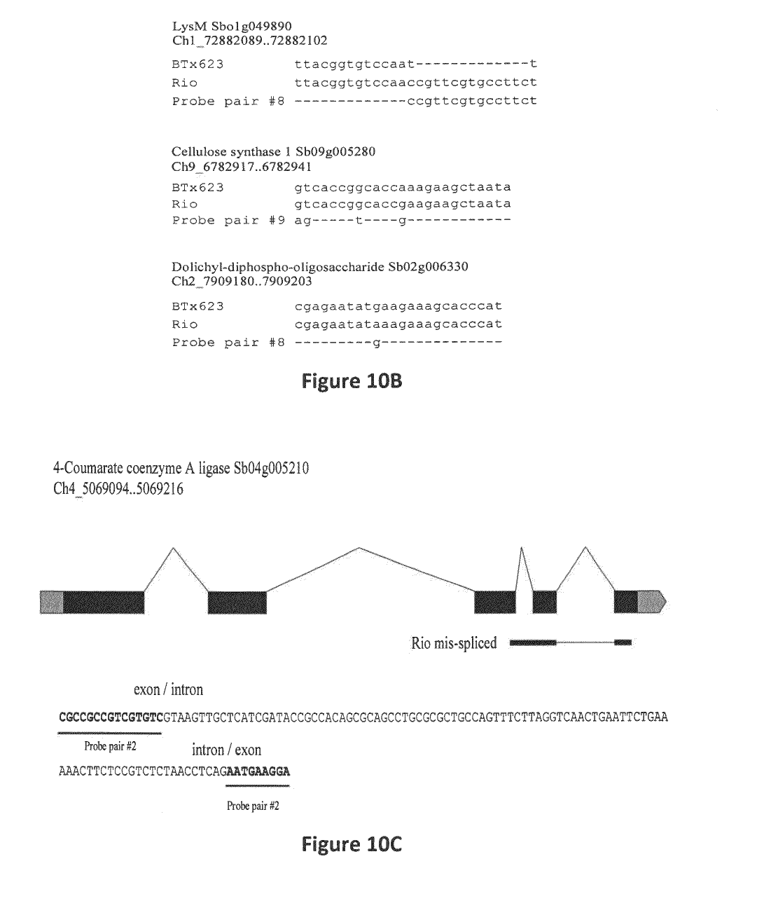

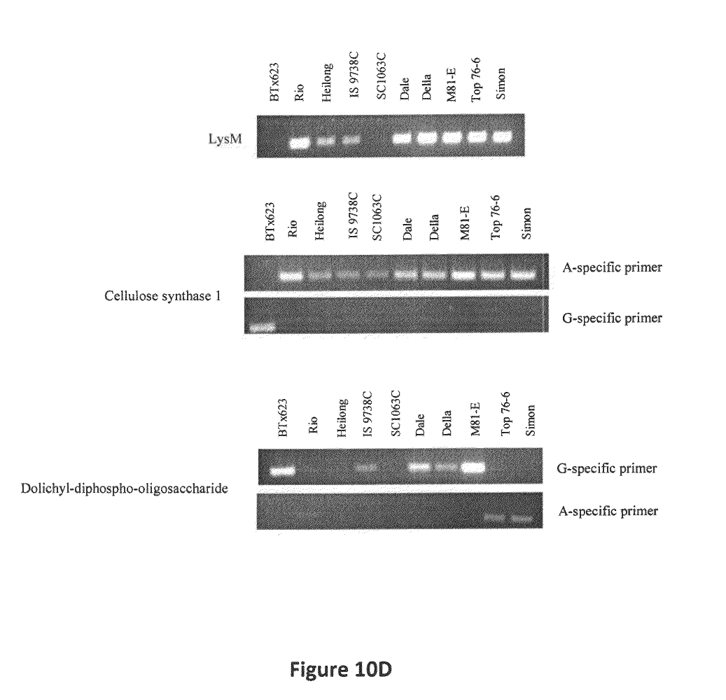

With regard to genes involved in our traits of interest, that is sugar accumulation and cell wall metabolism, we validated SFPs for 5 of them (FIG. 10 and FIG. 13). The SFPs in the cellulose synthase 1 and dolichyl-diphospho-oligosaccharide genes was based on a SNP, whereas the SFP in the LysM gene was due to a 13 bp indel (FIGS. 10A and 10B). This indel allowed us to develop an allele specific PCR marker (FIG. 10D). In the case of the 4-coumarate coenzyme A ligase gene, the SFP was based on a mis-spliced intron in Rio (FIG. 10C).

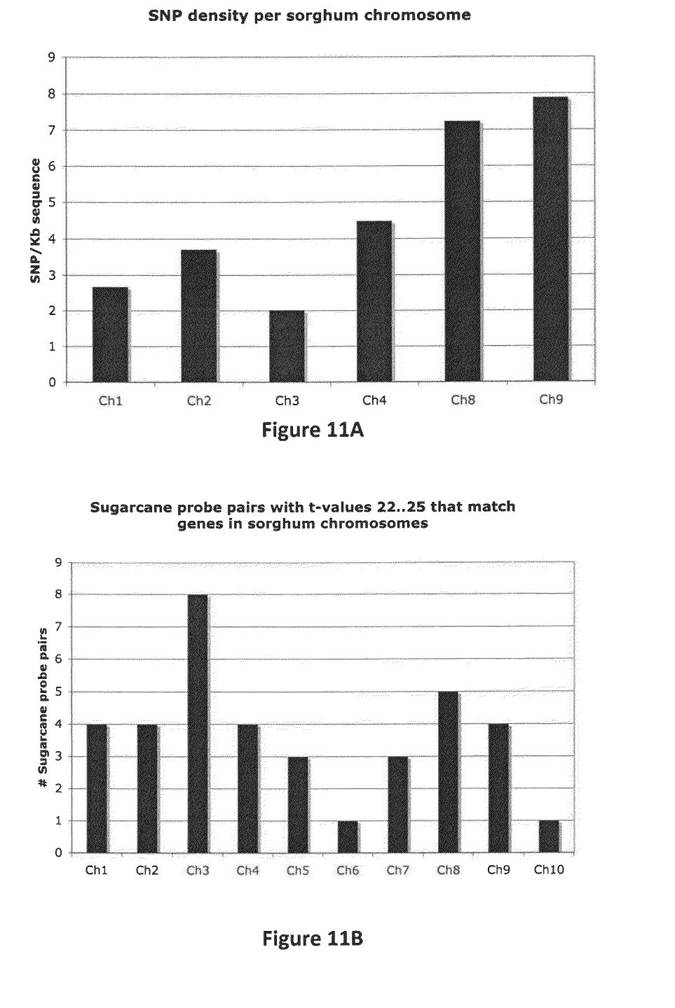

To calculate the number of SNPs per total sequence length, we determined the genome size of the Rio line by flow cytometry. The Rio line appeared to have the same genome size than the sequenced BTx623 (data not shown). Based on 87 SNPs in 21,612 bp of sequence from both parental lines, we concluded that there is an average of one SNP every 248 base pairs of sequence between BTx623 and Rio. Taking in consideration that the genome size is in the order of 730 Mbp (Paterson et al. 2009), we suggest that 2,938,800 SNPs could exist between grain sorghum BTx623 and sweet sorghum Rio and that at least 0.4% of the genome could be polymorphic between the two lines. We also looked at the SNP density per sorghum chromosome in order to see if there is any difference among them. Surprisingly, we found that the level of polymorphism is higher for chromosomes 8 and 9 and lower for chromosome 3 compared to the average SNP density per Kb of sequence (4 SNPs/Kbp) (FIG. 11A). However, if we consider the frequency of probe pairs with t-values between 22 and 25 for each sorghum chromosome as it is shown in FIG. 11B, chromosome 3 had the highest number of probes. On the other hand, chromosome 8 had the second highest number of probes with t-values between 22 and 25 together with a high SNP density (FIGS. 11A and 11B). This might suggest an unusual level of polymorphism for this chromosome between BTx623 and Rio. However, we have not sufficient data (genes sequenced) to test whether the SNP density differences among the chromosomes are statistically significant.

Sorghum genes harboring validated SFPs allowed us to investigate if such nucleotide substitutions were conserved or not within grain sorghum BTx623, sweet sorghum Rio, and sugarcane. Indeed, we found that from 22 SNPs discovered through 28 validated SFPs (one sugarcane probe pair can recognize more than one SNP), 15 of them were conserved between BTx623 and sugarcane whereas only 7 SNPs were conserved between Rio and sugarcane (Table 6).

Development of Molecular Markers Based on Validated SFPs

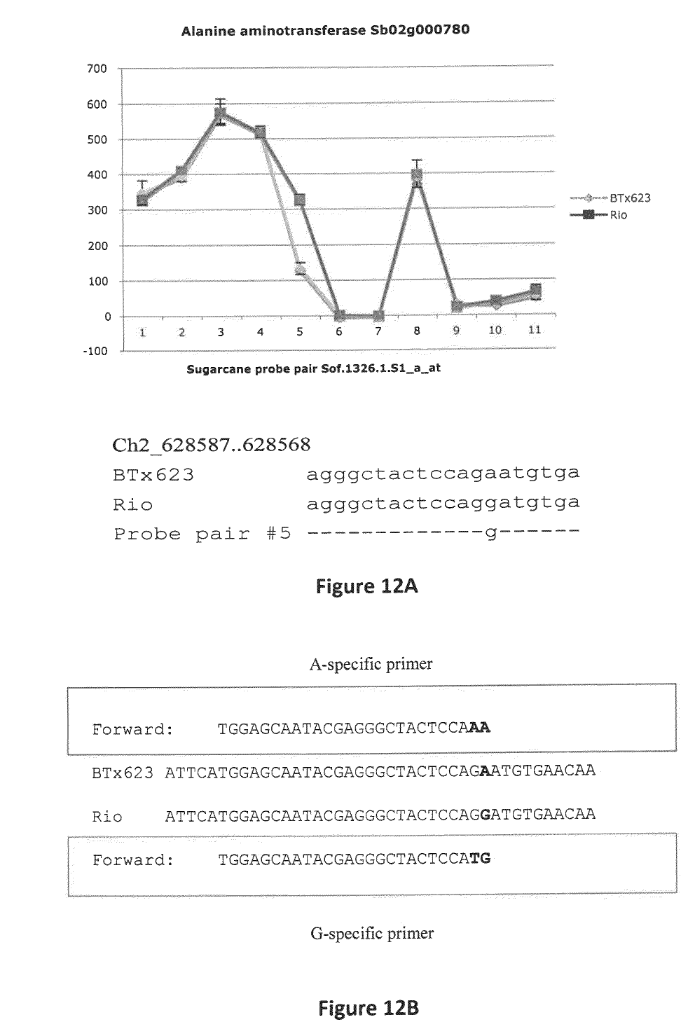

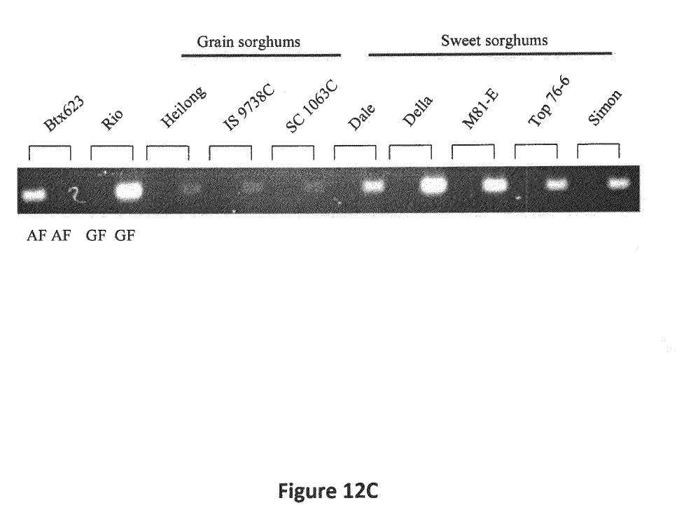

The identification of SNPs between BTx623 and Rio provided a direct way to develop molecular markers that can be used in mapping populations. From 58 candidate genes, we were able to develop allele-specific PCR markers for 18 (Table 7). We utilized the SNAP technique to develop markers based on SNPs (Drenkard et al. 2000), as it is shown for the gene alanine aminotransferase (FIG. 12). These markers were tested also in other grain and sweet sorghum lines to see whether the SNPs were conserved or not (Table 7). In fact, we found a marker within the gene Sb09g029170 that distinguished the grain sorghums from the sweet sorghums cultivars used in this study. The protein product encoded by this gene is a putative ketol-acid reductoisomerase enzyme that is involved in the biosynthesis of valine, leucine and isoleucine amino acids (www.phytozome.net/cgi-bin/gbrowse/sorghum/). SNAP markers were also developed for the cellulose synthase 1 and dolichyl-diphospho-oligosaccharide genes (FIG. 10D).

It has been suggested that Dale and Della sweet sorghums share a common genetic background (Ritter et al. 2007). In agreement with this, we found that from 10 SNAP markers that gave a PCR product in both lines, they always represented the same allele (Table 7). In addition, the sweet sorghum lines Top 76-6 and Simon have been identified as attractive contrasting pairs for mapping purposes based on their difference not only in genetic distance (D) but also in sugar content (measured as Brix degree) (Ali et al. 2008). In our work we identified 6 SNAP markers within the genes Sb01g044810, Sb03g027710, Sb04g0037170, Sb08g008320, Sb09g006050 and Sb10g002230 respectively, which were polymorphic between Top 76-6 and Simon. These markers will be useful for mapping purposes when these lines are used as parents.

Discussion

A significant proportion of the phenotypic variation in any organism can be attributed to polymorphisms at the DNA level. Thus, these DNA polymorphisms can be used for genotyping, molecular mapping, and marker-assisted selection applications. The association of a particular trait of interest with a DNA polymorphism is essential for breeding purposes. Microarrays have been used to identify abundant DNA polymorphisms throughout the genome (Gupta et al. 2008; Hazen and Kay 2003). In particular, ELPs and SFPs can be identified from RNA hybridization studies. SFPs are detected by oligonucleotide arrays and represent DNA polymorphisms between genotypes within an individual oligonucleotide probe pair that is detected by the difference in hybridization affinity (Borevitz et al. 2003). In addition, SFPs present in a transcribed gene may be the underlying cause of the difference in a phenotype of interest. In most of the cases, SNPs are the cause of SFPs as have been demonstrated by sequence analysis (Borevitz et al. 2003; Rostoks et al. 2005).

Here, the goal was to identify SFPs from an Affymetrix sugarcane genechip dataset of closely related species (Calviño et al. 2008). The Affymetrix sugarcane genechip was used to survey the SFPs with the GeSNP software between two sorghum cultivars that differ in the accumulation of fermentable sugars in their stems, with the objective to develop genetic markers for mapping purposes. This is the first report to our knowledge of the use of GeSNP to identify SFPs within closely related grass species and the development of molecular markers based on validated SFPs.

We cloned and sequenced gene fragments harboring SFPs with t-values equal or higher than seven from 58 sweet sorghum genes comprising 125 SFPs in total. In this study, we found a SFP discovery rate (SDR) of 25.6% which is sufficient for most applications. Still, there are several possibilities to increase the SDR. First, the number of biological replicates suggested for using the GeSNP software is 4 or more. In contrast, we had only three replicates for both, grain and sweet sorghum. Second, the cross species hybridization of sorghum RNAs to probe sets of the sugarcane array is not as sensitive as intra species hybridization. Third, false positives could be due to the cross-hybridization of paralogous gene targets to individual probes, which may affect the specificity of the SFP calling. This problem would also arise from using next generation sequencing for SNP detection. Nevertheless, we could show that the use of expression analysis in conjunction with GeSNP is an efficient and inexpensive way to develop new molecular markers.

The sugarcane probe pairs with t-values between 22 and 25 had the highest SDR (80%) found in our study. One of these probe pair sets matched a sorghum gene coding for fructose bisphosphate aldolase (cytoplasmic isozyme) and the identified SFP was confirmed through DNA sequence analysis (FIG. 13). This gene codes for a glycolytic enzyme that catalyzes the cleavage of fructose 1,6 bisphosphate to glyceraldehyde 3-phosphate and dihydroxyacetone phosphate (Tsutsumi et al. 1994).

One third (33%) of the 58 genes that we have sequenced have a validated SFP. In addition, we could detect SNPs in 19% of all sequenced genes at a different position than indicated by GeSNP. This is attributable to the fact that the probe pair set does only cover a part of the gene implies that any SNP outside this region is not reported by GeSNP.

We estimated the average SNP density between BTx623 and Rio to one SNP every 248 bp. This is probably an underestimation because the sugarcane probe sets were designed from genic regions and are, therefore, more conserved than other regions in the genome.

Although the sorghum chromosomes 1, 2, and 3 had the highest numbers for both ELPs and SFPs, chromosomes 8 and 9 were the most polymorphic ones, measured as the number of SNPs per Kb sequence (FIGS. 8 and 11). Our data is in agreement with a previous report by Ritter et al. 2007 in which AFLP markers on chromosome 8 could unambiguously distinguish grain from sweet sorghum lines (Ritter et al. 2007). Furthermore, sugar content QTLs have been located in this chromosome with a RIL derived from a dwarf derivative of Rio as one of the parents.

In addition, we found that a marker within the gene Sb09g029170 coding for a putative ketol-acid reductoisomerase could discriminate the grain sorghums from the sweet sorghum lines used in this study (Table 7). This enzyme is the second in the biosynthesis of branched amino acids valine, leucine and isoleucine (Leung and Guddat 2009).

When the SNPs found through validated SFPs were compared between BTx623, Rio, and sugarcane, we found that SNPs between BTx623 and sugarcane are twice as high as between Rio and sugarcane.

Allelic genetic diversity among sweet sorghum cultivars has previously been investigated based on SSR markers (Ali et al. 2008). This study described the correlations between allelic diversity and the degree of stem sugar. Indeed, one could envision a simpler approach using the microarray described here by hybridizing stem-derived RNAs from these lines to the sugarcane genechip and identify both ELPs and SFPs for subsequent mapping of sugar content QTLs. Furthermore, the SNPs identified in our study provided us with the opportunity to develop molecular markers within genes. So far, there is no report of SNP based molecular markers in transcribed genes in sorghum. The SFPs generated from transcriptome studies are also useful for the development of markers in those species that lack sequence resources such as Miscanthus and switchgrass, further extending the use of microarrays of one species for related ones.

Materials and Methods

Plant Material

The grain sorghum lines Heilong (accession number PI 563518), IS 9738C (PI 595715) and SC 1063C (PI 595741) were obtained from the National Plant Germplasm System (NPGS), USDA. The other lines used in this study were previously described (Calvin et al. 2008). Two weeks old seedlings were harvested for the extraction of genomic DNA.

SFP Discovery and Validation from Affymetrix Transcript Data

The microarray analysis for differentially expressed transcripts in stems of grain and sweet sorghum with a sugarcane genechip was previously described (Calvin et al. 2008). The CEL files from the microarray work were uploaded into the publicly available GeSNP software at porifera.ucsd.edu/˜cabney/cgi-bin/geSNP.cgi and an excel file was obtained with all the probe sets in the array harboring an SFP together with their respective t-values. The excel file also contained the average hybridization intensity between the PM and MM probe pairs (Avg. scaled PM-MM) as well as their variance values that were converted to standard deviations. These values were used to generate the graphs displaying differences in hybridization intensity between BTx623 and Rio along the eleven sugarcane probe pairs for a given probe set.

From the transcripts previously described as being differentially expressed between grain sorghum BTx623 and sweet sorghum Rio, we selected those harboring SFPs with t-values≧7 for further validation through sequencing.

In total, we sequenced gene fragments corresponding to 58 different genes.

Total RNA from Rio stem tissue was extracted at the time of flowering from three independent plants. RNA extraction was performed with the RNeasy Plant Mini Kit from QIAGEN. cDNA synthesis was performed for each of the three samples from 1 μg of total RNA with the SuperScript III First-Strand Synthesis kit from Invitrogen. cDNAs from Rio were pooled respectively and used for the amplification of genes with SFPs.

The RT-PCR products were checked by agarose gel electrophoresis in order to verify that a single band amplification product from each gene was present. The PCR products were purified with the QIAquick PCR Purification kit from Qiagen and cloned into the pGEM-T easy vector from Promega. Twelve clones per gene were sequenced in order to identify any sequencing or reverse transcriptase errors. The consensus sequence for each gene was then used to find SNPs between BTx623 and Rio.

Development of Molecular Markers using WebSNAPER Software

Once a SNP was identified between BTx623 and Rio for a particular gene of interest, the sequence harboring the SNP in question was uploaded into the publicly available WebSNAPER software (pga.mgh.harvard.edu/cgi-bin/snap3/websnaper3.cgi). The SNAP procedure has been previously described (Drenkard et al. 2000). Several primer pairs per SNP were tested and the ones that successfully distinguished the SNP in one line or the other were selected. The primer sequences used to distinguish SNPs are provided in Table 7.

Genomic DNA from two weeks old seedlings was extracted with the PrepEase Genomic DNA Isolation kit from USB. Several concentrations of genomic DNA were tested and 50 ng was used for testing the SNAP primer pairs through PCR. The conditions used for PCR reaction were as follow: 94° C. for 2′, then 30×[94° C. 30″, 64° C. 30″, 72° C. 30″] and a final extension at 72° C. for 2′.

TABLE 4
Sorghum genes with SFPs predicted by the GeSNP software

| | | #Validated | | Sequence |
|---|---|---|---|---|
| Gene ID | #SFPs * | SFPs | #SNPs | length |
| Ch1 | | | | |
| Sb01g005770 | 1 | 0 | 0 | 378 |
| Sb01g049890 | 1 | 1 | 2 | 401 |
| Sb01g002050 | 1 | 0 | 0 | 429 |
| Sb01g033060 | 1 | 0 | 0 | 429 |
| Sb01g013710 | 3 | 0 | 2 | 214 |
| Sb01g043060 | 2 | 0 | 4 | 418 |
| Sb01g046550 | 2 | 0 | 0 | 318 |
| Sb01g003700 | 1 | 0 | 0 | 455 |
| Sb01g011740 | 1 | 0 | 0 | 233 |
| Sb01g006220 | 1 | 0 | 0 | 292 |
| Sb01g009520 | 2 | 0 | 0 | 404 |
| Sb01g016110 | 5 | 0 | 0 | 397 |
| Sb01g044810 | 6 | 0 | 5 | 502 |
| Ch2 | | | | |
| Sb02g006330 | 2 | 1 | 2 | 191 |
| Sb02g000780 | 1 | 1 | 2 | 273 |
| Sb02g005440 | 1 | 0 | 0 | 464 |
| Sb02g036870 | 2 | 0 | 0 | 225 |
| Sb02g022510 | 1 | 0 | 0 | 552 |
| Sb02g006420 | 4 | 2 | 5 | 731 |
| Sb02g009980 | 3 | 2 | 2 | 363 |
| Sb02g032470 | 2 | 0 | 1 | 438 |
| Ch3 | | | | |
| Sb03g039090 | 6 | 4 | 2 | 405 |
| Sb03g037370 | 1 | 1 | 2 | 311 |
| Sb03g009900 | 2 | 0 | 0 | 517 |
| Sb03g037360 | 2 | 0 | 0 | 400 |
| Sb03g013840 | 4 | 0 | 0 | 139 |
| Sb03g012420 | 3 | 2 | 1 | 144 |
| Sb03g007840 | 1 | 0 | 2 | 355 |
| Sb03g037870 | 6 | 0 | 0 | 333 |
| Sb03g045390 | 1 | 0 | 0 | 558 |
| Sb03g027710 | 1 | 0 | 1 | 341 |
| Sb03g003190 | 2 | 0 | 0 | 454 |
| Ch4 | | | | |
| Sb04g028300 | 1 | 0 | 0 | 494 |
| Sb04g027910 | 2 | 0 | 0 | 485 |
| Sb04g021610 | 1 | 0 | 0 | 209 |
| Sb04q037170 | 1 | 1 | 2 | 346 |
| Sb04g019020 | 8 | 3 | 6 | 235 |
| Sb04g005210 | 1 | 1 | 1 | 236 |
| Ch5 | | | | |
| Sb05g001680 | 2 | 1 | 3 | 153 |
| Ch6 | | | | |
| Sb06g015180 | 2 | 0 | 3 | 314 |
| Sb06g026710 | 1 | 0 | 0 | 277 |
| Sb06g029500 | 2 | 0 | 0 | 486 |
| Ch7 | | | | |
| Sb07g001320 | 7 | 0 | 0 | 473 |
| Sb07g005930 | 1 | 1 | 2 | 436 |
| Ch8 | | | | |
| Sb08g008320 | 1 | 1 | 7 | 447 |
| Sb08g016302 | 1 | 0 | 3 | 268 |
| Sb08g020760 | 1 | 0 | 3 | 488 |
| Sb08g015010 | 4 | 0 | 0 | 484 |
| Sb08g002250 | 6 | 5 | 4 | 316 |
| Sb08g002660 | 1 | 0 | 0 | 345 |
| Ch9 | | | | |
| Sb09g000820 | 1 | 1 | 2 | 394 |
| Sb09g023620 | 1 | 0 | 0 | 434 |
| Sb09g006050 | 2 | 2 | 3 | 268 |
| Sb09g005280 | 2 | 1 | 1 | 527 |
| Sb09g029170 | 1 | 0 | 10 | 406 |
| Ch10 | | | | |
| Sb10g002230 | 1 | 0 | 2 | 398 |
| Sb10g007380 | 1 | 1 | 2 | 374 |
| Sb10g004540 | 1 | 0 | 0 | 255 |
| Total | 125 | 32 | 87 | 21612 |

* SFPs with t-values ≧ 7

TABLE 5
Sugarcane probe pairs with t-values of 22-25 that identify sorghum transcripts with SFPs but not ELPs

| Sugarcane probe set | Probe pair # | S. bicolor ID | Position | Function |
|---|---|---|---|---|
| t-value = 22 | | | | |
| Sof.4093.2.S1_at | 6 | NGH* | Ch1_8313833 . . . 8313816 | |
| Sof.4567.1.S1_at | 8 | Sb01g044810 | Ch1_67980922 . . . 67980946 | MADS-box |
| | | | | transcription factor |
| Sof.5184.2.S1_a_at | 6 | Sb03g001160 | Ch3_991187 . . . 991163 | Similar to |
| | | | | Os02g0294700 protein |
| SofAffx.1284.1.S1_s_at | 3 | Sb03g008870 | Ch3_9656668 . . . 9656644 | Unknown |
| Sof.5348.1.S1_at | 11 | Sb03g003510 | Ch3_3731533 . . . 3731509 | Ubiquitin-conjugating |
| | | | | enzyme E2 |
| Sof.2770.1.S1_at | 4 | Sb03g041770 | Ch3_69253777 . . . 69253759 | Unknown |
| Sof.3851.1.S1_at | 10 | Sb05g004130 | Ch5_4878250 . . . 4878268 | 60S ribosomal protein |
| | | | | L3 |
| SofAffx.630.1.S1_at | 5 | Sb05g022890 | Ch5_55221453 . . . 55221432 | GDP-mannose 3,5- |
| | | | | epimerase 1 |
| Sof.2692.1.S1_at | 5 | Sb08g002250 | Ch8_2360780 . . . 2360756 | Cytochrome P450 |
| Sof.4985.2.S1_a_at | 10 | Sb08g018480 | Ch8_48581627 . . . 48581646 | ATP-citrate synthase |
| SofAffx.1129.1.S1_at | 2 | Sb08g021850 | Ch8_53598165 . . . 53598144 | Serine/threonine |
| | | | | protein phosphatase |
| SofAffx.1129.1.S1_at | 9 | Sb08g021850 | Ch8_53598029 . . . 53598005 | Serine/threonine |
| | | | | protein phosphatase |
| Sof.4246.1.S1_a_at | 11 | Sb09g005270 | Ch9_6772194 . . . 6772216 | Unknown |
| t-value = 23 | | | | |
| Sof.2535.1.A1_at | 6 | Sb02g011130 | Ch2_18051363 . . . 18051363 | Similar to putative RES |
| | | | | protein |
| Sof.1519.2.S1_at | 8 | Sb02g006330 | Ch2_7909203 . . . 7909180 | Dolichyl-di- |
| | | | | phosphooligosacharide- |
| | | | | protein |
| Sof.1282.2.S1_a_at | 11 | NGH | Ch2_57946767 . . . 57946743 | |
| Sof.1664.2.S1_a_at | 1 | Sb03g033760 | Ch3_62018464 . . . 62018488 | Putative BURP domain- |
| | | | | containing protein |
| SofAffx.1284.1.S1_x_at | 2 | Sb03g008870 | Ch3_9656190 . . . 9656166 | Unknown |
| Sof.497.2.S1_at | 7 | Sb07g027480 | Ch7_62509159 . . . 62509135 | 3-hydroxy-3- |
| | | | | methylglutaryl-coA |
| | | | | reductase |
| Sof.1190.1.S1_at | 8 | Sb07g005930 | Ch7_8393958 . . . 8393934 | Unknown |
| Sof.2692.1.S1_at | 6 | Sb08g002250 | Ch8_2360760 . . . 2360736 | Cytochrome P450 |
| Sof.355.1.S1_at | 8 | Sb09g005570 | Ch9_7345144 . . . 7345120 | Heat shock protein |
| t-value = 24 | | | | |
| Sof.4310.1.S1_at | 3 | Sb01g028500 | Ch1_49703504 . . . 49703480 | Senescence-associated |
| | | | | protein like |
| Sof.4030.1.A1_at | 10 | Sb02g003450 | Ch2_3915697 . . . 3915680 | Similar to B0616E02- |
| | | | | H0507E05.5 protein |
| Sof.4972.1.S1_a_at | 9 | NGH | Ch3_17046891 . . . 17046867 | |
| Sof.1835.1.S1_at | 3 | Sb03g033140 | Ch3_61527980 . . . 61527956 | Putative nuclear RNA |
| | | | | binding protein A |
| Sof.1003.1.S1_at | 2 | Sb05g002580 | Ch5_2717665 . . . 2717641 | Cytochrome P450 |
| Sof.1694.1.A1_at | 9 | Sb06q033460 | Ch6_61437575 . . . 61437596 | Similar to H0913C04.1 |
| | | | | protein |
| Sof.3020.2.A1_at | 4 | Sb09g002960 | Ch9_3216665 . . . 3216682 | Aspartic proteinase |
| t-value = 25 | | | | |
| Sof.2803.1.S1_at | 11 | Sb01g043050 | Ch1_66375993 . . . 66375971 | Unknown |
| Sof.1537.1.S1_at | 7 | Sb03g011270 | Ch3_12484656 . . . 12484632 | Mg-protoporphyrin IX |
| | | | | monomethyl ester |
| | | | | cyclase |
| Sof.2992.1.A1_at | 6 | Sb04g037920 | Ch4_67480989 . . . 67481008 | Similar to |
| | | | | Os04g0137500 |
| Sof.1443.1.S1_at | 7 | Sb04g010990 | Ch4_15758311 . . . 15758334 | Unknown |
| Sof.3814.1.S1_at | 11 | Sb04g019020 | Ch4_44439307 . . . 44439289 | Fructose-bisphosphate |
| | | | | aldolase |
| Sof.3699.1.A1_at | 4 | Sb07q005850 | Ch7_8311400 . . . 8311376 | Equilibrative nucleoside |
| | | | | transporter 1 |
| Sof.2286.1.A1_at | 2 | Sb09g025350 | Ch9_54815478 . . . 54815502 | Similar to |
| | | | | Os05q051300 |
| Sof.1994.1.S1_x_at | 7 | Sb10g005375 | Ch10_4802664 . . . 4802640 | |

*NGH: Non Genie Hit

TABLE 6
Nucleotide change conservation for validated SFPs between BTx623, Rio and sugarcane

| S. bicolor gene | Position | Sugarcane probe set | Probe pair # | t-value | BTx623-Rio-Sc* SNP |
|---|---|---|---|---|---|
| Sb02g006330 | Ch2_7909203 . . . 7909180 | Sof.1519.2.S1_at | 8 | 23 | C-T-C |
| Sb02g000780 | Ch2_628587 . . . 628568 | Sof.1326.1.S1_a_at | 5 | 15.2 | A-G-G |
| Sb02g006420 | Ch2_804875 . . . 8048728 | Sof.2471.1.S1_at | 5 | 34.1 | C-A-C |
| | Ch2_8048741 . . . 8048717 | | 6 | 19.8 | same |
| Sb02g009980 | Ch2_14533601 . . . 14533625 | SofAffx.868.1.S1_s_at | 9 | 13.7 | A-T-A/ |
| | | | | | C-T-C |
| | Ch2_14533610 . . . 14533630 | | 10 | 12.9 | same** |
| Sb03g037370 | Ch3_65336537 . . . 65336560 | SofAffx.772.1.S1_s_at | 7 | 19.1 | C-G-C |
| Sb03g012420 | Ch3_14371043 . . . 14371019 | Sof.2629.3.S1_a_at | 8 | 38.2 | C-T-C |
| | Ch3_14371036 . . . 14371016 | | 9 | 19.4 | same |
| Sb03g039090 | Ch3_66876720 . . . 66876744 | Sof.5269.1.S1_at | 6 | 8.1 | T-A-T/ |
| | | | | | C-A-C |
| | Ch3_66876724 . . . 66876748 | | 7 | 12 | same |
| | Ch3_66876727 . . . 66876751 | | 8 | 17.1 | same |
| | Ch3_66876730 . . . 66876754 | | 9 | 16.1 | same |
| | Ch3_66876734 . . . 66876758 | | 10 | 45.8 | same |
| Sb04g019020 | Ch4_44439369 . . . 44439345 | Sof.3814.1.S1_at | 8 | 21.9 | C-T-T |
| | Ch4_44439366 . . . 44439342 | | 9 | 15.3 | same |
| | Ch4_44439307 . . . 44439289 | | 11 | 25.5 | T-G-T |
| Sb04g037170 | Ch4_66851287 . . . 66851311 | Sof.151.1.S1_at | 8 | 19.4 | G-C-G |
| Sb05g001680 | Ch5_1816812 . . . 1816788 | Sof.1902.1.S1_s_at | 6 | 33.1 | A-G-G |
| Sb07g005930 | Ch7_8393958 . . . 8393934 | Sof.1190.1.S1_at | 8 | 23.3 | T-G-T |
| Sb08g008320 | Ch8_15917006 . . . 15917030 | SofAffx.1412.1.A1_s_at | 2 | 15.1 | T-C-C |
| Sb08g002250 | Ch8_2360967 . . . 2360943 | Sof.2692.1.S1_at | 2 | 16.8 | A-G-A |
| | Ch8_2360780 . . . 2360756 | | 5 | 22.1 | A-G-G |
| | Ch8_2360760 . . . 2360736 | | 6 | 23.6 | T-C-C |
| Sb09g006050 | Ch9_8732113 . . . 8732094 | SofAffx.1438.1.A1_s_at | 3 | 14.9 | C-G-C |
| | Ch9_8732054 . . . 8732030 | | 7 | 82.5 | C-A-C |
| Sb09g000820 | Ch9_624173 . . . 624197 | Sof.808.1.S1_at | 8 | 29 | G-C-G |
| Sb09g005280 | | | | | |
| Sb10g007380 | Ch10_7220153 . . . 7220177 | SofAffx.287.1.S1_at | 7 | 14 | T-C-C |

*Sc: Sugarcane
**same means that a different probe pair recognize the same SNP

TABLE 7
Primer sequences of SNAP markers within sorghum genes. Sequences are SEQ ID NOs:
29-100, from top to bottom.

| | | | PCR | |
|---|---|---|---|---|
| | | | product | |
| S. bicolor | | | size | Allele |
| gene ID | Allele | WebSNAPER primer sequence | bps | presence * |
| Sb01g043060 | T | F: GTAATATACTGACGCCAAAAGAGGCGGATT | 306 | BT |
| | | R: TCAACTGCTGTTGTCGAGGACATTGG | | |
| | A | F: TGTAATATACTGACGCCAAAAGAGGCGACTT | 307 | Ri-Top |
| | | R: TCAACTGCTGTTGTCGAGGACATTGG | | |
| Sb01g044810 | C | F: CAATCCTGCTCCCCAATCCAGACC | 334 | BT-Da-De-Sim |
| | | R: GATTACGAGATCAGCGGTCTGGAAAGAAA | | |
| | T | F: GCAATCCTGCTCCCCAATCCAGACT | 335 | Ri-He-IS-SC-M81 |
| | | R: GATTACGAGATCAGCGGTCTGGAAAGAAA | | Top |
| Sb02g000780 | A | F: TGGAGCAATACGAGGGCTACTCCAAA | 118 | BT |
| | | R: AATCTTCAGAAACGCTCCATTTGTGCTG | | |
| | G | F: TGGAGCAATACGAGGGCTACTCCATG | 118 | Ri-He-IS-SC-Da-De |
| | | R: AATCTTCAGAAACGCTCCATTTGTGCTG | | M81-Top-Sim |
| Sb02g006330 | G | F: TGTGGTACAGGTACACAAGCGAGAACATG | 115 | BT-IS-Da-De-M81 |
| | | R: CCTTACAGGCATAACGAGTATGAGAGATTCATAACA | | |
| | A | F: CTTATTTGTGGTACAGGTACACAAGCGAGAATAAA | 121 | Ri-Top-Sim |
| | | R: CCTTACAGGCATAACGAGTATGAGAGATTCATAACA | | |
| Sb03g012420 | C | F: GAAGCATTCTTTCCGATACAATATGGCCTATC | 164 | BT-He-SC-M81-Top |
| | | R: TTCGATTAAAGGATTGTTGATGAAACTAGGGG | | Sim |
| | T | F: GAAGCATTCTTTCCGATACAATATGGCCTACT | 164 | Ri-IS-Da |
| | | R: TTCGATTAAAGGATTGTTGATGAAACTAGGGG | | |
| Sb03g007840 | C | F: CCATAAATGTCATTGTGGAGACATCCGTTC | 161 | BT-He-IS-SC-M81 |
| | | R: TGGAACGTCAAAACATTGACCGGAA | | Top |
| | T | F: AAATGTCATTGTGGAGACATCCGGGT | 157 | Ri-Da-Sim |
| | | R: TGGAACGTCAAAACATTGACCGGAA | | |
| Sb03g027710 | T | F: GGTCATCGGTGATGGTGGAGAACCT | 343 | BT |
| | | R: GGGAATTCGATTATGTCCATCACACCC | | |
| | G | F: AGGTCATCGGTGATGGTGGAGATCTG | 344 | Ri-Da-Sim |
| | | R: GGGAATTCGATTATGTCCATCACACCC | | |
| Sb03g039090 | C | F: CGAACCCAACAACCTGTAACAATAAGCACTAC | 326 | BT-Da-De-Top-Sim |
| | | R: GGAATTCGATTATCTCGGGGCTCATCTAC | | |
| | A | F: GAACCCAACAACCTGTAACAATAAGCAGAAA | 325 | Ri-M81 |
| | | R: GGAATTCGATTATCTCGGGGCTCATCTAC | | |
| Sb04g0037170 | G | F: CACAAGCGACTTGAAACTGCGCTG | 131 | BT-IS-SC-Top |
| | | R: GGCTTGACAACTGCTTCAACCTCTGC | | |
| | C | F: CACAAGCGACTTGAAACTGCACCC | 131 | Ri-He-Da-De-M81 |
| | | R: GGCTTGACAACTGCTTCAACCTCTGC | | Sim |
| Sb07g005930 | T | F: CAGTTCTCCAATCCTTTCCTCTGTGGTCT | 146 | BT-He-SC-Da-M81 |
| | | R: GTGAGAAGCGTGGGATGCTCATCAG | | |
| | G | F: GTTCTCCAATCCTTTCCTCTGTGGTCG | 144 | Ri-IS-Top-Sim |
| | | R: GTGAGAAGCGTGGGATGCTCATCAG | | |
| Sb08g020760 | C | F: CAGAGGAAGCCCTTACACAGATCCGAC | 1400 | BT-M81 |
| | | R: TACCCACAGGTCTGGAAAGGGCAAG | | |
| | T | F: CAGAGGAAGCCCTTACACAGATCCGAT | 416 | Ri-He-IS-SC-Top |
| | | R: TACCCACAGGTCTGGAAAGGGCAAG | | Sim |
| Sb089008320 | T | F: GCAGTGGAAGGACATCATTGCCCAT | 174 | BT-He-Da-M81-Sim |
| | | R: CTCTTCCGGGACGCGACGTTC | | |
| | C | F: CAGTGGAAGGACATCATTGCCGTC | 173 | Ri-IS-SC-Top |
| | | R: CTCTTCCGGGACGCGACGTTC | | |
| Sb09g005280 | A | F: GCAGCACCGTCACCGGCACTA | 142 | BT |
| | | R: GAGGCTCAATCAAGATCGTCTGCCC | | |
| | G | F: CAGCACCGTCACCGGCATCG | 141 | Ri-He-IS-SC-Da-De |
| | | R: GAGGCTCAATCAAGATCGTCTGCCC | | M81-Top-Sim |
| Sb09g029170 | C | F: CTACTCTGAGATCATCAACGAGAGCGTGAAC | 124 | BT-He-SC-IS |
| | | R: CCTAGATCCCAGGCGAGCCGTC | | |
| | T | F: CTACTCTGAGATCATCAACGAGAGCGTGTTT | 124 | RI-Da-De-M81-Top |
| | | R: CCTAGATCCCAGGCGAGCCGTC | | Sim |
| Sb09g000820 | G | F: TCGAGAGCGATGCCTTCTGACATTG | | |
| | | R: CCATATCTCCAGCCATCTTCAATGTTGTG | 128 | BT-Top |
| | A | F: CGAGAGCGATGCCTTCTGACAGCA | 130 | Ri |
| | | R: CCATATCTCCAGCCATCTTCAATGTTGTG | | |
| Sb09g006050 | C | F: ATAGAAGGCAGAATGAACGCTGGAAAGC | 105 | BT-Top |
| | | R: GGGCAAGCAGGCCTGGAACTTC | | |
| | A | F: AGAAGGCAGAATGAACGCTGGACTGA | 103 | Ri-He-IS-SC-Da-De |
| | | R: GGGCAAGCAGGCCTGGAACTTC | | M81-Sim |
| Sb10g007380 | T | F: GAACTACAGACATGCACAAGGATAGCAGGTT | 561 | BT-Top |
| | | R: ATTGCATTCAGGAAGCTCGCTCGA | | |
| | C | F: GAACTACAGACATGCACAAGGATAGCAGAGC | 561 | Ri-He-IS-SC-Da-De |
| | | R: ATTGCATTCAGGAAGCTCGCTCGA | | M81 |
| Sb10g002230 | G | F: CTTCAATCCGACAACCAAGTCGCTG | 197 | BT-He-IS-Top |
| | | R: CTGGAACTGCAATGCGGCCATT | | |
| | A | F: GCTTCAATCCGACAACCAAGTCGCTA | 197 | Ri-SC-Da-De-M81 |
| | | R: CTGGAACTGCAATGCGGCCATT | | Sim |

BT × 623 (BT); Rio (Ri); Heilong (He); IS 9738C (IS); SC 1063C (SC); Dale (Da); Della (De); M81-E (M81); Top76-6 (Top);
Simon (Sim)
Only the cultivars that gave a PCR product were scored. If a cultivar was heterozygous for a particular
allele was not scored it.

### REFERENCES

Bateman, A., Bycroft, M. (2000). The structure of a LysM domain from E. coli membrane-bound lytic murein transglycosylase D (MltD). J Mol Biol 299, 1113-1119.

Bateman, A., and Bycroft, M. (2000). The structure of a LysM domain from E. coli membrane-bound lytic murein transglycosylase D (MltD). J Mol Biol 299, 1113-1119.

Bennetzen, J. L., and Freeling, M. (1993). Grasses as a single genetic system: genome composition, collinearity and compatibility. Trends Genet 9, 259-261.

Bull, T., and Glasziou, K. (1963). The evolutionary significance of sugar accumulation in Saccarhum. Aust J Biol Sci 16, 737-742.

Burk, D. H., and Ye, Z. H. (2002). Alteration of oriented deposition of cellulose microfibrils by mutation of a katanin-like microtubule-severing protein. Plant Cell 14, 2145-2160.

Casu, R. E., Jarmey, J. M., Bonnett, G. D., and Manners, J. M. (2007). Identification of transcripts associated with cell wall metabolism and development in the stem of sugarcane by Affymetrix GeneChip Sugarcane Genome Array expression profiling. Funct Integr Genomics 7, 153-167.

Casu, R. E., Grof, C. P., Rae, A. L., McIntyre, C. L., Dimmock, C. M., and Manners, J. M. (2003). Identification of a novel sugar transporter homologue strongly expressed in maturing stem vascular tissues of sugarcane by expressed sequence tag and microarray analysis. Plant Mol Biol 52, 371-386.

Chapple, C., and Carpita, N. (1998). Plant cell walls as targets for biotechnology. Curr Opin Plant Biol 1, 179-185.

D'Hont, A., Grivet, L., Feldmann, P., Rao, S., Berding, N., and Glaszmann, J. C. (1996). Characterization of the double genome structure of modern sugarcane cultivars (Saccharum spp.) by molecular cytogenetics. Mol Gen Genet 250, 405-413.

Faik, A., Abouzouhair, J., and Sarhan, F. (2006). Putative fasciclin-like arabinogalactan-proteins (FLA) in wheat (Triticum aestivum) and rice (Oryza sativa): identification and bioinformatic analyses. In Mol Genet Genomics, pp. 478-494.

Gale, M. D., and Devos, K. M. (1998). Comparative genetics in the grasses. Proc Natl Acad Sci USA 95, 1971-1974.

Gremme, G., Brendel, V., Sparks, M. E., and Kurtz, S. (2005). Engineering a software tool for gene structure prediction in higher organisms. Information and Software Technology 47, 965.

Grivet, L., and Arruda, P. (2002). Sugarcane genomics: depicting the complex genome of an important tropical crop. Curr Opin Plant Biol 5, 122-127.

Henrissat, B., Callebaut, I., Fabrega, S., Lehn, P., Mornon, J. P., and Davies, G. (1996). Conserved catalytic machinery and the prediction of a common fold for several families of glycosyl hydrolases. Proc Natl Acad Sci USA 93, 5674.

Hoffman-Thoma, G., Hinkel, K., Nicolay, P., and Willenbrink, J. (1996). Sucrose accumulation in sweet sorghum stem internodes in relation to growth. Physiologia Plantarum 97, 277-284.

International Rice Genome Sequencing, P. (2005). The map-based sequence of the rice genome. Nature 436, 793-800.

Ishimaru, K., Hirotsu, N., Madoka, Y., and Kashiwagi, T. (2007). Quantitative trait loci for sucrose, starch, and hexose accumulation before heading in rice. Plant Physiol Biochem 45, 799-804.

Jong, J. C., León, P., Zhou, L., and Sheen, J. (1997). Hexokinase as a sugar sensor in higher plants. In The Plant Cell.

Juge, N., Nohr, J., Le Gal-Coeffet, M. F., Kramhoft, B., Furniss, C. S., Planchot, V., Archer, D. B., Williamson, G., and Svensson, B. (2006). The activity of barley alpha-amylase on starch granules is enhanced by fusion of a starch binding domain from Aspergillus niger glucoamylase. Biochim Biophys Acta 1764, 275-284.

Kawamoto, T., Noshiro, M., Shen, M., Nakamasu, K., Hashimoto, K., Kawashima-Ohya, Y., Gotoh, O., and Kato, Y. (1998). Structural and phylogenetic analyses of RGD-CAP/beta ig-h3, a fasciclin-like adhesion protein expressed in chick chondrocytes. Biochim Biophys Acta 1395, 288-292.

Kellogg, E. A. (2001). Evolutionary history of the grasses. Plant Physiol 125, 1198-1205.

Koch, K. (2004). Sucrose metabolism: regulatory mechanisms and pivotal roles in sugar sensing and plant development. Curr Opin Plant Biol 7, 235-246.

Lingle, S. (1987). Sucrose metabolism in the primary culm of sweet sorghum during development. Crop Science 27, 1214-1219.

Lukowitz, W., Nickle, T. C., Meinke, D. W., Last, R. L., Conklin, P. L., and Somerville, C. R. (2001). Arabidopsis cyt1 mutants are deficient in a mannose-1-phosphate guanylyltransferase and point to a requirement of N-linked glycosylation for cellulose biosynthesis. Proc Natl Acad Sci USA 98, 2262-2267.

McCormick, A. J., Cramer, M. D., and Watt, D. A. (2006). Sink strength regulates photosynthesis in sugarcane. New Phytol 171, 759-770.

Messing, J., and Llaca, V. (1998). Importance of anchor genomes for any plant genome project. Proceedings of the National Academy of Sciences of the United States of America 95, 2017-2020.

Messing, J., and Bennetzen, J. (2008). Grass genome structure and evolution. Genome Dynamics 4, 41-56.

Ming, R., Liu, S. C., Moore, P. H., Irvine, J. E., and Paterson, A. H. (2001). QTL analysis in a complex autopolyploid: genetic control of sugar content in sugarcane. Genome Res 11, 2075-2084.

Munford, R. S., Sheppard, P. O., and O'Hara, P. J. (1995). Saposin-like proteins (SAPLIP) carry out diverse functions on a common backbone structure. In The Journal of Lipid Research.

Passardi, F., Penel, C., and Dunand, C. (2004). Performing the paradoxical: how plant peroxidases modify the cell wall. Trends Plant Sci 9, 534-540.

Pego, J. V., and Smeekens, S. C. (2000). Plant fructokinases: a sweet family get-together. Trends Plant Sci 5, 531-536.

Ragauskas, A. J., Williams, C. K., Davison, B. H., Britovsek, G., Cairney, J., Eckert, C. A., Frederick, W. J., Jr., Hallett, J. P., Leak, D. J., Liotta, C. L., Mielenz, J. R., Murphy, R., Templer, R., and Tschaplinski, T. (2006). The path forward for biofuels and biomaterials. Science 311, 484-489.

Rohwer, J. M., and Botha, F. C. (2001). Analysis of sucrose accumulation in the sugar cane culm on the basis of in vitro kinetic data. Biochem J 358, 437-445.

Schreiber, V., Dantzer, F., Ame, J. C., and de Murcia, G. (2006). Poly(ADP-ribose): novel functions for an old molecule. Nat Rev Mol Cell Biol 7, 517-528.

Somerville, C., Bauer, S., Brininstool, G., Facette, M., Hamann, T., Milne, J., Osborne, E., Paredez, A., Persson, S., Raab, T., Vorwerk, S., and Youngs, H. (2004). Toward a systems approach to understanding plant cell walls. Science 306, 2206-2211.

Song, R., Segal, G., and Messing, J. (2004). Expression of the sorghum 10-member kafirin gene cluster in maize endosperm. Nucleic acids research 32, e189.

Stokeley, D., Bemporad, D., Gavaghan, D., and Sansom, M. S. (2007). Conformational Dynamics of a Lipid-Interacting Protein: MD Simulations of Saposin B. In Biochemistry, pp. 13573-13580.

Tarpley, L., Lingle, S., Vietor, D. M., Andrews, D., and Miller, F. (1994). Enzymatic control of nonstructural carbohydrate concentrations in stems and panicles of sorghum. Crop Science 34, 446-452.

Uys, L., Botha, F. C., Hofmeyr, J. H., and Rohwer, J. M. (2007). Kinetic model of sucrose accumulation in maturing sugarcane culm tissue. Phytochemistry 68, 2375-2392.

Vanderauwera, S., De Block, M., Van de Steene, N., van de Cotte, B., Metzlaff, M., and Van Breusegem, F. (2007). Silencing of poly(ADP-ribose) polymerase in plants alters abiotic stress signal transduction. Proc Natl Acad Sci USA 104, 15150-15155.

Wolucka, B. A., and Van Montagu, M. (2003). GDP-mannose 3′,5′-epimerase forms GDP-L-gulose, a putative intermediate for the de novo biosynthesis of vitamin C in plants. J Biol Chem 278, 47483-47490.

Xue, G. P., McIntyre, C. L., Jenkins, C. L., Glassop, D., van Herwaarden, A. F., and Shorter, R. (2008). Molecular dissection of variation in carbohydrate metabolism related to water-soluble carbohydrate accumulation in stems of wheat. Plant Physiol 146, 441-454.

Yang, J., and Zhang, J. (2006). Grain filling of cereals under soil drying. New Phytol 169, 223-236.

Yang, J., Sardar, H. S., McGovern, K. R., Zhang, Y., and Showalter, A. M. (2007). A lysine-rich arabinogalactan protein in Arabidopsis is essential for plant growth and development, including cell division and expansion. Plant J 49, 629-640.

Zhou, R., Cheng, L., and Dandekar, A. M. (2006). Down-regulation of sorbitol dehydrogenase and up-regulation of sucrose synthase in shoot tips of the transgenic apple trees with decreased sorbitol synthesis. J Exp Bot 57, 3647-3657.

Ali M, Rajewski J, Baenziger P, Gill K, Eskridge K, Dweikat I (2008) Assessment of genetic diversity and relationship among a collection of US sweet sorghum germplasm by SSR markers. Molecular Breeding 21: 497-509

Borevitz J O, Chory J (2004) Genomics tools for QTL analysis and gene discovery. Current Opinion in Plant Biology 7: 132-136

Borevitz J O, Hazen S P, Michael T P, Morris G P, Baxter I R, Hu T T, Chen H, Werner J D, Nordborg M, Salt D E, Kay S A, Chory J, Weigel D, Jones J D, Ecker J R (2007) Genome-wide patterns of single-feature polymorphism in Arabidopsis thaliana. Proc Natl Acad Sci USA 104: 12057-12062

Borevitz J O, Liang D, Plouffe D, Chang H S, Zhu T, Weigel D, Berry C C, Winzeler E, Chory J (2003) Large-scale identification of single-feature polymorphisms in complex genomes. Genome Res 13: 513-523

Cáceres M, Lachuer J, Zapala M A, Redmond J C, Kudo L, Geschwind D H, Lockhart D J, Preuss T M, Barlow C (2003) Elevated gene expression levels distinguish human from non-human primate brains. Proc Natl Acad Sci USA 100: 13030-13035

Calviño M, Bruggmann R, Messing J (2008) Screen of genes linked to high-sugar content in stems by comparative genomics. Rice 1: 166-176

Coram T E, Settles M L, Wang M, Chen X (2008) Surveying expression level polymorphism and single-feature polymorphism in near-isogenic wheat lines differing for the Yr5 stripe rust resistance locus. Theor Appl Genet 117: 401-411

Das S, Bhat P R, Sudhakar C, Ehlers J D, Wanamaker S, Roberts P A, Cui X, Close T J (2008) Detection and validation of single feature polymorphisms in cowpea (Vigna unguiculata L. Walp) using a soybean genome array. BMC Genomics 9: 107

Drenkard E, Richter B G, Rozen S, Stutius M L, Angell N A, Mindrinos M, Cho J R, Oefner P J, Davis R W, Ausubel F M (2000) A simple procedure for the analysis of single nucleotide polymorphisms facilitates map-based cloning in Arabidopsis. Plant Physiology 124: 1483-1492

Greenhall J A, Zapala M A, Cáceres M, Libiger O, Barlow C, Schork N J, Lockhart D J (2007) Detecting genetic variation in microarray expression data. Genome Res 17: 1228-1235

Gupta P K, Rustgi S, Mir R R (2008) Array-based high-throughput DNA markers for crop improvement. Heredity 101: 5-18

Hazen S P, Borevitz J O, Harmon F G, Pruneda-Paz J L, Schultz T F, Yanovsky M J, Liljegren S J, Ecker J R, Kay S A (2005) Rapid array mapping of circadian clock and developmental mutations in Arabidopsis. Plant Physiology 138: 990-997

Hazen S P, Kay S A (2003) Gene arrays are not just for measuring gene expression. Trends in Plant Science 8: 413-416

Jansen R C, Nap J P (2001) Genetical genomics: the added value from segregation. Trends in Genetics 17: 388-391

Kumar R, Qiu J, Joshi T, Valliyodan B, Xu D, Nguyen H T (2007) Single feature polymorphism discovery in rice. PLoS ONE 2: e284

Leung E W, Guddat L W (2009) Conformational Changes in a Plant Ketol-Acid Reductoisomerase upon Mg(2+) and NADPH Binding as Revealed by Two Crystal Structures. J Mol Biol DOI 10.1016/j.jmb.2009.04.012

Paterson A H, Bowers J E, Bruggmann R, Dubchak I, Grimwood J, Gundlach H, Haberer G, Hellsten U, Mitros T, Poliakov A, Schmutz J, Spannagl M, Tang H, Wang X, Wicker T, Bharti A K, Chapman J, Feltus F A, Gowik U, Grigoriev I V, Lyons E, Maher C A, Martis M, Narechania A, Otillar R P, Penning B W, Salamov A A, Wang Y, Zhang L, Carpita N C, Freeling M, Gingle A R, Hash C T, Keller B, Klein P, Kresovich S, McCann M C, Ming R, Peterson D G, Mehboob-ur-Rahman, Ware D, Westhoff P, Mayer K F, Messing J, Rokhsar D S (2009) The Sorghum bicolor genome and the diversification of grasses. Nature 457: 551-556

Potokina E, Druka A, Luo Z, Wise R, Waugh R, Kearsey M (2008) Gene expression quantitative trait locus analysis of 16 000 barley genes reveals a complex pattern of genome-wide transcriptional regulation. Plant J 53: 90-101

Ritter K B, McIntyre C L, Godwin I D, Jordan D R, Chapman S C (2007) An assessment of the genetic relationship between sweet and grain sorghums, within Sorghum bicolor ssp. bicolor (L.) Moench, using AFLP markers. Euphytica 157: 161-176

Rostoks N, Borevitz J O, Hedley P E, Russell J, Mudie S, Morris J, Cardle L, Marshall D F, Waugh R (2005) Single-feature polymorphism discovery in the barley transcriptome. Genome Biol 6: R54

Shiu S H, Borevitz J O (2008) The next generation of microarray research: applications in evolutionary and ecological genomics. Heredity 100: 141-149

Tsutsumi K, Kagaya Y, Hidaka S, Suzuki J, Tokairin Y, Hirai T, Hu D L, Ishikawa K, Ejiri S (1994) Structural analysis of the chloroplastic and cytoplasmic aldolase-encoding genes implicated the occurrence of multiple loci in rice. Gene 141: 215-220

Varshney R K, Graner A, Smells M E (2005) Genomics-assisted breeding for crop improvement. Trends in Plant Science 10: 621-630

Werner J D, Borevitz J O, Warthmann N, Trainer G T (2005) Quantitative trait locus mapping and DNA array hybridization identify an FLM deletion as a cause for natural flowering-time variation. Proc Natl Acad Sci USA 102: 2460-2465

West M A, van Leeuwen H, Kozik A, Kliebenstein D J, Doerge R W, St Clair D A, Michelmore R W (2006) High-density haplotyping with microarray-based expression and single feature polymorphism markers in Arabidopsis. Genome Res 16: 787-795

Xu J H, Messing J (2008) Organization of the prolamin gene family provides insight into the evolution of the maize genome and gene duplications in grass species. Proc Natl Acad Sci USA 105: 14330-14335

Zhu T, Salmeron J (2007) High-definition genome profiling for genetic marker discovery. Trends in Plant Science 12: 1360-1385

1-33. (canceled)

34. A composition comprising at least one miRNA 172 nucleic acid in a biologically compatible carrier, said miRNA modulating flowering time, sugar content and biomass in a sorghum plant via alteration of target gene expression of one or more proteins which regulate sugar metabolism and, or, flowering.

35. The composition of claim 34, wherein said at least one miRNA is miRNA172a or miRNA172c and is cloned into an expression vector.

36. The composition of claim 35, wherein said protein regulates sugar metabolism in the sorghum plant.

37. The composition of claim 35, wherein said protein regulates flowering in the sorghum plant.

38. A method for modulating a biological parameter selected from the group consisting of flowering, sugar metabolism, and biomass production in a sorghum plant or plant cell comprising contacting said plant or plant cell with an effective amount of the composition as claimed in claim 34.

39. The method of claim 38, wherein said miRNA172 is miRNA172a or miRNA172c.

40. The method of claim 38, wherein said miRNA is effective to modulate flowering time in said plant.

42. The method of claim 38, wherein said miRNA is effective to modulate sugar metabolism in said plant.

43. A plant comprising the composition of claim 34 or 35.

44. A biomarker for differentiating sweet sorghum cultivars from grain sorghums comprising assessing said plant for the presence or absence of a marker within the gene Sb09g029170, said marker encoding a ketol-acid reductoisomerase enzyme.
